(19) United States
(12) Patent Application Publication
(10) Pub. No.: US 2025/0382624 A1
(43) Pub. Date: Dec. 18, 2025

(54) METHODS AND COMPOSITIONS TO PROMOTE TARGETED GENOME MODIFICATIONS USING HUH ENDONUCLEASES

(71) Applicant: Monsanto Technology LLC, St. Louis, MO (US)

(72) Inventor: Ervin D. NAGY, Lake Saint Louis, MO (US)

(73) Assignee: Monsanto Technology LLC, St. Louis, MO (US)

(21) Appl. No.: 19/251,205

(22) Filed: Jun. 26, 2025

Related U.S. Application Data
(63) Continuation of application No. 17/632,435, filed on Feb. 2, 2022, now Pat. No. 12,371,695.
(60) Provisional application No. 62/882,266, filed on Aug. 2, 2019.

(51) Int. Cl. C12N 15/52; C12N 15/64; C12Q 1/6811

(52) U.S. Cl. CPC C12N 15/52; C12N 15/64; C12Q 1/6811; C07K 2319/00; C12N 2310/20

(57) ABSTRACT

This disclosure provides compositions and methods for improving site-directed integration of nucleic acids using RNA-guided nucleases coupled with HUH endonucleases.

### CROSS-REFERENCE TO RELATED APPLICATIONS

This application is a continuation of U.S. application Ser. No. 17/632,435, filed internationally on Jul. 31, 2020, which is a U.S. national stage application under 35 U.S.C. § 371 of International Application No. PCT/US2020/044501, filed internationally on Jul. 31, 2020, which claims priority to U.S. Provisional Application No. 62/882,266, filed Aug. 2, 2019, each of which is incorporated herein by reference in their entirety.

### REFERENCE TO AN ELECTRONIC SEQUENCE LISTING

The content of the electronic sequence listing (777052059101subseqlist.xml; Size: 76,871 bytes; and Date of Creation: Jul. 30, 2025) is herein incorporated by reference in its entirety.

### FIELD

The present disclosure relates to compositions and methods related to using RNA-guided nucleases linked to HUH endonucleases to improve targeted integrations of desired sequences into genomes.

### BACKGROUND

CRISPR (Clustered Regularly Interspaced Short Palindromic Repeats) nucleases (e.g., Cas12a, CasX, Cas9) are proteins guided by guide RNAs to a target nucleic acid molecule, where the nuclease can cleave one or two strands of a target nucleic acid molecule. HUH endonucleases are nucleases comprising a HUH (histidine-hydrophobic amino acid-histidine) tag that can form covalent bonds with specific single-stranded DNA sequences.

This disclosure demonstrates that Cas12a nucleases can be tethered to a HUH endonuclease using a novel linker amino acid to improve site-directed integration of a template sequence into a target DNA molecule.

### BRIEF DESCRIPTION OF DRAWINGS

FIG. 1 comprises panels 1A and 1B. FIG. 1A depicts a graphical representation of an RNA-guided nuclease (Cas12a; equivalent to Cas12a) and a histidine- hydrophobic-histidine endonuclease (HUH EN) tethered to a single-stranded DNA (ssDNA) template comprising an HUH recognition sequence (ori) to increase cleavage (scissors) of double-stranded (ds) chromosomal DNA. FIG. 1B depicts a graphical representation of a C-terminal cys-free LbCas12a:HUH fusion expression construct and an N-terminal HUH:cys-free LbCas12a fusion expression construct.

FIG. 2 depicts quantification of targeted indels by next generation sequencing at three target sites. Cys-free LbCas12a:HUH fusions and cys-free LbCas12a had comparable activities. Bars represent average indel rates of four technical replicates. Both experimental (cys-freeLbCas12a:HUH/crRNA/template) and positive controls (cys-free LbCas12a/crRNA/template) produced significantly higher percentages of indels compared to negative controls (crRNA/template) at p=0.05. “t” refers to template.

FIG. 3A shows a pictorial representations of test and control systems for testing fusion proteins in planta. The control system lacks an ori sequence required for the ssDNA template to bind to the HUH endonuclease. FIG. 3B shows results from two independent particle bombardment experiments using the test and control systems shown FIG. 3A. Chromosome cleavage activities of Cas12a were determined by quantifying targeted indels within a seven nucleotide target site. Plants that generated at least 20% targeted indel reads are considered mutants.

FIG. 4 depicts the genomic region flanking the GmTS1 target site and the matching, exogenous template added to the ribonucleoprotein complexes. The differential, middle signature motif of the template is shaded. Upon double-strand chromosome cutting, the template can integrate by non-homologous end-joining (NHEJ) or homologous recombination (HR). After sequencing, the presence/absence of the middle signature motif indicates targeted integration. The nature of integration, e.g., NHEJ, HR or a combination thereof is indicated by the sequences at the 5′ and 3′ chromosome-template junctions as shown. Not shown in this illustration is the ori recognition sequence that was present at the 5′ end of the template used in the test samples. The ori recognition sequence was not present in the negative controls. Black triangles indicate PCR primers.

FIG. 5 shows the results from in planta testing of cys-free LbCas12a:HUH fusion proteins for targeted integrations in RO plants.

FIG. 6 shows the expected editing outcomes when RNP complexes comprising N-terminal or C-Terminal HUH fusion protein, crRNA targeting the GmTS1 genomic site, and templates comprising either a single-stranded DNA template (ss template) or a double-stranded DNA oligo (dsOligo) are concomitantly delivered into soy protoplasts.

FIG. 7 shows chromosome cutting by N-terminal (HUH:cys-free LbCas12a) and C-Terminal (cys-free LbCas12a:HUH) fusion derivatives of LbCas12a across sixteen treatments (Tr) including various combinations of the enzymes, the cognate crRNA, ss templates with or without the ori sequence and a dsOligo with DNA sequence unrelated to the target region. See Table 7 for combination of reagents in each protoplast treatment. Chromosome cutting was quantified by targeted indel rates. Bars represent averages of four biological replicates, error bars are standard deviations. Statistical significance among a few important treatments are illustrated by horizontal lines above the bars.

FIG. 8 shows targeted integrations of templates using N-terminal (HUH:cys-free LbCas12a) and C-terminal (cys-free LbCas12a:HUH) fusion derivatives of LbCas12a. Bars represent averages of four biological replicates, error bars are standard deviations. Total integrations by NHEJ (sum of targeted single-and multiple-copy integrations; black bars), single-copy integrations by NHEJ (dark gray bars) and templated edits by HR (light gray bars) were all significantly better when the templates were tethered to the RNP complexes. See Table 7 for combination of reagents in each treatment. Statistical significance among a few important treatments are illustrated by horizontal lines above the bars.

FIG. 9 shows targeted integrations of an exogenous, untethered ds oligonucleotide (90bp) by NHEJ using N-terminal (HUH:cys-free LbCas12a) and C-terminal (cys-free LbCas12a:HUH) fusion derivatives of LbCas12a. Bars represent averages of four biological replicates, error bars are standard deviations. “dsDNA oligo, Total” indicates sum of targeted single-and multiple-copy integrations by NHEJ (black bars). “dsDNA oligo, 1×” indicates single-copy integrations by NHEJ (dark gray bars). See Table 7 for combination of reagents in each treatment. Statistical significance among a few important treatments are illustrated by horizontal lines above the bars.

### SUMMARY

In one aspect, this disclosure provides a ribonucleoprotein comprising: (a) a recombinant polypeptide comprising: (i) an amino acid sequence encoding a Cas12a nuclease; (ii) an amino acid sequence encoding a linker; and (iii) an amino acid sequence encoding a HUH nuclease; and (b) at least one guide nucleic acid.

In one aspect, this disclosure provides a recombinant nucleic acid comprising: (a) a first nucleic acid sequence encoding a Cas12a nuclease; (b) a second nucleic acid sequence encoding a linker; and (c) a third nucleic acid sequence encoding a HUH nuclease.

In one aspect, this disclosure provides a method of generating an edit in a target DNA molecule comprising contacting the target DNA molecule with a ribonucleoprotein, where the ribonucleoprotein comprises: (a) a recombinant polypeptide comprising: (i) an amino acid sequence encoding a Cas12a nuclease; (ii) an amino acid sequence encoding a linker; and (iii) an amino acid sequence encoding a HUH nuclease; (b) at least one guide nucleic acid; and (c) at least one template nucleic acid molecule, where the ribonucleoprotein generates at least one edit in the target DNA molecule.

In one aspect, this disclosure provides a method of generating an edit in a target DNA molecule comprising providing to a cell: (a) a recombinant polypeptide comprising: (i) an amino acid sequence encoding a Cas 12a nuclease; (ii) an amino acid sequence encoding a linker; and (iii) an amino acid sequence encoding a HUH nuclease, or one or more nucleic acid molecules encoding the recombinant polypeptide; (b) at least one guide nucleic acid, or at least one nucleic acid molecule encoding the at least one guide nucleic acid; and (c) at least one template nucleic acid molecule, or at least one nucleic acid molecule encoding the at least one template nucleic acid molecule, where the recombinant polypeptide, at least one guide nucleic acid, and at least one template nucleic acid molecule form a ribonucleoprotein, and where the ribonucleoprotein generates at least one edit in the target DNA molecule within the cell.

### DETAILED DESCRIPTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N.F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

In an aspect, this disclosure provides a ribonucleoprotein comprising (a) a recombinant polypeptide comprising: (i) an amino acid sequence encoding a Cas12a nuclease; (ii) an amino acid sequence encoding a linker; and (iii) an amino acid sequence encoding a HUH endonuclease; and (b) at least one guide nucleic acid. In an aspect, a ribonucleoprotein further comprises at least one template nucleic acid molecule.

In an aspect, this disclosure provides a ribonucleoprotein comprising (a) a recombinant polypeptide comprising: (i) an amino acid sequence encoding a CasX nuclease; (ii) an amino acid sequence encoding a linker; and (iii) an amino acid sequence encoding a HUH endonuclease; and (b) at least one guide nucleic acid. In an aspect, a ribonucleoprotein further comprises at least one template nucleic acid molecule.

In an aspect, this disclosure provides a recombinant nucleic acid comprising: (a) a first nucleic acid sequence encoding a Cas12a nuclease; (b) a second nucleic acid sequence encoding a linker; and (c) a third nucleic acid sequence encoding a HUH endonuclease. In an aspect, this disclosure provides a recombinant nucleic acid comprising: (a) a first nucleic acid sequence encoding a CasX nuclease; (b) a second nucleic acid sequence encoding a linker; and (c) a third nucleic acid sequence encoding a HUH endonuclease. In an aspect, a recombinant nucleic acid further comprises (d) a fourth nucleic acid sequence encoding at least one guide nucleic acid. In an aspect, a recombinant nucleic acid further comprises (d) a fourth nucleic acid sequence encoding at least one template nucleic acid molecule. In another aspect, a recombinant nucleic acid further comprises (d) a fourth nucleic acid sequence encoding at least one guide nucleic acid; and (e) a fifth nucleic acid sequence encoding at least one template nucleic acid molecule.

CRISPR (Clustered Regularly Interspaced Short Palindromic Repeats) nucleases (e.g., Cas9, CasX, Cas12a (also referred to as Cpf1), CasY) are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome.

CRISPR nucleases associate with their respective crRNAs in their active forms. CasX, similar to the class II endonuclease Cas9, requires another non-coding RNA component, referred to as a trans-activating crRNA (tracrRNA), to have functional activity. Nucleic acid molecules provided herein can combine a crRNA and a tracrRNA into one nucleic acid molecule in what is herein referred to as a “single guide RNA” (sgRNA). Cas12a does not require a tracrRNA to be guided to a target site; a crRNA alone is sufficient for Cas12a. The gRNA guides the active CRISPR nuclease complex to a target site, where the CRISPR nuclease can cleave the target site.

When an RNA-guided CRISPR nuclease and a guide RNA form a complex, the whole system is called a “ribonucleoprotein.” Ribonucleoproteins provided herein can also comprise additional nucleic acids, such as, without being limiting, template nucleic acid molecules. Ribonucleoproteins provided herein can also comprise additional proteins, such as linkers and HUH endonucleases.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cas12a) from the PAM site. PAM sites are only required for type I and type II CRISPR associated proteins, and different CRISPR endonucleases recognize different PAM sites. Without being limiting, Cas12a can recognize at least the following PAM sites: TTTN, and YTN; and CasX can recognize at least the following PAM sites: TTCN, TTCA, and TTC (where T is thymine; C is cytosine; A is adenine; Y is thymine or cytosine; and N is thymine, cytosine, guanine, or adenine).

Cas12a is an RNA-guided nuclease of a class II, type V CRISPR/Cas system. Cas12a nucleases generate staggered cuts when cleaving a target nucleic acid molecule.

In an aspect, a Cas 12a nuclease provided herein is a Lachnospiraceae bacterium Cas12a (LbCas12a) nuclease. In another aspect, a Cas12a nuclease provided herein is a Francisella novicida Cas12a (FnCas12a) nuclease. In some embodiments, the amino acid sequence of the Cas 12a nuclease has been engineered to remove cysteines.

In an aspect, a Cas12a nuclease, or a nucleic acid encoding a Cas12a nuclease, is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Legionella, Francisella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In one embodiment, a Cas12a nuclease provided herein comprises an amino acid sequence at least 80% identical or similar to amino acid sequence disclosed in SEQ ID NO: 1 encoding a cys-free LbCas12a. As used herein, a “cys-free LbCas12a” refers to an LbCas12a protein variant wherein the 9 cysteines present in the native LbCas12a sequence (WO2016205711-1150) are all mutated. In an aspect the cys-free LbCas12a comprises the following 9 amino acid substitutions when compared to a wt LbCas12a protein sequence: C10L, C175L, C565S, C632L, C805A, C912V, C965S, C1090P, C1116L. Cysteine residues in a protein are able to form disulfide bridges providing a strong reversible attachment between cysteines. To control and direct the attachment of Cas12a in a targeted manner the native cysteines are removed to control the formation of these bridges. Not wishing to be bound by a particular theory, removal of the cysteines from the protein backbone would enable targeted insertion of new cysteine residues to control the placement of these reversible connections by a disulfide linkage. This could be between protein domains or to a particle such as a gold particle for biolistic delivery. A tag comprising several residues of cysteine could be added to the cys-free LbCas12a that would allow it to specifically attach to metal beads (specifically gold) in a uniform way.

In an aspect, a Cas12a nuclease provided herein comprises an amino acid sequence at least 80% identical or similar to an amino acid sequence selected from the group consisting of SEQ ID NOs: 1, 15 and 26. In another aspect, a Cas12a nuclease provided herein comprises an amino acid sequence at least 85% identical or similar to an amino acid sequence selected from the group consisting of SEQ ID NOs: 1,15 and 26. In another aspect, a Cas12a nuclease provided herein comprises an amino acid sequence at least 90% identical or similar to an amino acid sequence selected from the group consisting of SEQ ID NOs: 1,15 and 26. In another aspect, a Cas12a nuclease provided herein comprises an amino acid sequence at least 95% identical or similar to an amino acid sequence selected from the group consisting of SEQ ID NOs: 1, 15 and 26. In another aspect, a Cas12a nuclease provided herein comprises an amino acid sequence at least 96% identical or similar to an amino acid sequence selected from the group consisting of SEQ ID NOs: 1, 15 and 26. In another aspect, a Cas 12a nuclease provided herein comprises an amino acid sequence at least 97% identical or similar to an amino acid sequence selected from the group consisting of SEQ ID NOs: 1, 15 and 26. In another aspect, a Cas12a nuclease provided herein comprises an amino acid sequence at least 98% identical or similar to an amino acid sequence selected from the group consisting of SEQ ID NOs: 1, 15 and 26. In another aspect, a Cas12a nuclease provided herein comprises an amino acid sequence at least 99% identical or similar to an amino acid sequence selected from the group consisting of SEQ ID NOs: 1, 15 and 26. In another aspect, a Cas12a nuclease provided herein comprises an amino acid sequence 100% identical or similar to an amino acid sequence selected from the group consisting of SEQ ID NOs: 1, 15 and 26.

In an aspect, a Cas12a nuclease is encoded by a polynucleotide comprising a sequence at least 80% identical to a polynucleotide selected from the group consisting of SEQ ID NOs: 4 and 16.

In another aspect, a Cas12a nuclease is encoded by a polynucleotide comprising a sequence at least 85% identical to a polynucleotide selected from the group consisting of SEQ ID NOs: 4 and 16. In another aspect, a Cas12a nuclease is encoded by a polynucleotide comprising a sequence at least 90% identical to a polynucleotide selected from the group consisting of SEQ ID NOs: 4 and 16. In another aspect, a Cas12a nuclease is encoded by a polynucleotide comprising a sequence at least 95% identical to a polynucleotide selected from the group consisting of SEQ ID NOs: 4 and 16. In another aspect, a Cas12a nuclease is encoded by a polynucleotide comprising a sequence at least 96% identical to a polynucleotide selected from the group consisting of SEQ ID NOs: 4 and 16. In another aspect, a Cas12a nuclease is encoded by a polynucleotide comprising a sequence at least 97% identical to a polynucleotide selected from the group consisting of SEQ ID NOs: 4 and 16. In another aspect, a Cas12a nuclease is encoded by a polynucleotide comprising a sequence at least 98% identical to a polynucleotide selected from the group consisting of SEQ ID NOs: 4 and 16. In another aspect, a Cas12a nuclease is encoded by a polynucleotide comprising a sequence at least 99% identical to a polynucleotide selected from the group consisting of SEQ ID NOs: 4 and 16. In another aspect, a Cas12a nuclease is encoded by a polynucleotide comprising a sequence 100% identical to a polynucleotide selected from the group consisting of SEQ ID NOs: 4 and 16.

CasX is a type of class II CRISPR-Cas nuclease that has been identified in the bacterial phyla Deltaproteobacteria and Planctomycetes. Similar to Cas12a, CasX nucleases generate staggered cuts when cleaving a target nucleic acid molecule. However, unlike Cas12a, CasX nucleases require a crRNA and a tracrRNA, or a single-guide RNA, in order to target and cleave a target nucleic acid.

In an aspect, a CasX nuclease provided herein is a CasX nuclease from the phylum Deltaproteobacteria. In another aspect, a CasX nuclease provided herein is a CasX nuclease from the phylum Planctomycetes. Additional suitable CasX nucleases are those set forth in WO 2019/084148, which is incorporated by reference herein in its entirety.

In an aspect, a Cas12a nuclease or CasX nuclease provided herein can be expressed from a recombinant vector in vivo. In an aspect, a Cas12a nuclease or CasX nuclease provided herein can be expressed from a recombinant vector in vitro. In an aspect, a Cas12a nuclease or CasX nuclease provided herein can be expressed from a recombinant vector ex vivo. In an aspect, a Cas12a nuclease or CasX nuclease provided herein can be expressed from a nucleic acid molecule in vivo. In an aspect, a Cas12a nuclease or CasX nuclease provided herein can be expressed from a nucleic acid molecule in vitro. In an aspect, a Cas12a nuclease or CasX nuclease provided herein can be expressed from a nucleic acid molecule ex vivo.

As used herein, a “guide nucleic acid” refers to a nucleic acid that forms a complex with a CRISPR nuclease (e.g., without being limiting, Cas12a, CasX) and then guides the complex to a specific sequence in a target nucleic acid molecule, where the guide nucleic acid and the target nucleic acid molecule share complementary sequences. In an aspect, a ribonucleoprotein provided herein comprises at least one guide nucleic acid.

In an aspect, a guide nucleic acid comprises DNA. In another aspect, a guide nucleic acid comprises RNA. In an aspect, a guide nucleic acid comprises DNA, RNA, or a combination thereof. In an aspect, a guide nucleic acid is single-stranded. In another aspect, a guide nucleic acid is at least partially double-stranded.

When a guide nucleic acid comprises RNA, it can be referred to as a “guide RNA.” In another aspect, a guide nucleic acid comprises DNA and RNA. In another aspect, a guide nucleic acid is single-stranded. In another aspect, a guide nucleic acid is double-stranded. In a further aspect, a guide nucleic acid is partially double-stranded.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides.

In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence

complementarity to a target site. In an aspect, a guide nucleic acid comprises at least 75% sequence complementarity to a target site. In an aspect, a guide nucleic acid comprises at least 80% sequence complementarity to a target site. In an aspect, a guide nucleic acid comprises at least 85% sequence complementarity to a target site. In an aspect, a guide nucleic acid comprises at least 90% sequence complementarity to a target site. In an aspect, a guide nucleic acid comprises at least 91% sequence complementarity to a target site. In an aspect, a guide nucleic acid comprises at least 92% sequence complementarity to a target site. In an aspect, a guide nucleic acid comprises at least 93% sequence complementarity to a target site. In an aspect, a guide nucleic acid comprises at least 94% sequence complementarity to a target site. In an aspect, a guide nucleic acid comprises at least 95% sequence complementarity to a target site. In an aspect, a guide nucleic acid comprises at least 96% sequence complementarity to a target site. In an aspect, a guide nucleic acid comprises at least 97% sequence complementarity to a target site. In an aspect, a guide nucleic acid comprises at least 98% sequence complementarity to a target site. In an aspect, a guide nucleic acid comprises at least 99% sequence complementarity to a target site. In an aspect, a guide nucleic acid comprises 100% sequence complementarity to a target site. In another aspect, a guide nucleic acid comprises between 70% and 100% sequence complementarity to a target site. In another aspect, a guide nucleic acid comprises between 80% and 100% sequence complementarity to a target site. In another aspect, a guide nucleic acid comprises between 90% and 100% sequence complementarity to a target site.

In an aspect, a guide nucleic acid is capable of hybridizing to a target site.

As noted above, some RNA-guided CRISPR nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site, where CasX can cleave the target site. In other embodiments, the crRNA and tracrRNA are provided as separate nucleic acid molecules.

In an aspect, a guide nucleic acid comprises a crRNA. In another aspect, a guide nucleic acid comprises a tracrRNA. In a further aspect, a guide nucleic acid comprises an sgRNA.

In an aspect, a guide nucleic acid provided herein can be expressed from a recombinant vector in vivo. In an aspect, a guide nucleic acid provided herein can be expressed from a recombinant vector in vitro. In an aspect, a guide nucleic acid provided herein can be expressed from a recombinant vector ex vivo. In an aspect, a guide nucleic acid provided herein can be expressed from a nucleic acid molecule in vivo. In an aspect, a guide nucleic acid provided herein can be expressed from a nucleic acid molecule in vitro. In an aspect, a guide nucleic acid provided herein can be expressed from a nucleic acid molecule ex vivo. In another aspect, a guide nucleic acid provided herein can be synthetically synthesized.

Linkers are short amino acid sequences used to join two or more proteins or protein domains into one larger protein complex. Linkers do not interfere with the native function of any protein, or protein domain, to which they are attached.

In an aspect, a linker is positioned between an amino acid sequence encoding a first nuclease and an amino acid sequence encoding a second nuclease. In an aspect, a first nuclease is selected from the group consisting of a Cas12a nuclease, a CasX nuclease, a Cas9 nuclease, a meganuclease, a zinc-finger nuclease, a transcription activator-like nuclease, and a HUH endonuclease. In an aspect, a second nuclease is selected from the group consisting of a Cas 12a nuclease, a CasX nuclease, a Cas9 nuclease, a meganuclease, a zinc-finger nuclease, a transcription activator-like nuclease, and a HUH endonuclease.

In an aspect, a linker is positioned between an amino acid sequence encoding a nuclease and an amino acid sequence encoding a functional domain. In an aspect, the nuclease is selected from the group consisting of a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo), an RNA-guided nuclease, such as a CRISPR associated nuclease (non-limiting examples of CRISPR associated nucleases include Cas1, Cas1B, Cas2, Cas3, Cas4, Cas5, Cas6, Cas7, Cas8, Cas9 (also known as Csn1 and Csx12), Cas10, Cas12a (also known as Cpf1), Csyl, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, homologs thereof, or modified versions thereof). In an aspect, the functional domain is selected from the group consisting of a deaminase, a uracil DNA glycosylase (UGI), a transcriptional activator, a recombinase, a transposase, a helicase and a methylase. In some embodiments, the deaminase is a cytidine deaminase. In some embodiments, the deaminase is an adenine deaminase. In some embodiments, the deaminase is an APOPEC deaminase. In some embodiments, the deaminase is an activation-induced cytidine deaminase (AID). Non-limiting examples of recombinases include a tyrosine recombinase attached to a linker provided herein is selected from the group consisting of a Cre recombinase, a Gin recombinase, a Flp recombinase, and a Tnp1 recombinase. In another aspect, a serine recombinase attached to a linker provided herein is selected from the group consisting of a PhiC31 integrase, an R4 integrase, and a TP-901 integrase. In another aspect, a DNA transposase attached to a linker provided herein is selected from the group consisting of a TALE-piggyBac and TALE-Mutator.

In an aspect, a linker is positioned between a first amino acid sequence encoding a Cas12a nuclease and a second amino acid sequence encoding a HUH endonuclease. In another aspect, a linker is positioned between a first amino acid sequence encoding a CasX nuclease and a second amino acid sequence encoding a HUH endonuclease.

In an aspect, a linker is positioned on the 5′ end of a Cas12a nuclease. In another aspect, a linker is positioned on the 3′ end of a Cas12a nuclease. In another aspect, a linker is positioned on the 5′ end of a CasX nuclease. In another aspect, a linker is positioned on the 3′ end of a CasX nuclease. In another aspect, a linker is positioned on the 5′ end of a HUH endonuclease. In another aspect, a linker is positioned on the 3′ end of a HUH endonuclease.

In an aspect, a linker comprises at least 5 amino acids. In another aspect, a linker comprises at least 10 amino acids. In another aspect, a linker comprises at least 15 amino acids. In another aspect, a linker comprises at least 20 amino acids. In another aspect, a linker comprises at least 25 amino acids. In another aspect, a linker comprises at least 30 amino acids. In another aspect, a linker comprises at least 40 amino acids. In another aspect, a linker comprises at least 50 amino acids.

In an aspect, a linker comprises between 5 amino acids and 50 amino acids. In another aspect, a linker comprises between 5 amino acids and 40 amino acids. In another aspect, a linker comprises between 5 amino acids and 30 amino acids. In another aspect, a linker comprises between 5 amino acids and 20 amino acids. In another aspect, a linker comprises between 10 amino acids and 50 amino acids. In another aspect, a linker comprises between 10 amino acids and 40 amino acids. In another aspect, a linker comprises between 10 amino acids and 30 amino acids. In another aspect, a linker comprises between 10 amino acids and 20 amino acids.

In an aspect, a linker comprises 1 amino acid. In another aspect, a linker comprises 2 amino acids. In another aspect, a linker comprises 3 amino acids. In another aspect, a linker comprises 4 amino acids. In an aspect, a linker comprises 5 amino acids. In another aspect, a linker comprises 6 amino acids. In another aspect, a linker comprises 7 amino acids. In another aspect, a linker comprises 8 amino acids. In another aspect, a linker comprises 9 amino acids. In another aspect, a linker comprises 10 amino acids. In another aspect, a linker comprises 11 amino acids. In another aspect, a linker comprises 12 amino acids. In another aspect, a linker comprises 13 amino acids. In another aspect, a linker comprises 14 amino acids. In another aspect, a linker comprises 15 amino acids. In another aspect, a linker comprises 16 amino acids. In another aspect, a linker comprises 17 amino acids. In another aspect, a linker comprises 18 amino acids. In another aspect, a linker comprises 19 amino acids. In another aspect, a linker comprises 20 amino acids. In another aspect, a linker comprises 21 amino acids. In another aspect, a linker comprises 22 amino acids. In another aspect, a linker comprises 23 amino acids. In another aspect, a linker comprises 24 amino acids. In another aspect, a linker comprises 25 amino acids. In another aspect, a linker comprises 26 amino acids. In another aspect, a linker comprises 27 amino acids. In another aspect, a linker comprises 28 amino acids. In another aspect, a linker comprises 29 amino acids. In another aspect, a linker comprises 30 amino acids.

In an aspect, this disclosure provides an isolated polypeptide comprising an amino acid sequence at least 70% identical or similar to SEQ ID NO: 3. In an aspect, this disclosure provides an isolated polypeptide comprising an amino acid sequence at least 75% identical or similar to SEQ ID NO: 3. In an aspect, this disclosure provides an isolated polypeptide comprising an amino acid sequence at least 80% identical or similar to SEQ ID NO: 3. In an aspect, this disclosure provides an isolated polypeptide comprising an amino acid sequence at least 85% identical or similar to SEQ ID NO: 3. In an aspect, this disclosure provides an isolated polypeptide comprising an amino acid sequence at least 90% identical or similar to SEQ ID NO: 3. In an aspect, this disclosure provides an isolated polypeptide comprising an amino acid sequence at least 91% identical or similar to SEQ ID NO: 3. In an aspect, this disclosure provides an isolated polypeptide comprising an amino acid sequence at least 92% identical or similar to SEQ ID NO: 3. In an aspect, this disclosure provides an isolated polypeptide comprising an amino acid sequence at least 93% identical or similar to SEQ ID NO: 3. In an aspect, this disclosure provides an isolated polypeptide comprising an amino acid sequence at least 94% identical or similar to SEQ ID NO: 3. In an aspect, this disclosure provides an isolated polypeptide comprising an amino acid sequence at least 95% identical or similar to SEQ ID NO: 3. In an aspect, this disclosure provides an isolated polypeptide comprising an amino acid sequence at least 96% identical or similar to SEQ ID NO: 3. In an aspect, this disclosure provides an isolated polypeptide comprising an amino acid sequence at least 97% identical or similar to SEQ ID NO: 3. In an aspect, this disclosure provides an isolated polypeptide comprising an amino acid sequence at least 98% identical or similar to SEQ ID NO: 3. In an aspect, this disclosure provides an isolated polypeptide comprising an amino acid sequence at least 99% identical or similar to SEQ ID NO: 3. In an aspect, this disclosure provides an isolated polypeptide comprising an amino acid sequence 100% identical or similar to SEQ ID NO: 3.

In an aspect, a linker comprises an amino acid sequence at least 70% identical or similar to SEQ ID NO: 3. In another aspect, a linker comprises an amino acid sequence at least 75% identical or similar to SEQ ID NO: 3. In an aspect, a linker comprises an amino acid sequence at least 80% identical or similar to SEQ ID NO: 3. In another aspect, a linker comprises an amino acid sequence at least 85% identical or similar to SEQ ID NO: 3. In another aspect, a linker comprises an amino acid sequence at least 90% identical or similar to SEQ ID NO: 3. In another aspect, a linker comprises an amino acid sequence at least 91% identical or similar to SEQ ID NO: 3. In another aspect, a linker comprises an amino acid sequence at least 92% identical or similar to SEQ ID NO: 3. In another aspect, a linker comprises an amino acid sequence at least 93% identical or similar to SEQ ID NO: 3. In another aspect, a linker comprises an amino acid sequence at least 94% identical or similar to SEQ ID NO: 3. In another aspect, a linker comprises an amino acid sequence at least 95% identical or similar to SEQ ID NO: 3. In another aspect, a linker comprises an amino acid sequence at least 96% identical or similar to SEQ ID NO: 3. In another aspect, a linker comprises an amino acid sequence at least 97% identical or similar to SEQ ID NO: 3. In another aspect, a linker comprises an amino acid sequence at least 98% identical or similar to SEQ ID NO: 3. In another aspect, a linker comprises an amino acid sequence at least 99% identical or similar to SEQ ID NO: 3. In another aspect, a linker comprises an amino acid sequence 100% identical or similar to SEQ ID NO: 3.

In an aspect, a linker provided herein comprises SEQ ID NO: 3.

In an aspect, a polynucleotide encoding a linker comprises a polynucleotide sequence at least 70% identical to SEQ ID NO: 6. In another aspect, a polynucleotide encoding a linker comprises a polynucleotide sequence at least 75% identical to SEQ ID NO: 6. In another aspect, a polynucleotide encoding a linker comprises a polynucleotide sequence at least 80% identical to SEQ ID NO: 6. In another aspect, a polynucleotide encoding a linker comprises a polynucleotide sequence at least 85% identical to SEQ ID NO: 6. In another aspect, a polynucleotide encoding a linker comprises a polynucleotide sequence at least 90% identical to SEQ ID NO: 6. In another aspect, a polynucleotide encoding a linker comprises a polynucleotide sequence at least 91% identical to SEQ ID NO: 6. In another aspect, a polynucleotide encoding a linker comprises a polynucleotide sequence at least 92% identical to SEQ ID NO: 6. In another aspect, a polynucleotide encoding a linker comprises a polynucleotide sequence at least 93% identical to SEQ ID NO: 6. In another aspect, a polynucleotide encoding a linker comprises a polynucleotide sequence at least 94% identical to SEQ ID NO: 6. In another aspect, a polynucleotide encoding a linker comprises a polynucleotide sequence at least 95% identical to SEQ ID NO: 6. In another aspect, a polynucleotide encoding a linker comprises a polynucleotide sequence at least 96% identical to SEQ ID NO: 6. In another aspect, a polynucleotide encoding a linker comprises a polynucleotide sequence at least 97% identical to SEQ ID NO: 6. In another aspect, a polynucleotide encoding a linker comprises a polynucleotide sequence at least 98% identical to SEQ ID NO: 6. In another aspect, a polynucleotide encoding a linker comprises a polynucleotide sequence at least 99% identical to SEQ ID NO: 6. In another aspect, a polynucleotide encoding a linker comprises a polynucleotide sequence 100% identical to SEQ ID NO: 6.

Amino acids comprise a carboxylic acid (COOH) group and an amino group (NH₂) bound to a carbon atom, and each amino acid further comprises a variable R group. Based on the properties of the R group, amino acids can be characterized into groups of hydrophobic (non-polar) and hydrophilic (polar) amino acids.

Hydrophobic amino acids include glycine, alanine, valine, leucine, isoleucine, methionine, proline, phenylalanine, and tryptophan. Hydrophilic amino acids include tyrosine, serine, threonine, cysteine, glutamine, asparagine, glutamic acid, aspartic acid, lysine, histidine, and arginine.

In an aspect, the amino acid sequence of a linker comprises at least 5% hydrophobic amino acid residues. In another aspect, the amino acid sequence of a linker comprises at least 10% hydrophobic amino acid residues. In another aspect, the amino acid sequence of a linker comprises at least 15% hydrophobic amino acid residues. In another aspect, the amino acid sequence of a linker comprises at least 20% hydrophobic amino acid residues. In another aspect, the amino acid sequence of a linker comprises at least 25% hydrophobic amino acid residues. In another aspect, the amino acid sequence of a linker comprises at least 30% hydrophobic amino acid residues. In another aspect, the amino acid sequence of a linker comprises at least 35% hydrophobic amino acid residues. In another aspect, the amino acid sequence of a linker comprises at least 40% hydrophobic amino acid residues. In another aspect, the amino acid sequence of a linker comprises at least 45% hydrophobic amino acid residues. In another aspect, the amino acid sequence of a linker comprises at least 50% hydrophobic amino acid residues. In another aspect, the amino acid sequence of a linker comprises at least 55% hydrophobic amino acid residues. In another aspect, the amino acid sequence of a linker comprises at least 60% hydrophobic amino acid residues. In another aspect, the amino acid sequence of a linker comprises at least 65% hydrophobic amino acid residues. In another aspect, the amino acid sequence of a linker comprises at least 70% hydrophobic amino acid residues. In another aspect, the amino acid sequence of a linker comprises at least 75% hydrophobic amino acid residues. In another aspect, the amino acid sequence of a linker comprises at least 80% hydrophobic amino acid residues. In another aspect, the amino acid sequence of a linker comprises at least 85% hydrophobic amino acid residues. In another aspect, the amino acid sequence of a linker comprises at least 90% hydrophobic amino acid residues. In another aspect, the amino acid sequence of a linker comprises at least 95% hydrophobic amino acid residues.

In an aspect, the amino acid sequence of a linker comprises between 5% and 90% hydrophobic amino acid residues. In another aspect, the amino acid sequence of a linker comprises between 5% and 75% hydrophobic amino acid residues. In another aspect, the amino acid sequence of a linker comprises between 5% and 50% hydrophobic amino acid residues. In another aspect, the amino acid sequence of a linker comprises between 5% and 25% hydrophobic amino acid residues. In another aspect, the amino acid sequence of a linker comprises between 25% and 75% hydrophobic amino acid residues. In another aspect, the amino acid sequence of a linker comprises between 50% and 75% hydrophobic amino acid residues. In another aspect, the amino acid sequence of a linker comprises between 10% and 75% hydrophobic amino acid residues. In another aspect, the amino acid sequence of a linker comprises between 10% and 90% hydrophobic amino acid residues.

In an aspect, at least 10% of the amino acid sequence of a linker is comprised of any combination of glycine, threonine, and serine. In another aspect, at least 15% of the amino acid sequence of a linker is comprised of any combination of glycine, threonine, and serine. In another aspect, at least 20% of the amino acid sequence of a linker is comprised of any combination of glycine, threonine, and serine. In another aspect, at least 25% of the amino acid sequence of a linker is comprised of any combination of glycine, threonine, and serine. In another aspect, at least 30% of the amino acid sequence of a linker is comprised of any combination of glycine, threonine, and serine. In another aspect, at least 35% of the amino acid sequence of a linker is comprised of any combination of glycine, threonine, and serine. In another aspect, at least 40% of the amino acid sequence of a linker is comprised of any combination of glycine, threonine, and serine. In another aspect, at least 45% of the amino acid sequence of a linker is comprised of any combination of glycine, threonine, and serine. In another aspect, at least 50% of the amino acid sequence of a linker is comprised of any combination of glycine, threonine, and serine. In another aspect, at least 55% of the amino acid sequence of a linker is comprised of any combination of glycine, threonine, and serine. In another aspect, at least 60% of the amino acid sequence of a linker is comprised of any combination of glycine, threonine, and serine. In another aspect, at least 65% of the amino acid sequence of a linker is comprised of any combination of glycine, threonine, and serine. In another aspect, at least 70% of the amino acid sequence of a linker is comprised of any combination of glycine, threonine, and serine. In another aspect, at least 75% of the amino acid sequence of a linker is comprised of any combination of glycine, threonine, and serine. In another aspect, at least 80% of the amino acid sequence of a linker is comprised of any combination of glycine, threonine, and serine. In another aspect, at least 85% of the amino acid sequence of a linker is comprised of any combination of glycine, threonine, and serine. In another aspect, at least 90% of the amino acid sequence of a linker is comprised of any combination of glycine, threonine, and serine. In another aspect, at least 95% of the amino acid sequence of a linker is comprised of any combination of glycine, threonine, and serine.

In an aspect, between 10% and 95% of the amino acid sequence of a linker is comprised of any combination of glycine, threonine and serine. In another aspect, between 10% and 75% of the amino acid sequence of a linker is comprised of any combination of glycine, threonine and serine. In another aspect, between 10% and 50% of the amino acid sequence of a linker is comprised of any combination of glycine, threonine and serine. In another aspect, between 10% and 25% of the amino acid sequence of a linker is comprised of any combination of glycine, threonine and serine. In another aspect, between 25% and 75% of the amino acid sequence of a linker is comprised of any combination of glycine, threonine and serine. In another aspect, between 50% and 75% of the amino acid sequence of a linker is comprised of any combination of glycine, threonine and serine.

At physiological pH (e.g., 7.4), some amino acids have electrically charged R groups. For example, arginine, histidine, and lysine are positively charged amino acids; serine, threonine, asparagine, and glutamine are uncharged amino acids; and aspartic acid and glutamic acid are negatively charged amino acids.

In an aspect, the amino acid sequence of a linker comprises at least 5% negatively charged amino acid residues. In another aspect, the amino acid sequence of a linker comprises at least 10% negatively charged amino acid residues. In another aspect, the amino acid sequence of a linker comprises at least 15% negatively charged amino acid residues. In another aspect, the amino acid sequence of a linker comprises at least 20% negatively charged amino acid residues. In another aspect, the amino acid sequence of a linker comprises at least 25% negatively charged amino acid residues. In another aspect, the amino acid sequence of a linker comprises at least 30% negatively charged amino acid residues. In another aspect, the amino acid sequence of a linker comprises at least 35% negatively charged amino acid residues. In another aspect, the amino acid sequence of a linker comprises at least 40% negatively charged amino acid residues. In another aspect, the amino acid sequence of a linker comprises at least 45% negatively charged amino acid residues. In another aspect, the amino acid sequence of a linker comprises at least 50% negatively charged amino acid residues. In another aspect, the amino acid sequence of a linker comprises at least 55% negatively charged amino acid residues. In another aspect, the amino acid sequence of a linker comprises at least 60% negatively charged amino acid residues. In another aspect, the amino acid sequence of a linker comprises at least 65% negatively charged amino acid residues. In another aspect, the amino acid sequence of a linker comprises at least 70% negatively charged amino acid residues. In another aspect, the amino acid sequence of a linker comprises at least 75% negatively charged amino acid residues. In another aspect, the amino acid sequence of a linker comprises at least 80% negatively charged amino acid residues. In another aspect, the amino acid sequence of a linker comprises at least 85% negatively charged amino acid residues. In another aspect, the amino acid sequence of a linker comprises at least 90% negatively charged amino acid residues. In another aspect, the amino acid sequence of a linker comprises at least 95% negatively charged amino acid residues.

In an aspect, the amino acid sequence of a linker comprises between 5% and 90% negatively charged amino acid residues. In another aspect, the amino acid sequence of a linker comprises between 5% and 75% negatively charged amino acid residues. In another aspect, the amino acid sequence of a linker comprises between 5% and 50% negatively charged amino acid residues. In another aspect, the amino acid sequence of a linker comprises between 5% and 25% negatively charged amino acid residues. In another aspect, the amino acid sequence of a linker comprises between 25% and 75% negatively charged amino acid residues. In another aspect, the amino acid sequence of a linker comprises between 50% and 75% negatively charged amino acid residues. In another aspect, the amino acid sequence of a linker comprises between 10% and 75% negatively charged amino acid residues. In another aspect, the amino acid sequence of a linker comprises between 10% and 90% negatively charged amino acid residues.

In an aspect, a linker provided herein can be expressed from a recombinant vector in vivo. In an aspect, a linker provided herein can be expressed from a recombinant vector in vitro. In an aspect, a linker provided herein can be expressed from a recombinant vector ex vivo. In an aspect, a linker provided herein can be expressed from a nucleic acid molecule in vivo. In an aspect, a linker provided herein can be expressed from a nucleic acid molecule in vitro. In an aspect, a linker provided herein can be expressed from a nucleic acid molecule ex vivo.

HUH endonucleases contain a characteristic motif comprising a first histidine residue (H), a hydrophobic amino acid residue (U), and a second histidine residue (H). HUH endonucleases are known from archaeans, bacteria, and eukaryotes. Endogenous HUH endonucleases participate in cellular processes involving a transition from double-stranded to single-stranded DNA, such as rolling-circle replication in viruses and bacterial plasmid conjugation. The HUH endonuclease first nicks single-stranded DNA at a specific sequence at the origin of replication (ori) followed by formation of a covalent phosphotyrosine intermediate, whereby the 5′ end of the DNA strand becomes linked to a specific tyrosine in the HUH-protein. While the phosphotyrosine linkage is an intermediate in vivo, purified HUH-proteins are able to form stable covalent bonds in vitro with synthetic oligonucleotides bearing their ori sequence.

In an aspect, a HUH endonuclease hybridizes to an origin of replication (ori)

sequence.

In an aspect, a HUH endonuclease is porcine circovirus2 (PCV) HUH endonuclease. In another aspect, a HUH endonuclease is a duck circovirus (DCV) HUH endonuclease. In another aspect, a HUH endonuclease is a faba bean necrosis yellow virus (FBNYV) HUH endonuclease. In another aspect, a HUH endonuclease is a Streptococcus agalactiae replication protein RepB (RepB). In another aspect, a HUH endonuclease is a Fructobacillus tropaeoli RepB (RepBm). In another aspect, a HUH endonuclease is an Escherichia coli conjugation protein TraI (Tral). In another aspect, a HUH endonuclease is an E. coli mobilization protein A (mMobA). In another aspect, a HUH endonuclease is a Staphylococcus aureus nicking enzyme (NES).

In an aspect, a HUH endonuclease is selected from the group consisting of a FBNYV HUH endonuclease, a PCV HUH endonuclease, a DCV HUH endonuclease, a RepB, a RepBm, a Tral, a mMobA, and a NES.

In an aspect, a FBNYV HUH endonuclease comprises an amino acid sequence at least 80% identical or similar to the amino acid sequence of SEQ ID NO: 2. In another aspect, a FBNYV HUH endonuclease comprises an amino acid sequence at least 85% identical or similar to the amino acid sequence of SEQ ID NO: 2. In another aspect, a FBNYV HUH endonuclease comprises an amino acid sequence at least 90% identical or similar to the amino acid sequence of SEQ ID NO: 2. In another aspect, a FBNYV HUH endonuclease comprises an amino acid sequence at least 95% identical or similar to the amino acid sequence of SEQ ID NO: 2. In another aspect, a FBNYV HUH endonuclease comprises an amino acid sequence at least 96% identical or similar to the amino acid sequence of SEQ ID NO: 2. In another aspect, a FBNYV HUH endonuclease comprises an amino acid sequence at least 97% identical or similar to the amino acid sequence of SEQ ID NO: 2. In another aspect, a FBNYV HUH endonuclease comprises an amino acid sequence at least 98% identical or similar to the amino acid sequence of SEQ ID NO: 2. In another aspect, a FBNYV HUH endonuclease comprises an amino acid sequence at least 99% identical or similar to the amino acid sequence of SEQ ID NO: 2. In another aspect, a FBNYV HUH endonuclease comprises an amino acid sequence 100% identical or similar to the amino acid sequence of SEQ ID NO: 2.

In an aspect, a PCV HUH endonuclease comprises an amino acid sequence at least 80% identical or similar to the amino acid sequence of SEQ ID NO: 14. In another aspect, a PCV HUH endonuclease comprises an amino acid sequence at least 85% identical or similar to the amino acid sequence of SEQ ID NO: 14. In another aspect, a PCV HUH endonuclease comprises an amino acid sequence at least 90% identical or similar to the amino acid sequence of SEQ ID NO: 14. In another aspect, a PCV HUH endonuclease comprises an amino acid sequence at least 95% identical or similar to the amino acid sequence of SEQ ID NO: 14. In another aspect, a PCV HUH endonuclease comprises an amino acid sequence at least 96% identical or similar to the amino acid sequence of SEQ ID NO: 14. In another aspect, a PCV HUH endonuclease comprises an amino acid sequence at least 97% identical or similar to the amino acid sequence of SEQ ID NO: 14. In another aspect, a PCV HUH endonuclease comprises an amino acid sequence at least 98% identical or similar to the amino acid sequence of SEQ ID NO: 14. In another aspect, a PCV HUH endonuclease comprises an amino acid sequence at least 99% identical or similar to the amino acid sequence of SEQ ID NO: 14. In another aspect, a PCV HUH endonuclease comprises an amino acid sequence 100% identical or similar to the amino acid sequence of SEQ ID NO: 14.

In an aspect, a HUH endonuclease comprises an amino acid sequence at least 80% identical or similar to an amino acid sequence selected from the group consisting of SEQ ID NOs: 2 and 14. In an aspect, a HUH endonuclease comprises an amino acid sequence at least 85% identical or similar to an amino acid sequence selected from the group consisting of SEQ ID NOs: 2 and 14. In an aspect, a HUH endonuclease comprises an amino acid sequence at least 90% identical or similar to an amino acid sequence selected from the group consisting of SEQ ID NOs: 2 and 14. In an aspect, a HUH endonuclease comprises an amino acid sequence at least 95% identical or similar to an amino acid sequence selected from the group consisting of SEQ ID NOs: 2 and 14. In an aspect, a HUH endonuclease comprises an amino acid sequence at least 96% identical or similar to an amino acid sequence selected from the group consisting of SEQ ID NOs: 2 and 14. In an aspect, a HUH endonuclease comprises an amino acid sequence at least 97% identical or similar to an amino acid sequence selected from the group consisting of SEQ ID NOs: 2 and 14. In an aspect, a HUH endonuclease comprises an amino acid sequence at least 98% identical or similar to an amino acid sequence selected from the group consisting of SEQ ID NOs: 2 and 14. In an aspect, a HUH endonuclease comprises an amino acid sequence at least 99% identical or similar to an amino acid sequence selected from the group consisting of SEQ ID NOs: 2 and 14. In an aspect, a HUH endonuclease comprises an amino acid sequence 100% identical or similar to an amino acid sequence selected from the group consisting of SEQ ID NOs: 2 and 14.

In an aspect, a FBNYV HUH endonuclease is encoded by a nucleic acid sequence at least 80% identical to the nucleic acid sequence of SEQ ID NO: 5. In another aspect, a FBNYV HUH endonuclease is encoded by a nucleic acid sequence at least 85% identical to the nucleic acid sequence of SEQ ID NO: 5. In another aspect, a FBNYV HUH endonuclease is encoded by a nucleic acid sequence at least 90% identical to the nucleic acid sequence of SEQ ID NO: 5. In another aspect, a FBNYV HUH endonuclease is encoded by a nucleic acid sequence at least 95% identical to the nucleic acid sequence of SEQ ID NO: 5. In another aspect, a FBNYV HUH endonuclease is encoded by a nucleic acid sequence at least 96% identical to the nucleic acid sequence of SEQ ID NO: 5. In another aspect, a FBNYV HUH endonuclease is encoded by a nucleic acid sequence at least 97% identical to the nucleic acid sequence of SEQ ID NO: 5. In another aspect, a FBNYV HUH endonuclease is encoded by a nucleic acid sequence at least 98% identical to the nucleic acid sequence of SEQ ID NO: 5. In another aspect, a FBNYV HUH endonuclease is encoded by a nucleic acid sequence at least 99% identical to the nucleic acid sequence of SEQ ID NO: 5. In another aspect, a FBNYV HUH endonuclease is encoded by a nucleic acid sequence 100% identical to the nucleic acid sequence of SEQ ID NO: 5.

In an aspect, a PCV HUH endonuclease is encoded by a nucleic acid sequence at least 80% identical to the nucleic acid sequence of SEQ ID NO: 13. In another aspect, a PCV HUH endonuclease is encoded by a nucleic acid sequence at least 85% identical to the nucleic acid sequence of SEQ ID NO: 13. In another aspect, a PCV HUH endonuclease is encoded by a nucleic acid sequence at least 90% identical to the nucleic acid sequence of SEQ ID NO: 13. In another aspect, a PCV HUH endonuclease is encoded by a nucleic acid sequence at least 95% identical to the nucleic acid sequence of SEQ ID NO: 13. In another aspect, a PCV HUH endonuclease is encoded by a nucleic acid sequence at least 96% identical to the nucleic acid sequence of SEQ ID NO: 13. In another aspect, a PCV HUH endonuclease is encoded by a nucleic acid sequence at least 97% identical to the nucleic acid sequence of SEQ ID NO: 13. In another aspect, a PCV HUH endonuclease is encoded by a nucleic acid sequence at least 98% identical to the nucleic acid sequence of SEQ ID NO: 13. In another aspect, a PCV HUH endonuclease is encoded by a nucleic acid sequence at least 99% identical to the nucleic acid sequence of SEQ ID NO: 13. In another aspect, a PCV HUH endonuclease is encoded by a nucleic acid sequence 100% identical to the nucleic acid sequence of SEQ ID NO: 13.

In an aspect, a HUH endonuclease is encoded by a nucleic acid sequence at least 80% identical to a nucleic acid sequence selected from the group consisting of SEQ ID NOs: 5 and 13. In an aspect, a HUH endonuclease is encoded by a nucleic acid sequence at least 85% identical to a nucleic acid sequence selected from the group consisting of SEQ ID NOs: 5 and 13. In an aspect, a HUH endonuclease is encoded by a nucleic acid sequence at least 90% identical to a nucleic acid sequence selected from the group consisting of SEQ ID NOs: 5 and 13. In an aspect, a HUH endonuclease is encoded by a nucleic acid sequence at least 95% identical to a nucleic acid sequence selected from the group consisting of SEQ ID NOs: 5 and 13. In an aspect, a HUH endonuclease is encoded by a nucleic acid sequence at least 96% identical to a nucleic acid sequence selected from the group consisting of SEQ ID NOs: 5 and 13. In an aspect, a HUH endonuclease is encoded by a nucleic acid sequence at least 97% identical to a nucleic acid sequence selected from the group consisting of SEQ ID NOs: 5 and 13. In an aspect, a HUH endonuclease is encoded by a nucleic acid sequence at least 98% identical to a nucleic acid sequence selected from the group consisting of SEQ ID NOs: 5 and 13. In an aspect, a HUH endonuclease is encoded by a nucleic acid sequence at least 99% identical to a nucleic acid sequence selected from the group consisting of SEQ ID NOs: 5 and 13. In an aspect, a HUH endonuclease is encoded by a nucleic acid sequence 100% identical to a nucleic acid sequence selected from the group consisting of SEQ ID NOs: 5 and 13.

In an aspect, a HUH endonuclease provided herein can be expressed from a recombinant vector in vivo. In an aspect, a HUH endonuclease provided herein can be expressed from a recombinant vector in vitro. In an aspect, a HUH endonuclease provided herein can be expressed from a recombinant vector ex vivo. In an aspect, a HUH endonuclease provided herein can be expressed from a nucleic acid molecule in vivo. In an aspect, a HUH endonuclease provided herein can be expressed from a nucleic acid molecule in vitro. In an aspect, a HUH endonuclease provided herein can be expressed from a nucleic acid molecule ex vivo.

In an aspect, a ribonucleoprotein comprises an amino acid sequence at least 80% identical or similar to an amino acid selected from the group consisting of SEQ ID NOS: 20-23 and 25. In an aspect, a ribonucleoprotein comprises an amino acid sequence at least 85% identical or similar to an amino acid selected from the group consisting of SEQ ID NOs: 20-23 and 25. In an aspect, a ribonucleoprotein comprises an amino acid sequence at least 90% identical or similar to an amino acid selected from the group consisting of SEQ ID NOs: 20-23 and 25. In an aspect, a ribonucleoprotein comprises an amino acid sequence at least 95% identical or similar to an amino acid selected from the group consisting of SEQ ID NOs: 20-23 and 25. In an aspect, a ribonucleoprotein comprises an amino acid sequence at least 96% identical or similar to an amino acid selected from the group consisting of SEQ ID NOs: 20-23 and 25. In an aspect, a ribonucleoprotein comprises an amino acid sequence at least 97% identical or similar to an amino acid selected from the group consisting of SEQ ID NOs: 20-23 and 25. In an aspect, a ribonucleoprotein comprises an amino acid sequence at least 98% identical or similar to an amino acid selected from the group consisting of SEQ ID NOs: 20-23 and 25. In an aspect, a ribonucleoprotein comprises an amino acid sequence at least 99% identical or similar to an amino acid selected from the group consisting of SEQ ID NOs: 20-23 and 25. In an aspect, a ribonucleoprotein comprises an amino acid sequence 100% identical or similar to an amino acid selected from the group consisting of SEQ ID NOs: 20-23 and 25.

In an aspect, a recombinant nucleic acid encodes an amino acid sequence at least 80% identical or similar to an amino acid selected from the group consisting of SEQ ID NOs: 20-23 and 25. In an aspect, a recombinant nucleic acid encodes an amino acid sequence at least 85% identical or similar to an amino acid selected from the group consisting of SEQ ID NOs: 20-23 and 25. In an aspect, a recombinant nucleic acid encodes an amino acid sequence at least 90% identical or similar to an amino acid selected from the group consisting of SEQ ID NOs: 20-23 and 25. In an aspect, a recombinant nucleic acid encodes an amino acid sequence at least 95% identical or similar to an amino acid selected from the group consisting of SEQ ID NOs: 20-23 and 25. In an aspect, a recombinant nucleic acid encodes an amino acid sequence at least 96% identical or similar to an amino acid selected from the group consisting of SEQ ID NOs: 20-23 and 25. In an aspect, a recombinant nucleic acid encodes an amino acid sequence at least 97% identical or similar to an amino acid selected from the group consisting of SEQ ID NOs: 20-23 and 25. In an aspect, a recombinant nucleic acid encodes an amino acid sequence at least 98% identical or similar to an amino acid selected from the group consisting of SEQ ID NOs: 20-23 and 25. In an aspect, a recombinant nucleic acid encodes an amino acid sequence at least 99% identical or similar to an amino acid selected from the group consisting of SEQ ID NOS: 20-23 and 25. In an aspect, a recombinant nucleic acid encodes an amino acid sequence 100% identical or similar to an amino acid selected from the group consisting of SEQ ID NOs: 20-23 and 25.

In aspect, a recombinant nucleic acid comprises a polynucleotide sequence at least 80% identical to a sequence selected from the group consisting of SEQ ID Nos: 7, 17-19, and 24. In aspect, a recombinant nucleic acid comprises a polynucleotide sequence at least 85% identical to a sequence selected from the group consisting of SEQ ID Nos: 7, 17-19, and 24. In aspect, a recombinant nucleic acid comprises a polynucleotide sequence at least 90% identical to a sequence selected from the group consisting of SEQ ID Nos: 7, 17-19, and 24. In aspect, a recombinant nucleic acid comprises a polynucleotide sequence at least 95% identical to a sequence selected from the group consisting of SEQ ID Nos: 7, 17-19, and 24. In aspect, a recombinant nucleic acid comprises a polynucleotide sequence at least 96% identical to a sequence selected from the group consisting of SEQ ID Nos: 7, 17-19, and 24. In aspect, a recombinant nucleic acid comprises a polynucleotide sequence at least 97% identical to a sequence selected from the group consisting of SEQ ID Nos: 7, 17-19, and 24. In aspect, a recombinant nucleic acid comprises a polynucleotide sequence at least 98% identical to a sequence selected from the group consisting of SEQ ID Nos: 7, 17-19, and 24. In aspect, a recombinant nucleic acid comprises a polynucleotide sequence at least 99% identical to a sequence selected from the group consisting of SEQ ID Nos: 7, 17-19, and 24. In aspect, a recombinant nucleic acid comprises a polynucleotide sequence at least 100% identical to a sequence selected from the group consisting of SEQ ID Nos: 7,17-19, and 24.

As used herein, a “template nucleic acid molecule” refers to a nucleic acid molecule that comprises a nucleic acid sequence that is to be inserted into a target DNA molecule. In an aspect, a template nucleic acid molecule comprises single-stranded DNA. In another aspect, a template nucleic acid molecule comprises double-stranded DNA. In a further aspect, a template nucleic acid molecule comprises single-stranded RNA. In yet another aspect, a template nucleic acid molecule comprises double-stranded RNA. In another aspect, a template nucleic acid molecule comprises DNA and RNA.

In an aspect, a ribonucleoprotein comprises at least one template nucleic acid molecule. In another aspect, a ribonucleoprotein comprises at least two template nucleic acid molecules.

In an aspect, a template nucleic acid molecule comprises at least 10 nucleotides. In another aspect, a template nucleic acid molecule comprises at least 25 nucleotides. In another aspect, a template nucleic acid molecule comprises at least 50 nucleotides. In another aspect, a template nucleic acid molecule comprises at least 75 nucleotides. In another aspect, a template nucleic acid molecule comprises at least 100 nucleotides. In another aspect, a template nucleic acid molecule comprises at least 250 nucleotides. In another aspect, a template nucleic acid molecule comprises at least 500 nucleotides. In another aspect, a template nucleic acid molecule comprises at least 750 nucleotides. In another aspect, a template nucleic acid molecule comprises at least 1000 nucleotides. In another aspect, a template nucleic acid molecule comprises at least 2500 nucleotides.

In an aspect, a template nucleic acid molecule comprises between 10nucleotides and 2500 nucleotides. In another aspect, a template nucleic acid molecule comprises between 25 nucleotides and 2500 nucleotides. In another aspect, a template nucleic acid molecule comprises between 50 nucleotides and 2500 nucleotides. In another aspect, a template nucleic acid molecule comprises between 75 nucleotides and 2500 nucleotides. In another aspect, a template nucleic acid molecule comprises between 100 nucleotides and 2500 nucleotides. In another aspect, a template nucleic acid molecule comprises between 250 nucleotides and 2500 nucleotides. In another aspect, a template nucleic acid molecule comprises between 500 nucleotides and 2500 nucleotides. In another aspect, a template nucleic acid molecule comprises between 25 nucleotides and 1000 nucleotides. In another aspect, a template nucleic acid molecule comprises between 25 nucleotides and 500 nucleotides. In another aspect, a template nucleic acid molecule comprises between 25 nucleotides and 250 nucleotides.

As noted above, a HUH endonuclease first nicks single-stranded DNA at a specific sequence at the origin of replication (ori) followed by formation of a covalent phosphotyrosine intermediate, whereby the 5′ end of the DNA strand becomes linked to a specific tyrosine in the HUH-protein. While the phosphotyrosine linkage is an intermediate in vivo, purified HUH-proteins are able to form stable covalent bonds in vitro with synthetic oligonucleotides bearing their ori sequence.

In aspect, a template nucleic acid molecule comprises a nucleic acid sequence encoding an origin of replication (ori). In an aspect, an ori provided herein is capable of hybridizing to a HUH endonuclease. In an aspect, an ori comprises a nucleic acid sequence at least 80% identical to SEQ ID NO: 12. In another aspect, an ori comprises a nucleic acid sequence at least 85% identical to SEQ ID NO: 12. In another aspect, an ori comprises a nucleic acid sequence at least 90% identical to SEQ ID NO: 12. In another aspect, an ori comprises a nucleic acid sequence at least 95% identical to SEQ ID NO: 12. In another aspect, an ori comprises a nucleic acid sequence at least 96% identical to SEQ ID NO: 12. In another aspect, an ori comprises a nucleic acid sequence at least 97% identical to SEQ ID NO: 12. In another aspect, an ori comprises a nucleic acid sequence at least 98% identical to SEQ ID NO: 12. In another aspect, an ori comprises a nucleic acid sequence at least 99% identical to SEQ ID NO: 12. In another aspect, an ori comprises a nucleic acid sequence 100% identical to SEQ ID NO: 12. In an aspect, an ori comprises a nucleic acid sequence at least 80% identical to SEQ ID NO: 27. In another aspect, an ori comprises a nucleic acid sequence at least 85% identical to SEQ ID NO: 27. In another aspect, an ori comprises a nucleic acid sequence at least 90% identical to SEQ ID NO: 27. In another aspect, an ori comprises a nucleic acid sequence at least 95% identical to SEQ ID NO: 27. In another aspect, an ori comprises a nucleic acid sequence at least 96% identical to SEQ ID NO: 27. In another aspect, an ori comprises a nucleic acid sequence at least 97% identical to SEQ ID NO: 27. In another aspect, an ori comprises a nucleic acid sequence at least 98% identical to SEQ ID NO: 27. In another aspect, an ori comprises a nucleic acid sequence at least 99% identical to SEQ ID NO: 27. In another aspect, an ori comprises a nucleic acid sequence 100% identical to SEQ ID NO: 27. In an aspect, an ori comprises a nucleic acid sequence at least 80% identical to SEQ ID NO: 28. In another aspect, an ori comprises a nucleic acid sequence at least 85% identical to SEQ ID NO: 28. In another aspect, an ori comprises a nucleic acid sequence at least 90% identical to SEQ ID NO: 28. In another aspect, an ori comprises a nucleic acid sequence at least 95% identical to SEQ ID NO: 28. In another aspect, an ori comprises a nucleic acid sequence at least 96% identical to SEQ ID NO: 28. In another aspect, an ori comprises a nucleic acid sequence at least 97% identical to SEQ ID NO: 28. In another aspect, an ori comprises a nucleic acid sequence at least 98% identical to SEQ ID NO: 28. In another aspect, an ori comprises a nucleic acid sequence at least 99% identical to SEQ ID NO: 28. In another aspect, an ori comprises a nucleic acid sequence 100% identical to SEQ ID NO: 28.

In an aspect, an ori comprises at least 10 nucleotides. In an aspect, an ori comprises at least 15 nucleotides. In an aspect, an ori comprises at least 20 nucleotides. In an aspect, an ori comprises at least 25 nucleotides. In an aspect, an ori comprises at least 30 nucleotides. In an aspect, an ori comprises at least 40 nucleotides.

In an aspect, an ori is positioned on the 5′-end of a template nucleic acid molecule. In another aspect, an ori is positioned on the 3′-end of a template nucleic acid molecule.

In an aspect, a template nucleic acid molecule comprises a nucleic acid sequence encoding a gene of interest. As used herein, a “gene of interest” refers to a polynucleotide sequence encoding a protein or a non-protein-coding RNA molecule that is to be inserted into a target DNA molecule. In an aspect, a gene of interest encodes a protein. In another aspect, a gene of interest encodes a non-protein-coding RNA molecule.

Non-limiting examples of a non-protein-coding RNA molecule include a microRNA (miRNA), a miRNA precursor (pre-miRNA), a small interfering RNA (siRNA), a small RNA (18 to 26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA). In an aspect, a non-protein-coding RNA molecule comprises a miRNA. In an aspect, a non-protein-coding RNA molecule comprises a siRNA. In an aspect, a non-protein-coding RNA molecule comprises a ta-siRNA. In an aspect, a non-protein-coding RNA molecule is selected from the group consisting of a miRNA, a siRNA, and a ta-siRNA.

In an aspect, a gene of interest is exogenous to a target DNA molecule. In an aspect, a gene of interest replaces an endogenous gene in a target DNA molecule.

As used herein, a “target DNA molecule” refers to a selected DNA molecule or a selected sequence or region of a DNA molecule in which a modification (e.g., cleavage, site-directed integration) is desired.

As used herein, a “target region” refers to the portion of a target DNA molecule that is cleaved by a CRISPR nuclease. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target DNA molecule is single-stranded. In another aspect, a target DNA molecule is double-stranded.

In an aspect, a target DNA molecule comprises genomic DNA. In an aspect, a target DNA molecule is positioned within a nuclear genome. In an aspect, a target DNA molecule comprises chromosomal DNA. In an aspect, a target DNA molecule comprises plasmid DNA. In an aspect, a target DNA molecule is positioned within a plasmid. In an aspect, a target DNA molecule comprises mitochondrial DNA. In an aspect, a target DNA molecule is positioned within a mitochondrial genome. In an aspect, a target DNA molecule comprises plastid DNA. In an aspect, a target DNA molecule is positioned within a plastid genome. In an aspect, a target DNA molecule comprises chloroplast DNA. In an aspect, a target DNA molecule is positioned within a chloroplast genome. In an aspect, a target DNA molecule is positioned within a genome selected from the group consisting of a nuclear genome, a mitochondrial genome, and a plastid genome.

In an aspect, a target DNA molecule comprises genic DNA. As used herein, “genic DNA” refers to DNA that encodes one or more genes. In another aspect, a target DNA molecule comprises intergenic DNA. In contrast to genic DNA, “intergenic DNA” comprises noncoding DNA, and lacks DNA encoding a gene. In an aspect, intergenic DNA is positioned between two genes.

In an aspect, a target nucleic acid encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target DNA molecule is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

In an aspect, this disclosure provides a method of generating an edit in a target DNA molecule comprising contacting the target DNA molecule with a ribonucleoprotein, where the ribonucleoprotein comprises (a) a recombinant polypeptide comprising: (i) an amino acid sequence encoding a Cas12a nuclease; (ii) an amino acid sequence encoding a linker; and (iii) an amino acid sequence encoding a HUH endonuclease; (b) at least one guide nucleic acid; and (c) at least one template nucleic acid molecule, where the ribonucleoprotein edits the target DNA molecule. In an aspect, this disclosure provides a method of generating an edit in a target DNA molecule comprising contacting the target DNA molecule with a ribonucleoprotein, where the ribonucleoprotein comprises (a) a recombinant polypeptide comprising: (i) an amino acid sequence encoding a CasX nuclease; (ii) an amino acid sequence encoding a linker; and (iii) an amino acid sequence encoding a HUH endonuclease; (b) at least one guide nucleic acid; and (c) at least one template nucleic acid molecule, where the ribonucleoprotein edits the target DNA molecule. In an aspect, a method provided herein further comprises: (d) detecting the at least one edit in the target DNA molecule.

In an aspect, this disclosure provides a method of generating an edit in a target DNA molecule comprising providing to a cell: (a) a recombinant polypeptide comprising: (i) an amino acid sequence encoding a Cas12a nuclease; (ii) an amino acid sequence encoding a linker; and (iii) an amino acid sequence encoding a HUH endonuclease, or one or more nucleic acid molecules encoding the recombinant polypeptide; (b) at least one guide nucleic acid, or at least one nucleic acid molecule encoding the at least one guide nucleic acid; and (c) at least one template nucleic acid molecule, or at least one nucleic acid molecule encoding the at least one template nucleic acid molecule, where the recombinant polypeptide, the at least one guide nucleic acid, and the at least one template nucleic acid form a ribonucleoprotein, and where the ribonucleoprotein generates at least one edit in the target DNA molecule within the cell. In an aspect, this disclosure provides a method of generating an edit in a target DNA molecule comprising providing to a cell: (a) a recombinant polypeptide comprising: (i) an amino acid sequence encoding a CasX nuclease; (ii) an amino acid sequence encoding a linker; and (iii) an amino acid sequence encoding a HUH endonuclease, or one or more nucleic acid molecules encoding the recombinant polypeptide; (b) at least one guide nucleic acid, or at least one nucleic acid molecule encoding the at least one guide nucleic acid; and (c) at least one template nucleic acid molecule, or at least one nucleic acid molecule encoding the at least one template nucleic acid molecule, where the recombinant polypeptide, the at least one guide nucleic acid, and the at least one template nucleic acid form a ribonucleoprotein, and where the ribonucleoprotein generates at least one edit in the target DNA molecule within the cell. In an aspect, a method provided herein further comprises: (d) detecting the at least one edit in the target DNA molecule. In an aspect, the ribonucleoprotein forms within the cell. In an aspect, the ribonucleoprotein forms outside the cell. In an aspect, the ribonucleoprotein forms in vivo. In an aspect, the ribonucleoprotein forms in vitro.

In an aspect, an edit provided herein comprises a mutation. As used herein, a “mutation” refers to a non-naturally occurring alteration to a nucleic acid or amino acid sequence as compared to a naturally occurring reference nucleic acid or amino acid sequence from the same organism. It will be appreciated that, when identifying a mutation, the reference sequence should be from the same nucleic acid (e.g, gene, non-coding RNA) or amino acid (e.g, protein). In determining if an difference between two sequences comprises a mutation, it will be appreciated in the art that the comparison should not be made between homologous sequences of two different species or between homologous sequences of two different varieties of a single species. Rather, the comparison should be made between the edited sequence and the endogenous, non-edited (e.g., “wildtype”) sequence of the same organism.

In an aspect, a mutation comprises the insertion of at least one nucleotide or amino acid. In another aspect, a mutation comprises the deletion of at least one nucleotide or amino acid. In a further aspect, a mutation comprises the substitution of at least one nucleotide or amino acid. In still a further aspect, a mutation comprises the inversion of at least two nucleotides or amino acids. In another aspect, a mutation is selected from the group consisting of an insertion, a deletion, a substitution, and an inversion.

In an aspect, a mutation comprises a site-directed integration. In an aspect, a site-directed integration comprises the insertion of all or part of a template nucleic acid molecule into a target DNA molecule.

As used herein, “site-directed integration” refers to all, or a portion, of a desired sequence (e.g., template nucleic acid molecule) being inserted or integrated at a desired site or locus within the plant genome (e.g., target DNA molecule). The desired sequence can comprise a transgene or construct. In an aspect, a template nucleic acid molecule comprises one or two homology arms flanking the desired sequence to promote the targeted insertion event through homologous recombination and/or homology-directed repair.

Any site or locus within the genome of a plant, animal, fungi, or bacteria may potentially be chosen for site-directed integration of a transgene or construct of the present disclosure.

For site-directed integration, a double-strand break (DSB) or nick may first be made at a target DNA molecule via a RNA-guided CRISPR nuclease or ribonucleoprotein provided herein. In the presence of a template nucleic acid molecule, the DSB or nick can then be repaired by homologous recombination (HR) between the homology arm(s) of the template nucleic acid molecule and the target DNA molecule, or by non-homologous end joining (NHEJ), resulting in site-directed integration of all or part of the template nucleic acid molecule into the target DNA molecule to create the targeted insertion event at the site of the DSB or nick.

In an aspect, site-directed integration comprises the use of NHEJ repair mechanisms endogenous to a cell. In another aspect, site-directed integration comprises the use of HR repair mechanisms endogenous to a cell.

In an aspect, a mutation comprises the integration of at least 5 contiguous nucleotides of a template nucleic acid molecule into a target DNA molecule. In an aspect, a mutation comprises the integration of at least 10 contiguous nucleotides of a template nucleic acid molecule into a target DNA molecule. In an aspect, a mutation comprises the integration of at least 15 contiguous nucleotides of a template nucleic acid molecule into a target DNA molecule. In an aspect, a mutation comprises the integration of at least 20 contiguous nucleotides of a template nucleic acid molecule into a target DNA molecule. In an aspect, a mutation comprises the integration of at least 25 contiguous nucleotides of a template nucleic acid molecule into a target DNA molecule. In an aspect, a mutation comprises the integration of at least 50 contiguous nucleotides of a template nucleic acid molecule into a target DNA molecule. In an aspect, a mutation comprises the integration of at least 100 contiguous nucleotides of a template nucleic acid molecule into a target DNA molecule. In an aspect, a mutation comprises the integration of at least 250 contiguous nucleotides of a template nucleic acid molecule into a target DNA molecule. In an aspect, a mutation comprises the integration of at least 500 contiguous nucleotides of a template nucleic acid molecule into a target DNA molecule. In an aspect, a mutation comprises the integration of at least 1000 contiguous nucleotides of a template nucleic acid molecule into a target DNA molecule. In an aspect, a mutation comprises the integration of at least 2000 contiguous nucleotides of a template nucleic acid molecule into a target DNA molecule.

In an aspect, a mutation comprises the integration of between 5 contiguous nucleotides and 3500 contiguous nucleotides of a template nucleic acid molecule into a target DNA molecule. In an aspect, a mutation comprises the integration of between 5 contiguous nucleotides and 2500 contiguous nucleotides of a template nucleic acid molecule into a target DNA molecule. In an aspect, a mutation comprises the integration of between 5 contiguous nucleotides and 1500 contiguous nucleotides of a template nucleic acid molecule into a target DNA molecule. In an aspect, a mutation comprises the integration of between 5 contiguous nucleotides and 750 contiguous nucleotides of a template nucleic acid molecule into a target DNA molecule. In an aspect, a mutation comprises the integration of between 5 contiguous nucleotides and 500 contiguous nucleotides of a template nucleic acid molecule into a target DNA molecule. In an aspect, a mutation comprises the integration of between 5 contiguous nucleotides and 250 contiguous nucleotides of a template nucleic acid molecule into a target DNA molecule. In an aspect, a mutation comprises the integration of between 5 contiguous nucleotides and 150 contiguous nucleotides of a template nucleic acid molecule into a target DNA molecule. In an aspect, a mutation comprises the integration of between 25 contiguous nucleotides and 2500 contiguous nucleotides of a template nucleic acid molecule into a target DNA molecule. In an aspect, a mutation comprises the integration of between 25 contiguous nucleotides and 1500 contiguous nucleotides of a template nucleic acid molecule into a target DNA molecule. In an aspect, a mutation comprises the integration of between 25 contiguous nucleotides and 750 contiguous nucleotides of a template nucleic acid molecule into a target DNA molecule. In an aspect, a mutation comprises the integration of between 50 contiguous nucleotides and 2500 contiguous nucleotides of a template nucleic acid molecule into a target DNA molecule. In an aspect, a mutation comprises the integration of between 50 contiguous nucleotides and 1500 contiguous nucleotides of a template nucleic acid molecule into a target DNA molecule. In an aspect, a mutation comprises the integration of between 50 contiguous nucleotides and 750contiguous nucleotides of a template nucleic acid molecule into a target DNA molecule. In an aspect, a mutation comprises the integration of between 100 contiguous nucleotides and 2500 contiguous nucleotides of a template nucleic acid molecule into a target DNA molecule. In an aspect, a mutation comprises the integration of between 100 contiguous nucleotides and 1500 contiguous nucleotides of a template nucleic acid molecule into a target DNA molecule. In an aspect, a mutation comprises the integration of between 100 contiguous nucleotides and 750 contiguous nucleotides of a template nucleic acid molecule into a target DNA molecule.

In an aspect, a method provided herein comprises detecting an edit or a mutation in a target DNA molecule. Any method available in the art capable of detecting an edit or a mutation in a target DNA molecule. Without being limiting, suitable methods for detecting an edit or a mutation include a Southern blot, polymerase chain reaction (PCR), and nucleic acid sequencing.

Any method provided herein can involve transient transfection or stable transformation of a cell of interest (e.g., a eukaryotic cell, a prokaryotic cell). In an aspect, a nucleic acid molecule provided herein is stably transformed into a cell. In an aspect, a nucleic acid molecule provided herein is transiently transfected into a cell.

In an aspect, a nucleic acid molecule encoding Cas12a nuclease is stably transformed into a cell. In another aspect, a nucleic acid molecule encoding a Cas12a nuclease is transiently transfected into a cell. In another aspect, a Cas12a nuclease is transfected into a cell.

In an aspect, a nucleic acid molecule encoding a CasX nuclease is stably transformed into a cell. In another aspect, a nucleic acid molecule encoding a CasX nuclease is transiently transfected into a cell. In another aspect, a CasX nuclease is transfected into a cell.

In an aspect, a nucleic acid molecule encoding a linker is stably transformed into a cell. In another aspect, a nucleic acid molecule encoding a linker is transiently transfected into a cell. In another aspect, a linker is transfected into a cell.

In an aspect, a nucleic acid molecule encoding a HUH endonuclease is stably transformed into a cell. In another aspect, a nucleic acid molecule encoding a HUH endonuclease is transiently transfected into a cell. In another aspect, a HUH endonuclease is transfected into a cell.

In an aspect, a nucleic acid molecule encoding a FBNYV HUH endonuclease is stably transformed into a cell. In another aspect, a nucleic acid molecule encoding a FBNYV HUH endonuclease is transiently transfected into a cell. In another aspect, a FBNYV HUH endonuclease is transfected into a cell.

In an aspect, a nucleic acid molecule encoding a PCV HUH endonuclease is stably transformed into a cell. In another aspect, a nucleic acid molecule encoding a PCV HUH endonuclease is transiently transfected into a cell. In another aspect, a PCV HUH endonuclease is transfected into a cell.

In an aspect, a nucleic acid molecule encoding a guide nucleic acid is stably transformed into a cell. In another aspect, a nucleic acid molecule encoding a guide nucleic acid is transiently transfected into a cell. In another aspect, a guide nucleic acid is transfected into a cell.

In an aspect, a nucleic acid molecule encoding a template nucleic acid molecule is stably transformed into a cell. In another aspect, a nucleic acid molecule encoding a template nucleic acid molecule is transiently transfected into a cell. In another aspect, a template nucleic acid molecule is transfected into a cell.

In an aspect, a nucleic acid molecule encoding one or more components of a ribonucleoprotein is stably transformed into a cell. In another aspect, a nucleic acid molecule encoding one or more components of a ribonucleoprotein is transiently transfected into a cell. In another aspect, a ribonucleoprotein is transfected into a cell.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule, a protein, or a ribonucleoprotein via Agrobacterium-mediated transformation. In an aspect, a method comprises providing a cell with a nucleic acid molecule, a protein, or a ribonucleoprotein via polyethylene glycol-mediated transformation. In an aspect, a method comprises providing a cell with a nucleic acid molecule, a protein, or a ribonucleoprotein via biolistic transformation. In an aspect, a method comprises providing a cell with a nucleic acid molecule, a protein, or a ribonucleoprotein via liposome-mediated transfection. In an aspect, a method comprises providing a cell with a nucleic acid molecule, a protein, or a ribonucleoprotein via viral transduction. In an aspect, a method comprises providing a cell with a nucleic acid molecule, a protein, or a ribonucleoprotein via use of one or more delivery particles. In an aspect, a method comprises providing a cell with a nucleic acid molecule, a protein, or a ribonucleoprotein via microinjection. In an aspect, a method comprises providing a cell with a nucleic acid molecule, a protein, or a ribonucleoprotein via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

In an aspect, a protein is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

In an aspect, a ribonucleoprotein is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule or a protein are as used in WO 2014/093622 (PCT/US2013/074667). In an aspect, a method of providing a nucleic acid molecule or a protein to a cell comprises delivery via a delivery particle. In an aspect, a method of providing a nucleic acid molecule or a protein to a cell comprises delivery via a delivery vesicle. In an aspect, a delivery vesicle is selected from the group consisting of an exosome and a liposome. In an aspect, a method of providing a nucleic acid molecule or a protein to a cell comprises delivery via a viral vector. In an aspect, a viral vector is selected from the group consisting of an adenovirus vector, a lentivirus vector, and an adeno-associated viral vector. In another aspect, a method providing a nucleic acid molecule or a protein to a cell comprises delivery via a nanoparticle. In an aspect, a method providing a nucleic acid molecule or a protein to a cell comprises microinjection. In an aspect, a method providing a nucleic acid molecule or a protein to a cell comprises polycations. In an aspect, a method providing a nucleic acid molecule or a protein to a cell comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of proteins, nucleic acids, mutagens and ribonucleoproteins into a plant cell include agents that increase permeability of the exterior of the plant or that increase permeability of plant cells to oligonucleotides, polynucleotides, proteins, or ribonucleoproteins. Such agents to facilitate transfer of the composition into a plant cell include a chemical agent, or a physical agent, or combinations thereof. Chemical agents for conditioning includes (a) surfactants, (b) an organic solvents or an aqueous solutions or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e.g., plant-sourced oils, crop oils (such as those listed in the 9^{th} Compendium of Herbicide Adjuvants, publicly available on line at herbicide[dot]adjuvants[dot]com) can be used, e.g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e.g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et al. (2004) J. Am. Chem. Soc, 126(22):6850-6851, Liu et al. (2009) Nano Lett, 9(3):1007-1010, and Khodakovskaya et al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a prokaryotic cell. In another aspect, a transformed or transfected cell is a eukaryotic cell. In another aspect, a transformed or transfected cell is a plant cell. In another aspect, a transformed or transfected cell is an animal cell. In another aspect, a transformed or transfected cell is a fungus cell.

Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a stomata cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. No. 6,194,636 and 6,232,526 and U. S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

In an aspect, any nucleic acid molecule, polypeptide, or ribonucleoprotein provided herein is within a cell. In another aspect, any nucleic acid molecule, polypeptide, or ribonucleoprotein provided herein a prokaryotic cell. In an aspect, any nucleic acid molecule, polypeptide, or ribonucleoprotein provided herein a eukaryotic cell.

In an aspect, any cell provided herein is a host cell. In an aspect, a host cell comprises any ribonucleoprotein provided herein. In an aspect, a host cell comprises any polypeptide provided herein. In an aspect, a host cell comprises any nucleic acid molecule provided herein.

In an aspect, a host cell is selected from the group consisting of a plant cell, a bacterial cell, a mammalian cell, a fungal cell, an insect cell, an arachnid cell, a bird cell, a fish cell, a reptile cell, and an amphibian cell. In another aspect, a host plant cell is selected from the group consisting of a corn cell, a soybean cell, a cotton cell, a canola cell, a rice cell, a wheat cell, a sorghum cell, an alfalfa cell, a sugarcane cell, a millet cell, a tomato cell, a potato cell, and an algae cell. In another aspect, a host cell is an Escherichia coli cell.

In an aspect, a prokaryotic cell is a cell from a phylum selected from the group consisting of prokaryotic cell is a cell from a phylum selected from the group consisting of Acidobacteria, Actinobacteria, Aquificae, Armatimonadetes, Bacteroidetes, Caldiserica, Chlamydie, Chlorobi, Chloroflexi, Chrysiogenetes, Coprothermobacterota, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospirae, Planctomycetes, Proteobacteria, Spirochaetes, Synergistetes, Tenericutes, Thermodesulfobacteria, Thermotogae, and Verrucomicrobia. In another aspect, a prokaryotic cell is an Escherichia coli cell. In another aspect, a prokaryotic cell is selected from a genus selected from the group consisting of Escherichia, Agrobacterium, Rhizobium, Sinorhizobium, and Staphylococcus.

In an aspect, a eukaryotic cell is an ex vivo cell. In another aspect, a eukaryotic cell is a plant cell. In another aspect, a eukaryotic cell is a plant cell in culture. In another aspect, a eukaryotic cell is an angiosperm plant cell. In another aspect, a eukaryotic cell is a gymnosperm plant cell. In another aspect, a eukaryotic cell is a monocotyledonous plant cell. In another aspect, a eukaryotic cell is a dicotyledonous plant cell. In another aspect, a eukaryotic cell is a corn cell. In another aspect, a eukaryotic cell is a rice cell. In another aspect, a eukaryotic cell is a sorghum cell. In another aspect, a eukaryotic cell is a wheat cell. In another aspect, a eukaryotic cell is a canola cell. In another aspect, a eukaryotic cell is an alfalfa cell. In another aspect, a eukaryotic cell is a soybean cell. In another aspect, a eukaryotic cell is a cotton cell. In another aspect, a eukaryotic cell is a tomato cell. In another aspect, a eukaryotic cell is a potato cell. In a further aspect, a eukaryotic cell is a cucumber cell. In another aspect, a eukaryotic cell is a millet cell. In another aspect, a eukaryotic cell is a barley cell. In another aspect, a eukaryotic cell is a Brassica cell. In another aspect, a eukaryotic cell is a grass cell. In another aspect, a eukaryotic cell is a Setaria cell. In another aspect, a eukaryotic cell is an Arabidopsis cell. In a further aspect, a eukaryotic cell is an algae cell.

In one aspect, a plant cell is an epidermal cell. In another aspect, a plant cell is a stomata cell. In another aspect, a plant cell is a trichome cell. In another aspect, a plant cell is a root cell. In another aspect, a plant cell is a leaf cell. In another aspect, a plant cell is a callus cell. In another aspect, a plant cell is a protoplast cell. In another aspect, a plant cell is a pollen cell. In another aspect, a plant cell is an ovary cell. In another aspect, a plant cell is a floral cell. In another aspect, a plant cell is a meristematic cell. In another aspect, a plant cell is an endosperm cell. In another aspect, a plant cell does not comprise reproductive material and does not mediate the natural reproduction of the plant. In another aspect, a plant cell is a somatic plant cell.

Additional provided plant cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue.

In a further aspect, a eukaryotic cell is an animal cell. In another aspect, a eukaryotic cell is an animal cell in culture. In a further aspect, a eukaryotic cell is a human cell. In another aspect, a eukaryotic cell is not a human stem cell. In a further aspect, a eukaryotic cell is a human cell in culture. In a further aspect, a eukaryotic cell is a somatic human cell. In a further aspect, a eukaryotic cell is a cancer cell. In a further aspect, a eukaryotic cell is a mammal cell. In a further aspect, a eukaryotic cell is a mouse cell. In a further aspect, a eukaryotic cell is a pig cell. In a further aspect, a eukaryotic cell is a bovid cell. In a further aspect, a eukaryotic cell is a bird cell. In a further aspect, a eukaryotic cell is a reptile cell. In a further aspect, a eukaryotic cell is an amphibian cell. In a further aspect, a eukaryotic cell is an insect cell. In a further aspect, a eukaryotic cell is an arthropod cell. In a further aspect, a eukaryotic cell is a cephalopod cell. In a further aspect, a eukaryotic cell is an arachnid cell. In a further aspect, a eukaryotic cell is a mollusk cell. In a further aspect, a eukaryotic cell is a nematode cell. In a further aspect, a eukaryotic cell is a fish cell.

In another aspect, a eukaryotic cell is a protozoan cell. In another aspect, a eukaryotic cell is a fungal cell. In an aspect, a fungal cell is a yeast cell. In an aspect, a yeast cell is a Schizosaccharomyces pombe cell. In another aspect, a yeast cell is a Saccharomyces cerevisiae cell.

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

As used herein, the term “recombinant” in reference to a nucleic acid (DNA or RNA) molecule, protein, construct, vector, etc., refers to a nucleic acid or amino acid molecule or sequence that is man-made and not normally found in nature, and/or is present in a context in which it is not normally found in nature, including a nucleic acid molecule (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other.

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo.

In an aspect, this disclosure provides a plasmid comprising any nucleic acid molecule provided herein. In another aspect, this disclosure provides a plasmid encoding any amino acid sequence provided herein. In another aspect, this disclosure provides a plasmid encoding any ribonucleoprotein provided herein.

In another aspect, a nucleic acid encoding a Cas12a nuclease or CasX nuclease is provided in a vector. In a further aspect, a nucleic acid encoding a linker is provided in a vector. In a further aspect, a nucleic acid encoding a HUH endonuclease is provided in a vector. In a further aspect, a nucleic acid encoding a guide nucleic acid is provided in a vector. In a further aspect, a nucleic acid encoding a template nucleic acid molecule is provided in a vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et al., “Multiple sequence alignment with the Clustal series of programs,” Nucleic Acids Research 31:3497-3500 (2003); Thompson JD et al., “Clustal W: Improving the sensitivity of progressive multiple sequence alignment through sequence weighting, position-specific gap penalties and weight matrix choice,” Nucleic Acids Research 22:4673-4680 (1994); Larkin M A et al., “Clustal W and Clustal X version 2.0,” Bioinformatics 23:2947-48 (2007); and Altschul, S. F., Gish, W., Miller, W., Myers, E. W. & Lipman, D.J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine pairing with thymine, adenine pairing with uracil, and guanine (G) pairing with cytosine (C) [DNA, RNA]. In addition, it is also known in the art that for hybridization between two RNA molecules (e.g., dsRNA), guanine base pairs with uracil. For example, G/U base-pairing is partially responsible for the degeneracy (redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et al.). Typically, the length for a hybridizable nucleic acid is at least 10 nucleotides. Illustrative minimum lengths for a hybridizable nucleic acid are: at least 15 nucleotides; at least 18 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

As used herein, “in vivo” refers to within a living cell, tissue, or organism. As used herein, “in vitro” refers to within a labware. Non-limiting examples of labware include a test tube, a flask, a beaker, a graduated cylinder, a pipette, a petri dish, and a microtiter plate. As used herein, “ex vivo” refers to in a cell or tissue from an organism in an external environment. “Ex planta” refers to a plant cell or tissue in an external environment, while “in planta” refers to a cell within a living plant. As a non-limiting example, a plant protoplast in a petri dish or test tube would be considered both ex vivo and ex planta.

In an aspect, any nucleic acid molecule or polypeptide provided herein can be used in vivo. In an aspect, any nucleic acid molecule or polypeptide provided herein can be used in vitro. In an aspect, any nucleic acid molecule or polypeptide provided herein can be used ex vivo. In an aspect, any nucleic acid molecule or polypeptide provided herein can be used in planta. In an aspect, any nucleic acid molecule or polypeptide provided herein can be used ex planta.

In an aspect, any cell provided herein is present in vivo. In an aspect, any cell provided herein is present in vitro. In an aspect, any cell provided herein is present ex vivo. In an aspect, any plant cell provided herein is present in planta. In an aspect, any plant cell provided herein is present ex planta.

As commonly understood in the art, the term “promoter” refers to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. A promoter can be classified according to a variety of criteria relating to the pattern of expression of an associated coding or transcribable sequence or gene (including a transgene) operably linked to the promoter, such as constitutive, developmental, tissue-specific, inducible, etc.

In an aspect, a recombinant nucleic acid provided herein comprises at least one promoter. In another aspect, a polynucleotide encoding a Cas12a nuclease is operably linked to at least one promoter. In another aspect, a polynucleotide encoding a CasX nuclease is operably linked to at least one promoter. In another aspect, a polynucleotide encoding a HUH endonuclease is operably linked to at least one promoter. In another aspect, a polynucleotide encoding a FBNYV HUH endonuclease is operably linked to at least one promoter. In another aspect, a polynucleotide encoding a PCV HUH endonuclease is operably linked to at least one promoter. In another aspect, a polynucleotide encoding a guide nucleic acid is operably linked to at least one promoter. In another aspect, a polynucleotide encoding a template nucleic acid molecule is operably linked to at least one promoter.

Promoters that drive expression in all or most tissues of the plant are referred to as “constitutive” promoters. Promoters that drive expression during certain periods or stages of development are referred to as “developmental” promoters. Promoters that drive enhanced expression in certain tissues of an organism relative to other tissues of the organism are referred to as “tissue-preferred” promoters. Thus, a “tissue-preferred” promoter causes relatively higher or preferential expression in a specific tissue(s) of a plant, but with lower levels of expression in other tissue(s) of the plant. Promoters that express within a specific tissue(s) of an organism, with little or no expression in other tissues, are referred to as “tissue-specific” promoters. An “inducible” promoter is a promoter that initiates transcription in response to an environmental stimulus such as heat, cold, drought, light, or other stimuli, such as wounding or chemical application. A promoter can also be classified in terms of its origin, such as being heterologous, homologous, chimeric, synthetic, etc.

As used herein, the term “heterologous” in reference to a promoter is a promoter sequence having a different origin relative to its associated transcribable DNA sequence, coding sequence or gene (or transgene), and/or not naturally occurring in the plant species to be transformed. The term “heterologous” can refer more broadly to a combination of two or more DNA molecules or sequences, such as a promoter and an associated transcribable DNA sequence, coding sequence or gene, when such a combination is man-made and not normally found in nature.

In an aspect, a promoter provided herein is a constitutive promoter. In another aspect, a promoter provided herein is a tissue-specific promoter. In a further aspect, a promoter provided herein is a tissue-preferred promoter. In still another aspect, a promoter provided herein is an inducible promoter. In an aspect, a promoter provided herein is selected from the group consisting of a constitutive promoter, a tissue-specific promoter, a tissue-preferred promoter, and an inducible promoter.

RNA polymerase III (Pol III) promoters can be used to drive the expression of non-protein coding RNA molecules. In an aspect, a promoter provided herein is a Pol III promoter. In another aspect, a Pol III promoter provided herein is operably linked to a nucleic acid molecule encoding a non-protein coding RNA. In yet another aspect, a Pol III promoter provided herein is operably linked to a nucleic acid molecule encoding a guide nucleic acid. In still another aspect, a Pol III promoter provided herein is operably linked to a nucleic acid molecule encoding a single-guide RNA. In a further aspect, a Pol III promoter provided herein is operably linked to a nucleic acid molecule encoding a CRISPR RNA (crRNA). In another aspect, a Pol III promoter provided herein is operably linked to a nucleic acid molecule encoding a tracer RNA (tracrRNA).

Non-limiting examples of Pol III promoters include a U6 promoter, an H1 promoter, a 5S promoter, an Adenovirus 2 (Ad2) VAI promoter, a tRNA promoter, and a 7SK promoter. See, for example, Schramm and Hernandez, 2002, Genes & Development, 16:2593-2620, which is incorporated by reference herein in its entirety. In an aspect, a Pol III promoter provided herein is selected from the group consisting of a U6 promoter, an H1 promoter, a 5S promoter, an Adenovirus 2 (Ad2) VAI promoter, a tRNA promoter, and a 7SK promoter. In another aspect, a guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a single-guide RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a CRISPR RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter. In another aspect, a tracer RNA provided herein is operably linked to a promoter selected from the group consisting of a U6 promoter, an H1 promoter, a 5S promoter, an Adenovirus 2 (Ad2) VAI promoter, a tRNA promoter, and a 7SK promoter.

In an aspect, a promoter provided herein is a Dahlia Mosaic Virus (DaMV) promoter. In another aspect, a promoter provided herein is a U6 promoter. In another aspect, a promoter provided herein is an actin promoter.

Examples describing a promoter that can be used herein include without limitation U.S. Pat. No. 6,437,217 (maize RS81 promoter), U.S. Pat. No. 5,641,876 (rice actin promoter), U.S. Pat. No. 6,426,446 (maize RS324 promoter), U.S. Pat. No.

6,429,362 (maize PR-1 promoter), U.S. Pat. No. 6,232,526 (maize A3 promoter), U.S. Pat. No. 6,177,611 (constitutive maize promoters), U.S. Pat. Nos. 5,322,938, 5,352,605, 5,359,142 and 5,530,196 (35S promoter), U.S. Pat. No. 6,433,252 (maize L3 oleosin promoter), U.S. Pat. No. 6,429,357 (rice actin 2 promoter as well as a rice actin 2 intron), U.S. Pat. No. 5,837,848 (root specific promoter), U.S. Pat. No. 6,294,714 (light inducible promoters), U.S. Pat. No. 6,140,078 (salt inducible promoters), U.S. Pat. No. 6,252,138 (pathogen inducible promoters), U.S. Pat. No. 6,175,060 (phosphorus deficiency inducible promoters), U.S. Pat. No. 6,635,806 (gamma-coixin promoter), and U.S. patent application Ser. No. 09/757,089 (maize chloroplast aldolase promoter). Additional promoters that can find use are a nopaline synthase (NOS) promoter (Ebert et al., 1987), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Molecular Biology (1987) 9:315-324), the CaMV 35S promoter (Odell et al., Nature (1985) 313:810-812), the figwort mosaic virus 35S-promoter (U.S. Pat. Nos. 6,051,753; 5,378,619), the sucrose synthase promoter (Yang and Russell, Proceedings of the National Academy of Sciences, USA (1990) 87:4144-4148), the R gene complex promoter (Chandler et al., Plant Cell (1989) 1:1175-1183), and the chlorophyll a/b binding protein gene promoter, PC1SV (U.S. Pat. No. 5,850,019), and AGRtu.nos (GenBank Accession V00087; Depicker et al., Journal of Molecular and Applied Genetics (1982) 1:561-573; Bevan et al., 1983) promoters.

Promoter hybrids can also be used and constructed to enhance transcriptional activity (see U.S. Pat. No. 5,106,739), or to combine desired transcriptional activity, inducibility and tissue specificity or developmental specificity. Promoters that function in plants include but are not limited to promoters that are inducible, viral, synthetic, constitutive, temporally regulated, spatially regulated, and spatio-temporally regulated. Other promoters that are tissue-enhanced, tissue-specific, or developmentally regulated are also known in the art and envisioned to have utility in the practice of this disclosure.

It is appreciated in the art that a fragment of a promoter sequence can function to drive transcription of an operably linked nucleic acid molecule. For example, without being limiting, if a 1000 bp promoter is truncated to 500 bp, and the 500 bp fragment is capable of driving transcription, the 500 bp fragment is referred to as a “functional fragment.”

As used herein, a “nuclear localization signal” (NLS) refers to an amino acid sequence that “tags” a protein for import into the nucleus of a cell. In an aspect, a nucleic acid molecule provided herein encodes a nuclear localization signal. In another aspect, a nucleic acid molecule provided herein encodes two or more nuclear localization signals.

In an aspect, a Cas12a nuclease provided herein comprises a nuclear localization signal. In an aspect, a nuclear localization signal is positioned on the N-terminal end of a Cas12a nuclease. In a further aspect, a nuclear localization signal is positioned on the C-terminal end of a Cas12a nuclease. In yet another aspect, a nuclear localization signal is positioned on both the N-terminal end and the C-terminal end of a Cas12a nuclease.

In an aspect, a CasX nuclease provided herein comprises a nuclear localization signal. In an aspect, a nuclear localization signal is positioned on the N-terminal end of a CasX nuclease. In a further aspect, a nuclear localization signal is positioned on the C-terminal end of a CasX nuclease. In yet another aspect, a nuclear localization signal is positioned on both the N-terminal end and the C-terminal end of a CasX nuclease.

In an aspect, a HUH endonuclease provided herein comprises a nuclear localization signal. In an aspect, a nuclear localization signal is positioned on the N-terminal end of a HUH endonuclease. In a further aspect, a nuclear localization signal is positioned on the C-terminal end of a HUH endonuclease. In yet another aspect, a nuclear localization signal is positioned on both the N-terminal end and the C-terminal end of a HUH endonuclease.

In an aspect, a FBNYV HUH endonuclease provided herein comprises a nuclear localization signal. In an aspect, a nuclear localization signal is positioned on the N-terminal end of a FBNYV HUH endonuclease. In a further aspect, a nuclear localization signal is positioned on the C-terminal end of a FBNYV HUH endonuclease. In yet another aspect, a nuclear localization signal is positioned on both the N-terminal end and the C-terminal end of a FBNYV HUH endonuclease.

In an aspect, a PCV HUH endonuclease provided herein comprises a nuclear localization signal. In an aspect, a nuclear localization signal is positioned on the N-terminal end of a PCV HUH endonuclease. In a further aspect, a nuclear localization signal is positioned on the C-terminal end of a PCV HUH endonuclease. In yet another aspect, a nuclear localization signal is positioned on both the N-terminal end and the C-terminal end of a PCV HUH endonuclease.

In an aspect, a ribonucleoprotein comprises at least one nuclear localization signal. In another aspect, a ribonucleoprotein comprises at least two nuclear localization signals.

In an aspect, a nuclear localization signal provided herein is encoded by SEQ ID NO: 8. In an aspect, a nuclear localization signal provided herein is encoded by SEQ ID NO: 9. In another aspect, a nuclear localization signal is selected from the group consisting of SEQ ID NOs: 8 and 9.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www[dot]kazusa[dot]or[dot]jp[forwards slash]codon and these tables can be adapted in a number of ways. See Nakamura et al., 2000, Nucl. Acids Res. 28:292. Computer algorithms for codon optimizing a particular sequence for expression in a particular host cell are also available, such as Gene Forge (Aptagen; Jacobus, PA), are also available.

As used herein, “codon optimization” refers to a process of modifying a nucleic acid sequence for enhanced expression in a host cell of interest by replacing at least one codon (e.g., at least 1, 2, 3, 4, 5, 10, 15, 20, 25, 50, or more codons) of a sequence with codons that are more frequently or most frequently used in the genes of the host cell while maintaining the original amino acid sequence (e.g., introducing silent mutations).

In an aspect, one or more codons (e.g., 1, 2, 3, 4, 5, 10, 15, 20, 25, 50, or more, or all codons) in a sequence encoding a Cas12a nuclease, a CasX nuclease, or a HUH endonuclease correspond to the most frequently used codon for a particular amino acid. As to codon usage in plants, including algae, reference is made to Campbell and Gowri, 1990, Plant Physiol., 92:1-11; and Murray et al., 1989, Nucleic Acids Res., 17:477-98, each of which is incorporated herein by reference in their entireties.

In one aspect, a nucleic acid molecule provided herein encodes a Cas12a nuclease that is codon optimized for a prokaryotic cell. In one aspect, a nucleic acid molecule provided herein encodes a Cas12a nuclease that is codon optimized for a Escherichia coli cell. In one aspect, a nucleic acid molecule provided herein encodes a Cas12a nuclease that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a Cas12a nuclease that is codon optimized for a plant cell. In another aspect, a nucleic acid molecule provided herein encodes a Cas12a nuclease that is codon optimized for a monocotyledonous plant species. In another aspect, a protein-coding nucleic acid molecule is codon optimized for a dicotyledonous plant species. In a further aspect, a nucleic acid molecule provided herein encodes a Cas12a nuclease that is codon optimized for a gymnosperm plant species. In a further aspect, a nucleic acid molecule provided herein encodes a Cas12a nuclease that is codon optimized for an angiosperm plant species. In a further aspect, a nucleic acid molecule provided herein encodes a Cas12a nuclease that is codon optimized for a corn cell. In a further aspect, a nucleic acid molecule provided herein encodes a Cas 12a nuclease that is codon optimized for a soybean cell. In a further aspect, a nucleic acid molecule provided herein encodes a Cas12a nuclease that is codon optimized for a rice cell. In a further aspect, a nucleic acid molecule provided herein encodes a Cas12a nuclease that is codon optimized for a wheat cell. In a further aspect, a nucleic acid molecule provided herein encodes a Cas12a nuclease that is codon optimized for a cotton cell. In a further aspect, a nucleic acid molecule provided herein encodes a Cas12a nuclease that is codon optimized for a sorghum cell. In a further aspect, a nucleic acid molecule provided herein encodes a Cas12a nuclease that is codon optimized for an alfalfa cell. In a further aspect, a nucleic acid molecule provided herein encodes a Cas12a nuclease that is codon optimized for a sugar cane cell. In a further aspect, a nucleic acid molecule provided herein encodes a Cas12a nuclease that is codon optimized for an Arabidopsis cell. In a further aspect, a nucleic acid molecule provided herein encodes a Cas12a nuclease that is codon optimized for a tomato cell. In a further aspect, a nucleic acid molecule provided herein encodes a Cas12a nuclease that is codon optimized for a cucumber cell. In a further aspect, a nucleic acid molecule provided herein encodes a Cas12a nuclease that is codon optimized for a potato cell. In a further aspect, a nucleic acid molecule provided herein encodes a Cas12a nuclease that is codon optimized for an algae cell.

In one aspect, a nucleic acid molecule provided herein encodes a CasX nuclease that is codon optimized for a prokaryotic cell. In one aspect, a nucleic acid molecule provided herein encodes a CasX nuclease that is codon optimized for a Escherichia coli cell. In one aspect, a nucleic acid molecule provided herein encodes a CasX nuclease that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a CasX nuclease that is codon optimized for a plant cell. In another aspect, a nucleic acid molecule provided herein encodes a CasX nuclease that is codon optimized for a monocotyledonous plant species. In another aspect, a protein-coding nucleic acid molecule is codon optimized for a dicotyledonous plant species. In a further aspect, a nucleic acid molecule provided herein encodes a CasX nuclease that is codon optimized for a gymnosperm plant species. In a further aspect, a nucleic acid molecule provided herein encodes a CasX nuclease that is codon optimized for an angiosperm plant species. In a further aspect, a nucleic acid molecule provided herein encodes a CasX nuclease that is codon optimized for a corn cell. In a further aspect, a nucleic acid molecule provided herein encodes a CasX nuclease that is codon optimized for a soybean cell. In a further aspect, a nucleic acid molecule provided herein encodes a CasX nuclease that is codon optimized for a rice cell. In a further aspect, a nucleic acid molecule provided herein encodes a CasX nuclease that is codon optimized for a wheat cell. In a further aspect, a nucleic acid molecule provided herein encodes a CasX nuclease that is codon optimized for a cotton cell. In a further aspect, a nucleic acid molecule provided herein encodes a CasX nuclease that is codon optimized for a sorghum cell. In a further aspect, a nucleic acid molecule provided herein encodes a CasX nuclease that is codon optimized for an alfalfa cell. In a further aspect, a nucleic acid molecule provided herein encodes a CasX nuclease that is codon optimized for a sugar cane cell. In a further aspect, a nucleic acid molecule provided herein encodes a CasX nuclease that is codon optimized for an Arabidopsis cell. In a further aspect, a nucleic acid molecule provided herein encodes a CasX nuclease that is codon optimized for a tomato cell. In a further aspect, a nucleic acid molecule provided herein encodes a CasX nuclease that is codon optimized for a cucumber cell. In a further aspect, a nucleic acid molecule provided herein encodes a CasX nuclease that is codon optimized for a potato cell. In a further aspect, a nucleic acid molecule provided herein encodes a CasX nuclease that is codon optimized for an algae cell.

In one aspect, a nucleic acid molecule provided herein encodes a HUH endonuclease that is codon optimized for a prokaryotic cell. In one aspect, a nucleic acid molecule provided herein encodes a HUH endonuclease that is codon optimized for a Escherichia coli cell. In one aspect, a nucleic acid molecule provided herein encodes a HUH endonuclease that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a HUH endonuclease that is codon optimized for a plant cell. In another aspect, a nucleic acid molecule provided herein encodes a HUH endonuclease that is codon optimized for a monocotyledonous plant species. In another aspect, a protein-coding nucleic acid molecule is codon optimized for a dicotyledonous plant species. In a further aspect, a nucleic acid molecule provided herein encodes a HUH endonuclease that is codon optimized for a gymnosperm plant species. In a further aspect, a nucleic acid molecule provided herein encodes a HUH endonuclease that is codon optimized for an angiosperm plant species. In a further aspect, a nucleic acid molecule provided herein encodes a HUH endonuclease that is codon optimized for a corn cell. In a further aspect, a nucleic acid molecule provided herein encodes a HUH endonuclease that is codon optimized for a soybean cell. In a further aspect, a nucleic acid molecule provided herein encodes a HUH endonuclease that is codon optimized for a rice cell. In a further aspect, a nucleic acid molecule provided herein encodes a HUH endonuclease that is codon optimized for a wheat cell. In a further aspect, a nucleic acid molecule provided herein encodes a HUH endonuclease that is codon optimized for a cotton cell. In a further aspect, a nucleic acid molecule provided herein encodes a HUH endonuclease that is codon optimized for a sorghum cell. In a further aspect, a nucleic acid molecule provided herein encodes a HUH endonuclease that is codon optimized for an alfalfa cell. In a further aspect, a nucleic acid molecule provided herein encodes a HUH endonuclease that is codon optimized for a sugar cane cell. In a further aspect, a nucleic acid molecule provided herein encodes a HUH endonuclease that is codon optimized for an Arabidopsis cell. In a further aspect, a nucleic acid molecule provided herein encodes a HUH endonuclease that is codon optimized for a tomato cell. In a further aspect, a nucleic acid molecule provided herein encodes a HUH endonuclease that is codon optimized for a cucumber cell. In a further aspect, a nucleic acid molecule provided herein encodes a HUH endonuclease that is codon optimized for a potato cell. In a further aspect, a nucleic acid molecule provided herein encodes a HUH endonuclease that is codon optimized for an algae cell.

In one aspect, a nucleic acid molecule provided herein encodes a FBNYV HUH endonuclease that is codon optimized for a prokaryotic cell. In one aspect, a nucleic acid molecule provided herein encodes a FBNYV HUH endonuclease that is codon optimized for a Escherichia coli cell. In one aspect, a nucleic acid molecule provided herein encodes a FBNYV HUH endonuclease that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a FBNYV HUH endonuclease that is codon optimized for a plant cell. In another aspect, a nucleic acid molecule provided herein encodes a FBNYV HUH endonuclease that is codon optimized for a monocotyledonous plant species. In another aspect, a protein-coding nucleic acid molecule is codon optimized for a dicotyledonous plant species. In a further aspect, a nucleic acid molecule provided herein encodes a FBNYV HUH endonuclease that is codon optimized for a gymnosperm plant species. In a further aspect, a nucleic acid molecule provided herein encodes a FBNYV HUH endonuclease that is codon optimized for an angiosperm plant species. In a further aspect, a nucleic acid molecule provided herein encodes a FBNYV HUH endonuclease that is codon optimized for a corn cell. In a further aspect, a nucleic acid molecule provided herein encodes a FBNYV HUH endonuclease that is codon optimized for a soybean cell. In a further aspect, a nucleic acid molecule provided herein encodes a FBNYV HUH endonuclease that is codon optimized for a rice cell. In a further aspect, a nucleic acid molecule provided herein encodes a FBNYV HUH endonuclease that is codon optimized for a wheat cell. In a further aspect, a nucleic acid molecule provided herein encodes a FBNYV HUH endonuclease that is codon optimized for a cotton cell. In a further aspect, a nucleic acid molecule provided herein encodes a FBNYV HUH endonuclease that is codon optimized for a sorghum cell. In a further aspect, a nucleic acid molecule provided herein encodes a FBNYV HUH endonuclease that is codon optimized for an alfalfa cell. In a further aspect, a nucleic acid molecule provided herein encodes a FBNYV HUH endonuclease that is codon optimized for a sugar cane cell. In a further aspect, a nucleic acid molecule provided herein encodes a FBNYV HUH endonuclease that is codon optimized for an Arabidopsis cell. In a further aspect, a nucleic acid molecule provided herein encodes a FBNYV HUH endonuclease that is codon optimized for a tomato cell. In a further aspect, a nucleic acid molecule provided herein encodes a FBNYV HUH endonuclease that is codon optimized for a cucumber cell. In a further aspect, a nucleic acid molecule provided herein encodes a FBNYV HUH endonuclease that is codon optimized for a potato cell. In a further aspect, a nucleic acid molecule provided herein encodes a FBNYV HUH endonuclease that is codon optimized for an algae cell.

In one aspect, a nucleic acid molecule provided herein encodes a PCV HUH endonuclease that is codon optimized for a prokaryotic cell. In one aspect, a nucleic acid molecule provided herein encodes a PCV HUH endonuclease that is codon optimized for a Escherichia coli cell. In one aspect, a nucleic acid molecule provided herein encodes a PCV HUH endonuclease that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a PCV HUH endonuclease that is codon optimized for a plant cell. In another aspect, a nucleic acid molecule provided herein encodes a PCV HUH endonuclease that is codon optimized for a monocotyledonous plant species. In another aspect, a protein-coding nucleic acid molecule is codon optimized for a dicotyledonous plant species. In a further aspect, a nucleic acid molecule provided herein encodes a PCV HUH endonuclease that is codon optimized for a gymnosperm plant species. In a further aspect, a nucleic acid molecule provided herein encodes a PCV HUH endonuclease that is codon optimized for an angiosperm plant species. In a further aspect, a nucleic acid molecule provided herein encodes a PCV HUH endonuclease that is codon optimized for a corn cell. In a further aspect, a nucleic acid molecule provided herein encodes a PCV HUH endonuclease that is codon optimized for a soybean cell. In a further aspect, a nucleic acid molecule provided herein encodes a PCV HUH endonuclease that is codon optimized for a rice cell. In a further aspect, a nucleic acid molecule provided herein encodes a PCV HUH endonuclease that is codon optimized for a wheat cell. In a further aspect, a nucleic acid molecule provided herein encodes a PCV HUH endonuclease that is codon optimized for a cotton cell. In a further aspect, a nucleic acid molecule provided herein encodes a PCV HUH endonuclease that is codon optimized for a sorghum cell. In a further aspect, a nucleic acid molecule provided herein encodes a PCV HUH endonuclease that is codon optimized for an alfalfa cell. In a further aspect, a nucleic acid molecule provided herein encodes a PCV HUH endonuclease that is codon optimized for a sugar cane cell. In a further aspect, a nucleic acid molecule provided herein encodes a PCV HUH endonuclease that is codon optimized for an Arabidopsis cell. In a further aspect, a nucleic acid molecule provided herein encodes a PCV HUH endonuclease that is codon optimized for a tomato cell. In a further aspect, a nucleic acid molecule provided herein encodes a PCV HUH endonuclease that is codon optimized for a cucumber cell. In a further aspect, a nucleic acid molecule provided herein encodes a PCV HUH endonuclease that is codon optimized for a potato cell. In a further aspect, a nucleic acid molecule provided herein encodes a PCV HUH endonuclease that is codon optimized for an algae cell.

In one aspect, a nucleic acid molecule provided herein encodes a linker that is codon optimized for a prokaryotic cell. In one aspect, a nucleic acid molecule provided herein encodes a linker that is codon optimized for a Escherichia coli cell. In one aspect, a nucleic acid molecule provided herein encodes a linker that is codon optimized for a eukaryotic cell. In an aspect, a nucleic acid molecule provided herein is codon optimized for an animal cell. In an aspect, a nucleic acid molecule provided herein is codon optimized for a fungus cell. In an aspect, a nucleic acid molecule provided herein is codon optimized for a yeast cell. In another aspect, a nucleic acid molecule provided herein encodes a linker that is codon optimized for a plant cell. In another aspect, a nucleic acid molecule provided herein encodes a linker that is codon optimized for a monocotyledonous plant species. In another aspect, a protein-coding nucleic acid molecule is codon optimized for a dicotyledonous plant species. In a further aspect, a nucleic acid molecule provided herein encodes a linker that is codon optimized for a gymnosperm plant species. In a further aspect, a nucleic acid molecule provided herein encodes a linker that is codon optimized for an angiosperm plant species. In a further aspect, a nucleic acid molecule provided herein encodes a linker that is codon optimized for a corn cell. In a further aspect, a nucleic acid molecule provided herein encodes a linker that is codon optimized for a soybean cell. In a further aspect, a nucleic acid molecule provided herein encodes a linker that is codon optimized for a rice cell. In a further aspect, a nucleic acid molecule provided herein encodes a linker that is codon optimized for a wheat cell. In a further aspect, a nucleic acid molecule provided herein encodes a linker that is codon optimized for a cotton cell. In a further aspect, a nucleic acid molecule provided herein encodes a linker that is codon optimized for a sorghum cell. In a further aspect, a nucleic acid molecule provided herein encodes a linker that is codon optimized for an alfalfa cell. In a further aspect, a nucleic acid molecule provided herein encodes a linker that is codon optimized for a sugar cane cell. In a further aspect, a nucleic acid molecule provided herein encodes a linker that is codon optimized for an Arabidopsis cell. In a further aspect, a nucleic acid molecule provided herein encodes a linker that is codon optimized for a tomato cell. In a further aspect, a nucleic acid molecule provided herein encodes a linker that is codon optimized for a cucumber cell. In a further aspect, a nucleic acid molecule provided herein encodes a linker that is codon optimized for a potato cell. In a further aspect, a nucleic acid molecule provided herein encodes a linker that is codon optimized for an algae cell.

### EXAMPLES

### Example 1

### Design of Expression Vectors

Cys-free LbCas 12a protein (SEQ ID NO: 1) was fused to an HUH endonuclease (SEQ ID NO: 2) from Faba Bean Necrotic Yellow Virus (FBNYV) via a novel sixteen amino-acid-long flexible linker (SEQ ID NO: 3). One of the optimal features of the linker is the presence of a high number of glycine, serine, and threonine residues. Without being limited by any scientific theory, these residues are not hydrophobic and can readily associate with the solvent in between the two protein domains. Without being limited by any scientific theory, these residues are also among the most flexible amino acids thereby providing the linker the ability to adopt many different conformations which in turn allows the two protein domains the freedom to move relative to one another. Also without being limited by any scientific theory, the aspartic acid and glutamic acid residues are highly charged, a feature that promotes interaction with the solvent, reducing the likelihood of protein aggregation.

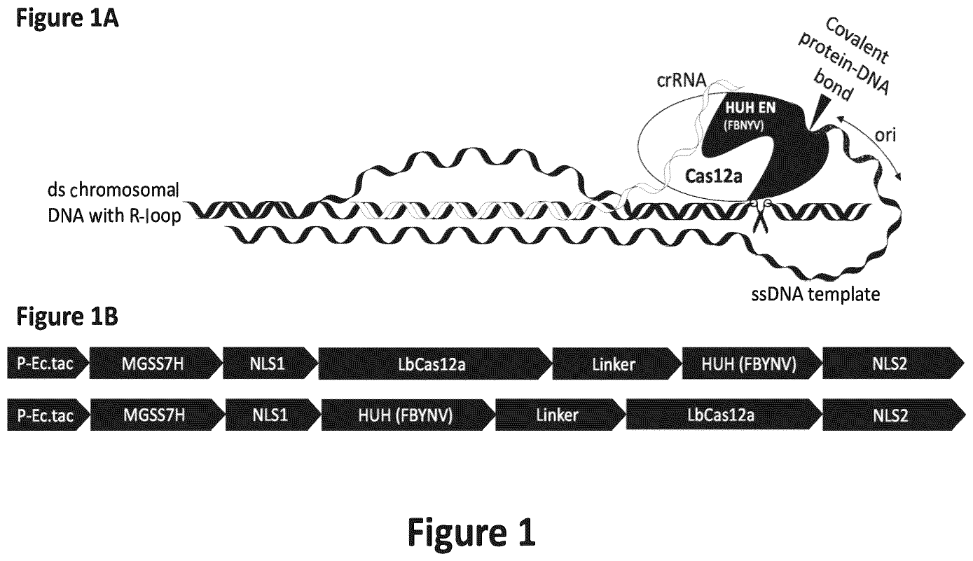

The sequences encoding the three components of the fusion protein (cys-free LbCas12a, linker, HUH) were codon-optimized for optimal expression in Escherichia coli. The nucleotide sequence encoding cys-free LbCas12a is set forth as SEQ ID NO: 4. The nucleotide sequence encoding HUH endonuclease is set forth as SEQ ID NO: 5, and the nucleotide sequence encoding the linker is set forth as SEQ ID NO:6. Both N- and C-terminal HUH fusion proteins were designed. The nucleotide sequence encoding the cys-free LbCas12a:linker:HUH is set forth as SEQ ID NO: 7 and the nucleotide sequence encoding the HUH:linker:cys-free LbCas12a is set forth as SEQ ID NO: 17. Nuclear localization signals (NLS) (SEQ ID NOs: 8 and 9) were introduced at the 5′- and 3′-ends of the LbCas12a-linker:HUH open reading frame, respectively. Additionally, a nucleotide sequence encoding a histidine (HIS) tag (SEQ ID NO:10) was introduced 5′ to the 5′ NLS sequence. The nucleotide sequence encoding the HIS tag: NLS1:cys-free LbCas12a:linker:HUH:NLS2 (referred to below as “cys-free LbCas12a:HUH”) sequence was placed under the control of the P-Ec.tac promoter (SEQ ID NO:11) and inserted into a bacterial expression vector. Graphical illustrations of the N- and C-terminal HUH fusion protein expression cassettes are shown in FIG. 1B. A control expression vector comprising a HIS tag:NLS1:cys-freeLbCas12a:NLS2 (referred to below as “cys-free LbCas12a control”) sequence under the control of the P-Ec.tac promoter was also designed. See FIG. 1B. cys-free LbCas12a:HUH and cys-free LbCas12a control proteins were expressed and purified from E. coli cells transformed with the expression vectors described above.

### Example 2

### Testing of Fusion Proteins

To test whether cys-free LbCas12a:HUH could recognize and cleave chromosomal DNA in the presence of a cognate guide RNA, three unique target sites within the soybean (Glycine max) genome were chosen. crRNAs were designed to guide the cys-free LbCas12a:HUH and cys-free LbCas12a control proteins to each target site. Ribonucleoprotein (RNP) complexes comprising the purified cys-free LbCas12a:HUH fusion protein or cys-free LbCas12a and cognate crRNAs were assembled.

A 70-nucleotide long ssDNA template was designed. The template comprises a 10-nucleotide signature motif flanked by homology arms that comprise homology to sequences flanking the GmTS1 site. The ssDNA template was fused to the 15-nucleotide origin (ori) HUH recognition sequence (SEQ ID NO:12) of PCV. As shown in Example 6, the ori sequence from PCV is compatible with the FBNYV HUH endonuclease.

This single-stranded DNA template (ssDNA template) was added to the RNP complex. Next, this tri-partite RNP complex (see FIG. 1A) was tested in vitro for the expected functionalities of the two fusion domains: (a) targeted DNA cleavage and (b) covalent tethering of the ssDNA template. A gel-shift assay was performed to investigate the covalent tethering of the ssDNA template to cys-free LbCas12a:HUH (see Table 1). The gel-shift assay confirmed that in the presence of cys-free LbCas12a:HUH, the ssDNA template migrated significantly slower than it did in the either the presence of either cys-free LbCas12a control or the ssDNA template in its unbound state (see Table 1). The slower migration is indicative of the formation of cys-free LbCas12a:HUH::ssDNA tethered complexes.

TABLE 1
Gel shift assay investigating the covalent tethering
of the ssDNA template to cys-free LbCas12a:HUH.

| | | | | | Upward |
|---|---|---|---|---|---|
| | | | | | shift |
| | | cys-free | | | in ssDNA |
| Assay | cys-free | LbCas12a | | ssDNA | migration |
| type | LbCas12a:HUH | Control | crRNA | template | observed |
| Control | − | − | − | + | No |
| Test | + | − | crRNA- | + | Yes |
| | | | TS1 | | |
| Control | − | + | crRNA- | + | No |
| | | | TS1 | | |
| Control | − | + | crRNA- | − | N/A |
| | | | TS1 | | |
| Control | − | − | − | + | No |
| Test | + | − | crRNA- | + | Yes |
| | | | TS2 | | |
| Control | − | + | crRNA- | + | No |
| | | | TS2 | | |
| Control | − | + | crRNA- | − | N/A |
| | | | TS2 | | |
| Control | − | − | − | + | No |
| Test | + | − | crRNA- | + | Yes |
| | | | TS3 | | |
| Control | − | + | crRNA- | + | No |
| | | | TS3 | | |
| Control | − | + | crRNA- | − | N/A |
| | | | TS3 | | |

“+” indicates presence;
“−” indicates absence.
“TS1” revers to GmTS1;
“TS2” refers to GmTS2; and
“TS3” refers to GmTS3.
“N/A” refers to Not applicable.

The ability of cys-free LbCas12a:HUH to cleave target DNA was assessed via an in vitro assay. A 1020-nucleotide PCR amplicon spanning two of the target sites (GmTS1 and GmTS2) was generated from the wild type soybean germplasm A3555. The ability of cys-free LbCas12a:HUH to cleave the PCR amplicon at expected sites in the presence of the cognate crRNAs was tested. Cys-freeLbCas12a was used as a positive control and assays lacking nucleases served as negative controls.

Targeted-cleavage of PCR amplicon by cys-freeLbCas12a in the presence of crRNA-TS1 is expected to result in 892-nucleotide and 128-nucleotide digestion products.

Targeted cleavage of PCR amplicon by cys-free LbCas12a in the presence of crRNA-TS2 is expected to result in 734-nucleotide and 286-nucleotide digestion products. As shown in Table 2, the PCR amplicon was digested by both the cys-free LbCas12a:HUH complexes and cys-free LbCas 12a control complexes in the expected patterns.

TABLE 2
Investigating the cleavage capabilities of cys-free LbCas12a:HUH
and cys-free LbCas12a control fusion proteins.

| | | | | | Digestion |
|---|---|---|---|---|---|
| Type of | cys-free | cys-free | | | products |
| assay | LbCas12a:HUH | LbCas12a | crRNA | ssDNA | observed |
| Test | + | − | crRNA- | + | 892 nt; |
| | | | TS1 | | 128 nt |
| Positive | − | + | crRNA- | + | 892 nt; |
| Control | | | TS1 | | 128 nt |
| Negative | − | − | crRNA- | + | none |
| Control | | | TS1 | | |
| Test | + | − | crRNA- | + | 734 nt; |
| | | | TS2 | | 286 nt |
| Positive | − | + | crRNA- | + | 734 nt; |
| Control | | | TS2 | | 286 nt |
| Negative | − | − | crRNA- | + | none |
| Control | | | TS2 | | |

“+” indicates presence;
“−” indicates absence.
“TS1” refers to GmTS1;
“TS2” refers to GmTS2.

### Example 3

### Testing of Fusion Proteins in Protoplasts

The tripartite cys-free LbCas12a:HUH/crRNA/ssDNA template RNP complex was tested for chromosome cleavage in protoplast cells. Protoplasts were prepared from soybean embryos and were subsequently transformed with the RNP complexes comprising cys-free LbCas12a:HUH/crRNA/ssDNA template and their positive control counterparts carrying cys-free LbCas12a, by standard methods known in the art. Negative controls included matching crRNAs and ssDNA templates, but no fusion proteins (see Table 3).

A qualitative assay (Table 3) was developed to investigate the activity of cys-free LbCas12a:HUH. Following a two-day incubation at room temperature, total genomic DNA was isolated from all samples and 1020 nucleotide PCR amplicons spanning target sites GmTS1 and GmTS2 were generated. The amplicons were expected to comprise a heterogenous population of DNA sequences that comprised wildtype and edited target sites, the latter comprising INDELs. The PCR amplified amplicons were subsequently re-digested by cys-free LbCas 12a in vitro. Undigested amplicons, indicative of edited target sites, were observed in both test and positive controls. Undigested amplicons were undetectable in negative controls. Sequencing of the undigested fractions confirmed presence of targeted INDELS in both tests and positive controls.

TABLE 3
Assay investigating the activity of cys-freeLbCas12-HUH in protoplast cells.

| | | | | | | Undigested |
|---|---|---|---|---|---|---|
| Type of | PCR | cys-free | cys-free | crRNA- | | Amplicons |
| Assay | Amplicon | LbCas12a:HUH | LbCas12a | TS2 | Template | observed |
| Test | + | + | − | + | + | Yes |
| Positive | + | − | + | + | + | Yes |
| control | | | | | | |
| Negative | + | − | − | + | + | No |
| control | | | | | | |

“+” indicates presence; “−” indicates absence.

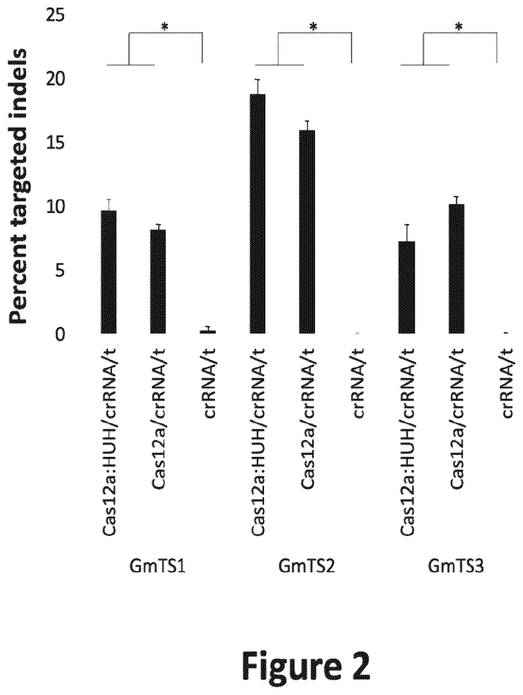

For quantitative comparison among treatments, amplicons spanning the target site were generated and sequenced by Next Generation Sequencing (NGS), by standard methods known in the art. Sequencing reads were considered mutant if they included indels in the seven-nucleotide LbCas12a cut site, which was positioned 18 to 24 nucleotides downstream of the LbCas12a PAM site. Seven to nineteen percent indel rates were detected in both test and positive controls across all three target sites tested. The negative controls had no more than 0.3% indel rates in the same region, which is significantly below the test/positive control cleavage rates for all sites (FIG. 2).

### Example 4

### In Planta Testing of Fusion Proteins

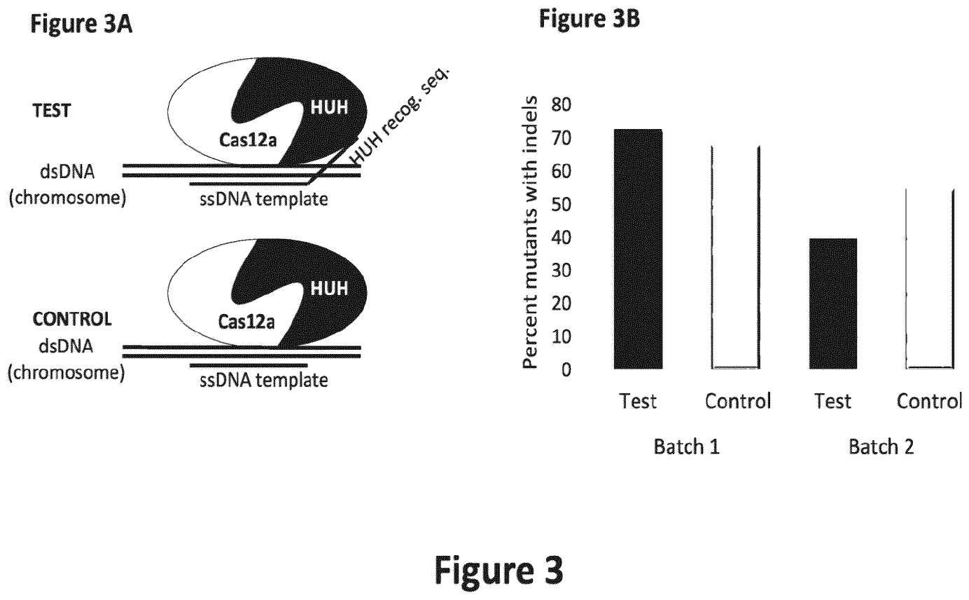

The cys-freeLbCas12a:HUH/crRNA/template complex was tested for chromosome cleavage and for targeted integrations in planta. The test and control samples both included the cys-freeLbCas12a:HUH fusion proteins and the crRNA for the GmTS1 target site as described in Example 1. The template in the Test assay included the ssDNA template comprising a 15-nucleotide ori HUH-recognition sequence from PCV as described in Example 2. The control assay comprised an ssDNA template that lacked the ori sequence. See FIG. 3A.

RNP complexes were assembled to be used for plant transformation. Two independent transformations were performed using particle bombardment. The RNP (cys-free LbCas12a:HUH/crRNA/template) complex together with a linear dsDNA fragment encoding for the spectinomycin resistance gene aadA was coated on 0.6 μm gold particles using a mixture of CaCl₂ and spermidine as coating agents. The coated particles were delivered into soybean dry excised embryos using the Biolistic PDS-1000/HE Particle Delivery System (Biorad).

Transformed seedlings were grown in tissue culture using spectinomycin for selection. Assuming near-stochiometric delivery of RNPs and the spectinomycin selectable marker gene, surviving plants were expected to be enriched for those carrying the RNP complex. Seedlings with at least one trifoliate leaf were sampled for DNA analysis.

Amplicons spanning the GmTS1 target site were generated and subjected to NGS using standard methods known in the art. Chromosome cleavage activity of cys-free LbCas12a:HUH was determined by quantifying the presence of targeted indels within the 7-nucleotide GmTS1cut site. Plants that generated at least 20% targeted indel reads were called mutants (see Table 4; and FIG. 3B). Mutation rates were >30% in all treatments, suggesting robust cleavage activity by the cys-freeLbCas12a:HUH fusion protein. (see Table 4; and FIG. 3B).

TABLE 4
Results of in planta editing using cys-free
LbCas12a:HUH/crRNA/template with an ori sequence
(Test) and cys-free LbCas12a:HUH/crRNA/template
without an ori sequence (Control).

| | Batch 1 | | | Batch 2 | | |
|---|---|---|---|---|---|---|
| | Test | Control | P Value | Test | Control | P Value |
| Total R0 Events | 239 | 153 | | 113 | 182 | |
| Total mutants | 173 | 104 | | 45 | 100 | |
| (20% mutant read | | | | | | |
| threshold) | | | | | | |
| Percent mutants | 72.4 | 68.0 | 0.35 | 39.8 | 54.9 | 0.010 |
| (20% mutant read | | | | | | |
| threshold) | | | | | | |

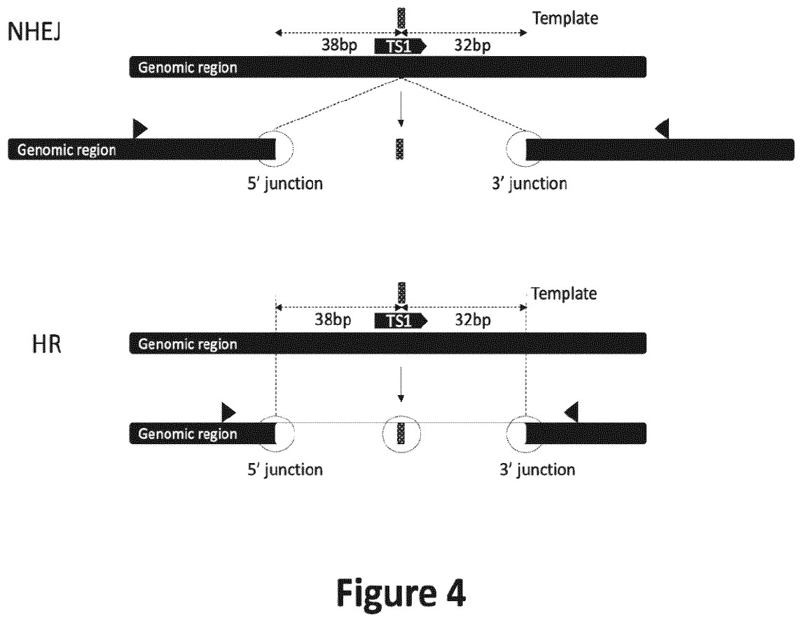

Targeted integrations were defined by the presence of a unique signature motif of the template in the NGS amplicons spanning the GmTS1 target site (see FIG. 4). Integrations can occur through two basic DNA repair processes, non-homologous end-joining (NHEJ) and homologous recombination (HR), or a combination of NHEJ and HR. Different template-chromosomes junctions are created by NHEJ and HR, which can be used to determine the mechanism of integration (see FIG. 4).

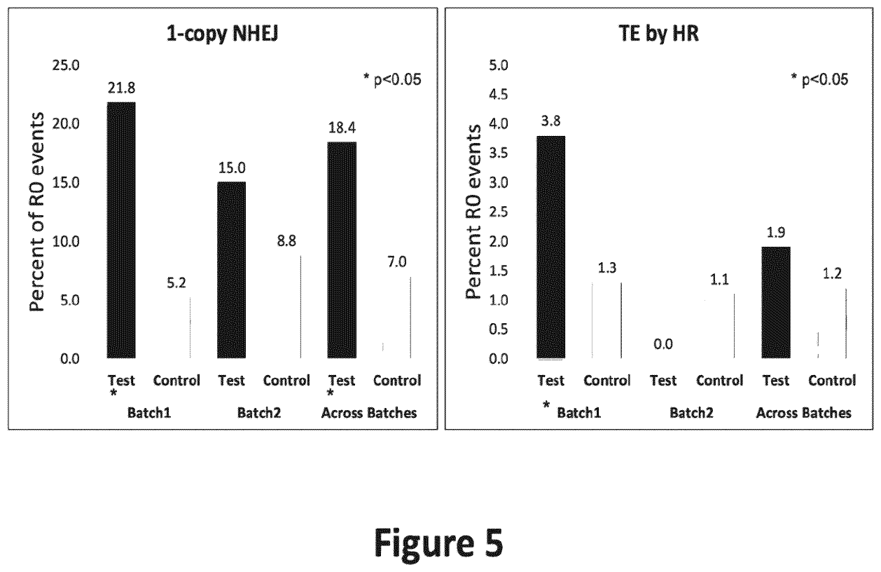

Overall, HUH-mediated tethering of exogenous ssDNA to cys-free LbCas12a promoted targeted integrations (See Table 5; Table 6; and FIG. 5). For example, integration of single-copy template by NHEJ was significantly higher in tests as compared to controls in all batches. For homologous recombination, up to 3.8% integration was detected in tests as compared to no more than 1.3% in controls. Covalently tethering a ssDNA template to LbCas12a:HUH enhances targeted template integration in planta.

TABLE 5
In planta testing of LbCas12a:HUH fusion proteins for targeted
integrations using cys-freeLbCas12a:HUH/crRNA/template
with an ori sequence (Test) and
cys-freeLbCas12a:HUH/crRNA/template
without an ori sequence (Control).

| | Batch 1 | | Batch 2 | |
|---|---|---|---|---|
| | Test | Control | Test | Control |
| Total R0 Events | 239 | 153 | 113 | 182 |
| 1-copy NHEJ | 52 | 8 | 17 | 16 |
| 2-copy NHEJ | 3 | 0 | 2 | 2 |
| Total NHEJ | 55 | 8 | 19 | 18 |
| TE by HR | 9 | 2 | 0 | 2 |
| Total integration | 64 | 10 | 19 | 20 |
| (NHEJ + HR) | | | | |

“TE” refers to templated editing via integration of template DNA by HR.
“1-copy” indicates single copy of the template inserted within the target site.
“2-copy” indicates two copies of the template integrated into the target site

TABLE 6
In planta testing of LbCas12a:HUH fusion proteins for targeted integrations
using cys-free LbCas12a:HUH/crRNA/template with an ori sequence
(Test) and cys-free LbCas12a:HUH/crRNA/template without an ori
sequence (Control). Percentages are based on the total number of
plants described above in Table 5. “TE” refers to templated
editing via integration of template DNA by HR.

| | | | | | | | p-value |
|---|---|---|---|---|---|---|---|
| | Batch 1 | | | Batch 2 | | | across |
| | Test | Control | p-value | Test | Control | p-value | batches |
| Total R0 | 100% | 100% | N/A | 100% | 100% | N/A | N/A |
| Events | | | | | | | |
| 1-copy NHEJ | 21.8% | 5.20% | 2.8 × 10⁻⁷* | 15.0% | 8.8% | 0.11 | 0.023* |
| 2-copy NHEJ | 1.3% | 0% | 0.16 | 1.8% | 1.1% | 0.64 | 0.15 |
| Total NHEJ | 23.1% | 5.20% | 5.07 × 10⁻⁸* | 16.8% | 9.9% | 0.095 | 0.012* |
| TE by HR | 3.80% | 1.30% | 0.11 | 0.0% | 1.1% | 0.29 | 0.77 |
| Total | 26.9% | 6.5% | 6.79 × 10⁻⁹* | 16.8% | 11.0% | 0.17 | 0.066 |
| integration | | | | | | | |
| (NHEJ + HR) | | | | | | | |

*indicates a statistically significant p-value.
N/A refers to not applicable.

### Example 5

### Testing of N- and C-Terminal HUH Fusion Proteins in Protoplasts

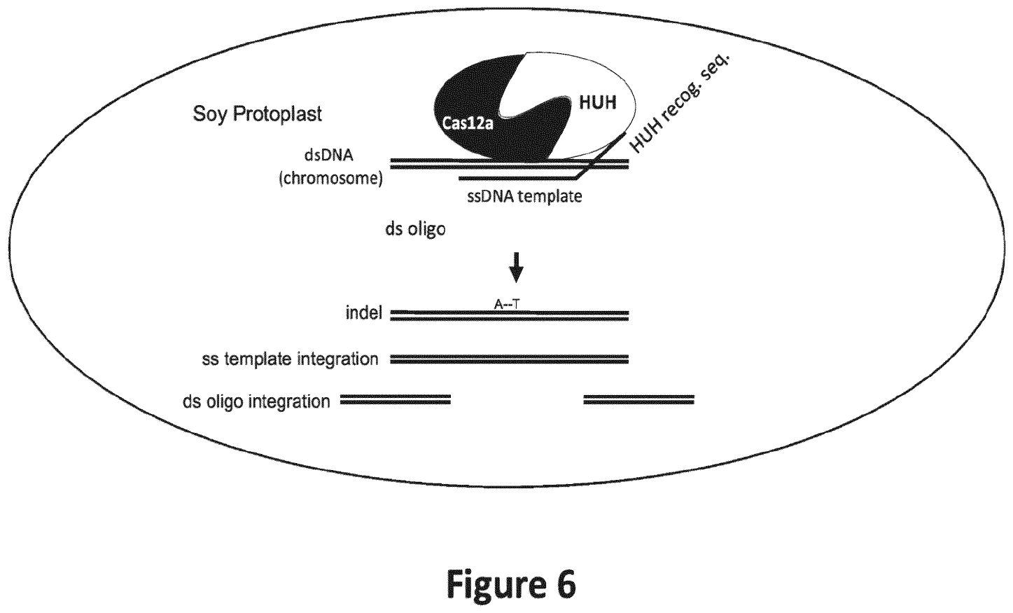

The N- and C-terminal configurations of LbCas12a with FBNYV HUH fusion protein described in Example land FIG. 1B were tested for targeted chromosome cleavage and subsequent DNA repair ex planta, at the GmTS1 target site in soy protoplasts (see FIG. 6). Protoplasts were transformed using various combinations of reagents as listed in Table 7 by standard methods known in the art. The reagents included the “N-terminal” (HUH:cys-free LbCas12a) or the “C-terminal” (cys-free LbCas12a:HUH) protein, the crRNA for the GmTS1 target site, a 70 nt ssDNA template (described in example 4) with or without the 5′ PCV ori extension which facilitates tethering to the HUH domain and a 90 bp dsDNA oligonucleotide (dsDNA oligo).

TABLE 7
Combination of reagents used in the 16 protoplast treatments described in Example 5.

| | Treatments (Tr) | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| HUH:LbCas12a | + | + | − | − | + | + | − | − | − | − | + | − | + | − | − | − |
| LbCas12a:HUH | − | − | + | + | − | − | + | + | − | − | − | + | − | + | − | − |
| crRNA | + | + | + | + | + | + | + | + | + | + | + | + | − | − | + | + |
| GmTS1 | | | | | | | | | | | | | | | | |
| ssTemplate + | + | − | + | − | + | − | + | − | + | − | − | − | − | − | + | − |
| ori | | | | | | | | | | | | | | | | |
| ssTemplate | | + | − | + | − | + | − | + | − | + | − | − | − | − | − | + |
| dsDNA | + | + | + | + | − | − | − | − | − | − | + | + | + | + | + | + |
| Oligo | | | | | | | | | | | | | | | | |

“+” indicates presence.
“−” indicates absence.

After two days of incubation at room temperature, total genomic DNA was isolated. PCR was performed using primers flanking the GmTS1 target site. The amplicons were sequenced using the MiSeq technology (illumina[dot]com). The sequences were analyzed for (1) targeted insertions and deletions (indels), (2) targeted integration of the ssDNA template by either HR or alternative mechanisms, which were collectively labeled as NHEJ-mediated integrations. In this latter case, single-copy and multiple-copy integrations were also distinguished. HR, by definition can integrate only one copy of the template. (3) Multiple-copy and single-copy integrations of the dsDNA oligonucleotide by NHEJ were also quantified.

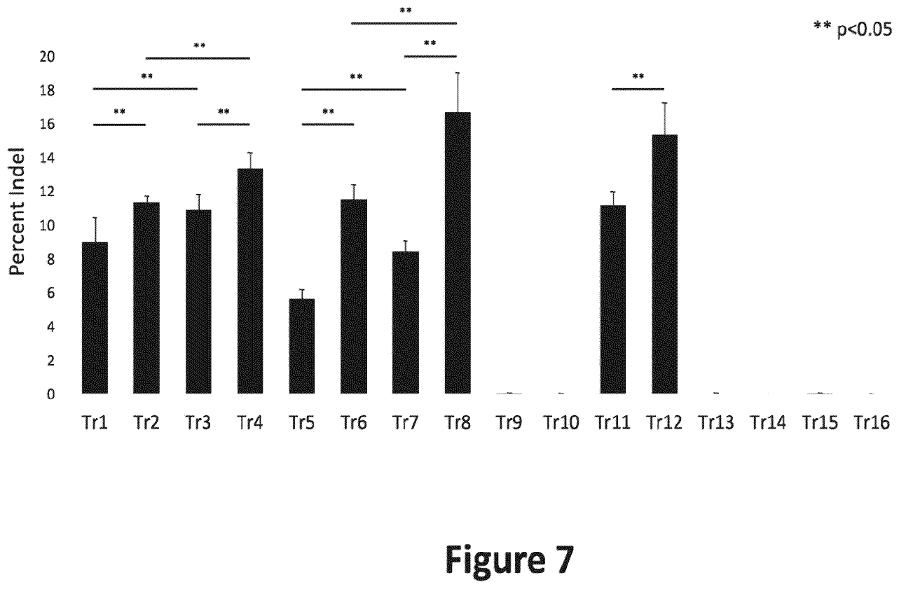

Chromosome cutting was confirmed for both N- and C-terminal enzyme configurations (HUH: cys-free LbCas12a and cys-free LbCas12a: HUH) as evidenced by the presence of indels at the target site in the presence of either fusion protein and cognate gRNA. See for example treatments 1-8, 11 and 12 compared to the control treatments 13-16 in FIG. 7. Cys-free LbCas12a:HUH (“C-terminal” fusion) showed higher indel rates and therefore better cutting rates than HUH:cys-free LbCas12a (“N-terminal fusion”). Tethering the ssDNA template to the HUH domain adversely affected chromosome cutting rates. Without being bound by any particular theory, spatial interference between the DNA template and the Cas12a-chromosome interface could interfere with nuclease activity.

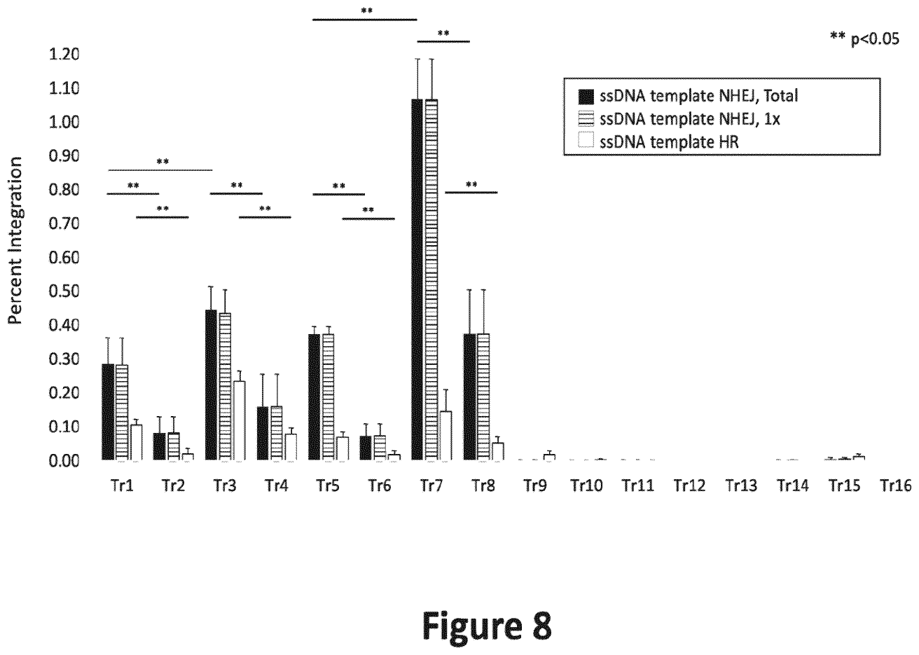

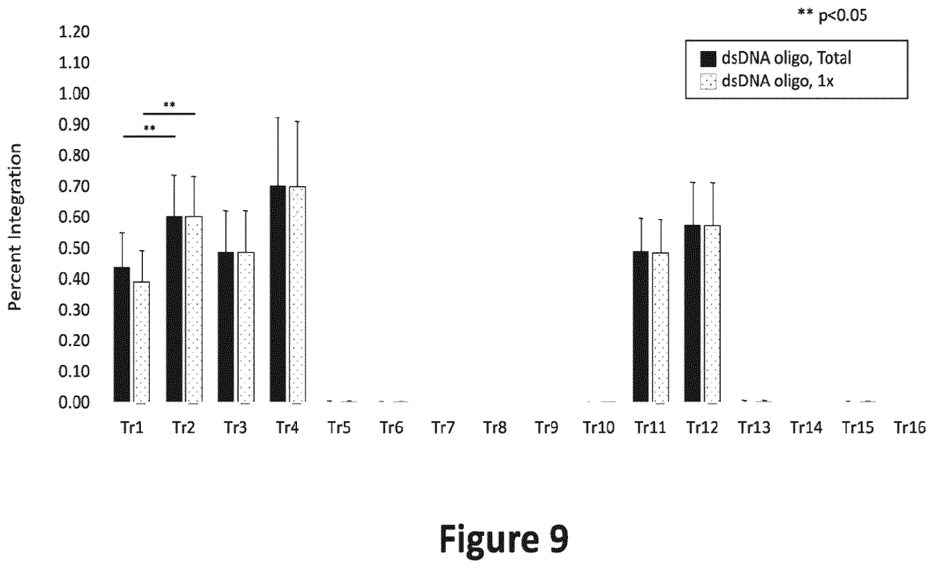

However, template integration by NHEJ was significantly higher when templates were tethered to the HUH domain for both protein configurations (see FIG. 8). Similarly, ssDNA template integration by HR was significantly higher when templates were tethered to the HUH domain for both protein configurations (see FIGS. 8 and Table 8). Integration rates for the dsDNA oligo were higher than those for ssDNA template apart from the effect of HUH tethering (see FIG. 9). In concordance with the indel rates, the C-terminal fusion protein exhibited improved activity compared to the N-terminal fusion protein for integrating the ds oligo, even though this difference was not statistically significant. Also, in line with the findings in the indel rates, targeted integrations of the ds oligo were adversely affected by tethering the ssDNA template to the fusion proteins. In general, NHEJ-mediated integrations of either the ssDNA or dsDNA oligonucleotides showed close correlation with chromosome cutting rates.

TABLE 8
P values for the ssDNA HR dataset with pairwise comparisons among treatments
1 to 16 using random z test (z = δ/δ_SE).

| | Tr 1 | Tr 2 | Tr 3 | Tr 4 | Tr 5 | Tr 6 | Tr 7 | Tr 8 |
|---|---|---|---|---|---|---|---|---|
| Tr 1 | | | | | | | | |
| Tr 2 | 5.9*10^{-05 ‡} | | | | | | | |
| Tr 3 | 1.8*10^{-09 ‡} | 2.4*10⁻⁰¹ | | | | | | |
| Tr 4 | 1.2*10^{-14 ‡} | 3.5*10^{-10 ‡} | 7.0*10^{-06 ‡} | | | | | |
| Tr 5 | 4.8*10^{-07 ‡} | 2.1*10^{-59 ‡} | 1.9*10^{-38 ‡} | 1.8*10^{-35 ‡} | | | | |
| Tr 6 | 9.1*10^{-15 ‡} | 6.0*10⁻⁰¹ | 1.6*10^{-04 ‡} | 3.6*10^{-04 ‡} | 1.1*10^{-67 ‡} | | | |
| Tr 7 | 4.1*10⁻⁰¹ | 5.4*10^{-46‡} | 1.6*10^{-06 ‡} | 5.5*10^{-197 ‡} | 4.2*10^{-09 ‡} | 8.1*10^{-11 ‡} | | |
| Tr 8 | 2.5*10^{-08 ‡} | 7.2*10^{-06 ‡} | 3.7*10^{-05 ‡} | 1.6*10^{-03 ‡} | 1.3*10^{-18 ‡} | 3.5*10^{-05 ‡} | 4.9*10^{-16 ‡} | |
| Tr 9 | 3.3*10^{-34 ‡} | 0.0*10^{-0 ‡} | 5.9*10^{-133 ‡} | 4.7*10^{-172 ‡} | 4.4*10^{-109 ‡} | 5.2*10^{-164 ‡} | 1.0*10^{-150 ‡} | 3.2*10^{-38 ‡} |
| Tr 10 | 7.4*10^{-34 ‡} | 0.0*10^{-0 ‡} | 1.2*10^{-129 ‡} | 1.9*10^{-171 ‡} | 1.9*10^{-113 ‡} | 4.4*10^{-162 ‡} | 3.2*10^{-150 ‡} | 1.4*10^{-38 ‡} |
| Tr 11 | 2.0*10^{-03 ‡} | 6.5*10⁻⁰¹ | 6.1*10⁻⁰¹ | 1.5*10^{-09 ‡} | 1.5*10^{-42 ‡} | 4.6*10⁻⁰¹ | 9.1*10^{-30 ‡} | 4.5*10^{-15 ‡} |
| Tr 12 | 1.4*10^{-06 ‡} | 7.8*10^{-05 ‡} | 1.5*10^{-04 ‡} | 7.7*10⁻⁰² | 3.1*10^{-35 ‡} | 2.1*10^{-04 ‡} | 4.6*10^{-12 ‡} | 2.1*10⁻⁰¹ |
| Tr 13 | 2.1*10^{-34 ‡} | 0.0*10^{-0 ‡} | 2.6*10^{-130 ‡} | 1.2*10^{-190 ‡} | 3.8*10^{-102 ‡} | 5.1*10^{-164 ‡} | 3.8*10^{-175 ‡} | 2.0*10^{-39 ‡} |
| Tr 14 | 8.7*10^{-34 ‡} | 0.0*10^{-0 ‡} | 8.1*10^{-128 ‡} | 4.9*10^{-177 ‡} | 6.9*10^{-110 ‡} | 1.4*10^{-160 ‡} | 1.9*10^{-157 ‡} | 5.4*10^{-39 ‡} |
| Tr 15 | 3.0*10^{-34 ‡} | 0.0*10^{-0 ‡} | 2.2*10^{-131 ‡} | 5.7*10^{-176 ‡} | 1.0*10^{-116 ‡} | 1.2*10^{-166 ‡} | 7.7*10^{-156 ‡} | 5.4*10^{-39 ‡} |
| Tr 16 | 5.8*10^{-34 ‡} | 0.0*10^{-0 ‡} | 3.4*10^{-130 ‡} | 8.4*10^{-174 ‡} | 6.4*10^{-109 ‡} | 1.5*10^{-161 ‡} | 4.4*10^{-153 ‡} | 1.6*10^{-38 ‡} |

| | Tr 9 | Tr 10 | Tr 11 | Tr 12 | Tr 13 | Tr 14 | Tr 15 | Tr 16 |
|---|---|---|---|---|---|---|---|---|
| Tr 1 | | | | | | | | |
| Tr 2 | | | | | | | | |
| Tr 3 | | | | | | | | |
| Tr 4 | | | | | | | | |
| Tr 5 | | | | | | | | |
| Tr 6 | | | | | | | | |
| Tr 7 | | | | | | | | |
| Tr 8 | | | | | | | | |
| Tr 9 | | | | | | | | |
| Tr 10 | 2.0*10⁻⁰¹ | | | | | | | |
| Tr 11 | 4.1*10^{-258 ‡} | 3.6*10^{-261 ‡} | | | | | | |
| Tr 12 | 6.4*10^{-64 ‡} | 2.1*10^{-65 ‡} | 1.0*10^{-06 ‡} | | | | | |
| Tr 13 | 6.2*10⁻⁰¹ | 7.9*10⁻⁰¹ | 8.9*10^{-282 ‡} | 3.5*10^{-64 ‡} | | | | |
| Tr 14 | 3.0*10^{-02 ‡} | 1.4*10⁻⁰¹ | 3.1*10^{-267 ‡} | 1.4*10^{-65 ‡} | 1.1*10⁻⁰¹ | | | |
| Tr 15 | 8.2*10⁻⁰¹ | 7.6*10⁻⁰² | 1.5*10^{-273 ‡} | 9.2*10^{-66 ‡} | 4.5*10⁻⁰¹ | 5.9*10^{-04 ‡} | | |
| Tr 16 | 2.4*10^{-04 ‡} | 1.4*10⁻⁰¹ | 5.8*10^{-260 ‡} | 1.2*10^{-64 ‡} | 2.9*10⁻⁰¹ | 8.9*10⁻⁰¹ | 7.4*10^{-03 ‡} | |

^{‡} indicates P values that are smaller than 0.05 and thus denote significant differences between the corresponding treatments.

### Example 6

### Compatibility of Viral Replication Origins from Various Species With the FBNYV-HUH Endonuclease Fused to cys-free LbCas12a

Both the Faba bean necrotic yellow virus (FBNYV) and the Porcine circovirus

2 (PCV) belong to the class of Arfiviricetes and carry HUH proteins with conserved structures. Likewise, their replication origins (ori) share the same nonamer core sequence (agtattacc) minimally required for recognition and cleavage. See Vega-Rocha et al., 2007, Biochemistry 46:6201 and Timchenko et al., 1999, J. of Virol. 73:10173. The recognition of the FBNYV ori (SEQ IDS NO: 27) and PCV ori (SEQ ID NO: 12) sequences by the Cas 12a fusion proteins carrying the FBNYV HUH endonuclease was investigated. Tral ori sequence (SEQ ID NO: 28) from the unrelated HUH relaxase Tral from the bacterial conjugation F plasmid (see Dostál et al., 2011, Nucleic Acids Res 39:2658) and a “mock” ori sequence comprising a 15bp soy fragment similar in size to the PCV2 ori were incorporated as negative controls. The three ori sequences and the mock ori sequence were fused to the 70-nucleotide long ssDNA template and gel shifts assays similar to those described in Example 2 were performed. Gel-shift assays (see table 6) confirmed that in the presence of cys-free LbCas 12a:HUH, the ssDNA templates comprising the FBNYV ori and PCV ori migrated significantly slower than those comprising the Tral ori or the mock ori. The slower migration is indicative of the formation of cys-free LbCas12a:HUH::ssDNA tethered complexes. Gel shift of tethered oligos indicates that ori sequences from PCV and FBNYV are compatible with the HUH protein of FBNYV, while ori from the distant homolog TraI and a similar-sized mock ori are not.

TABLE 9
Gel shift assay to detect compatibility of the HUH(FBNYV) fusion
proteins with various ori sequences fused to the ssDNA template.

| | | | | Upward |
|---|---|---|---|---|
| | | | | shift in |
| | | | Ori fused | ssDNA |
| | LbCas12a | | to ssDNA | migration |
| Assay type | enzyme | crRNA-TS1 | template | observed |
| Test | Cys-free | + | PCV ori | Yes |
| | LbCas12a:HUH | | | |
| Test | HUH:cys-free | + | PCV ori | Yes |
| | LbCas12a | | | |
| Control | Cys free | + | PCV ori | No |
| | LbCas12a | | | |
| Control | − | − | PCV ori | No |
| Test | Cys-free | + | FBNYV | Yes |
| | LbCas12a:HUH | | ori | |
| Test | HUH:cys-free | + | FBNYV | Yes |
| | LbCas12a | | ori | |
| Control | Cys free | + | FBNYV | No |
| | LbCas12a | | ori | |
| Control | − | − | FBNYV | No |
| | | | ori | |
| Control | Cys-free | + | Tra1 ori | No |
| | LbCas12a:HUH | | | |
| Control | HUH:cys-free | + | Tra1 ori | No |
| | LbCas12a | | | |
| Control | Cys free | + | Tra1 ori | No |
| | LbCas12a | | | |
| Control | — | − | Tra1 ori | No |
| Control | Cys-free | + | Mock ori | No |
| | LbCas12a:HUH | | | |
| Control | HUH:cys-free | + | Mock ori | No |
| | LbCas12a | | | |
| Control | Cys free | + | Mock ori | No |
| | LbCas12a | | | |
| Control | − | − | Mock ori | No |
| Control | Cys-free | + | − | No |
| | LbCas12a:HUH | | | |
| Control | HUH:cys-free | + | − | No |
| | LbCas12a | | | |
| Control | Cys free | + | − | No |
| | LbCas12a | | | |
| Control | − | − | − | No |

“+” indicates presence;
“−” indicates absence.

1. A ribonucleoprotein comprising:
(a) a recombinant polypeptide comprising in the following order:
(i) an amino acid sequence encoding a Cas12a nuclease;
(ii) an amino acid sequence encoding a linker comprising an amino acid sequence at least 93% identical to the amino acid sequence of SEQ ID NO: 3; and
(iii) an amino acid sequence encoding a HUH nuclease selected from the group consisting of a faba bean necrosis yellow virus (FBNYV) HUH endonuclease, a porcine circovirus2 (PCV) HUH endonuclease, a duck circovirus (DCV) HUH endonuclease, a Streptococcus agalactiae replication protein RepB (RepB), a Fructobacillus tropaeoli RepB (RepBm), an Escherichia coli (E. coli) conjugation protein TraI (TraI), an E. coli mobilization protein A (mMobA), and a Staphylococcus aureus nicking enzyme (NES); and
(b) at least one guide nucleic acid.

2. The ribonucleoprotein according to claim 1, wherein the Cas12a nuclease comprises an amino acid sequence having at least 95% sequence identity to an amino acid sequence selected from the group consisting of SEQ ID NO: 1, SEQ ID NO: 15 and SEQ ID NO: 26.

3. The ribonucleoprotein of claim 1, wherein the HUH nuclease comprises an amino acid sequence having at least 95% sequence identity to an amino acid sequence selected from the group consisting of SEQ ID NOs: 2 and 14.

4. The ribonucleoprotein of claim 1, wherein the recombinant polypeptide comprises an amino acid sequence at least 95% identical to an amino acid sequence selected from the group consisting of SEQ ID NOs: 20 and 21.

5. The ribonucleoprotein of claim 1, wherein the ribonucleoprotein further comprises at least one template nucleic acid molecule.

6. The ribonucleoprotein of claim 5, wherein the template nucleic acid molecule comprises single-stranded DN.

7. The ribonucleoprotein of claim 5, wherein the template nucleic acid molecule comprises a nucleic acid sequence encoding an origin of replication (ori) that is capable of hybridizing to the HUH nuclease.

8. The ribonucleoprotein or recombinant nucleic acid of claim 7, wherein the origin of replication comprises SEQ ID NO: 12 or SEQ ID NO: 27.

9. A host cell comprising the ribonucleoprotein of claim 1. cm 10. The host cell of claim 9, wherein the host cell is a plant cell.

11. A method of generating an edit in a target DNA molecule comprising contacting the target DNA molecule with a ribonucleoprotein, wherein the ribonucleoprotein comprises:
(a) a recombinant polypeptide comprising in the following order:
(i) an amino acid sequence encoding a Cas12a nuclease;
(ii) an amino acid sequence encoding a linker comprising an amino acid sequence at least 93% identical to the amino acid sequence of SEQ ID NO: 3; and
(iii) an amino acid sequence encoding a HUH nuclease selected from the group consisting of a faba bean necrosis yellow virus (FBNYV) HUH endonuclease, a porcine circovirus2 (PCV) HUH endonuclease, a duck circovirus (DCV) HUH endonuclease, a Streptococcus agalactiae replication protein RepB (RepB), a Fructobacillus tropaeoli RepB (RepBm), an Escherichia coli conjugation protein TraI (TraI), an E. coli mobilization protein A (mMobA), and a Staphylococcus aureus nicking enzyme (NES);
(b) at least one guide nucleic acid targeted to a selected sequence in the target DNA molecule; and
(c) at least one template nucleic acid molecule that specifically interacts with the HUH nuclease of step (a) (iii),
wherein the ribonucleoprotein generates at least one edit in the target DNA molecule;
wherein covalent tethering of the template nucleic acid molecule to the Cas12a nuclease-HUH nuclease-guide nucleic acid complex promotes genome editing compared to the Cas12a nuclease alone.

12. The method according to claim 11, wherein the Cas12a nuclease comprises an an amino acid sequence having at least 95% sequence identity to an amino acid sequence selected from the group consisting of SEQ ID NO: 1, SEQ ID NO: 15 and SEQ ID NO: 26.

13. A method of generating an edit in a target DNA molecule comprising providing to a cell:
(a) a recombinant polypeptide comprising in the following order:
(i) an amino acid sequence encoding a Cas12a nuclease;
(ii) an amino acid sequence encoding a linker comprising an amino acid sequence at least 93% identical to the amino acid sequence of SEQ ID NO: 3; and
(iii) an amino acid sequence encoding a HUH nuclease selected from the group consisting of a faba bean necrosis yellow virus (FBNYV) HUH endonuclease, a porcine circovirus2 (PCV) HUH endonuclease, a duck circovirus (DCV) HUH endonuclease, a Streptococcus agalactiae replication protein RepB (RepB), a Fructobacillus tropaeoli RepB (RepBm), an Escherichia coli conjugation protein Tral (Tral), an E. coli mobilization protein A (mMobA), and a Staphylococcus aureus nicking enzyme (NES),
or one or more nucleic acid molecules encoding the recombinant polypeptide;
(b) at least one guide nucleic acid targeted to a selected sequence in the targeted DNA molecule of the cell, or at least one nucleic acid molecule encoding the at least one guide nucleic acid; and
(c) at least one template nucleic acid molecule that specifically interacts with the HUH nuclease of step (a) (iii), or at least one nucleic acid molecule encoding the at least one template nucleic acid molecule, wherein the recombinant polypeptide, at least one guide nucleic acid, and at least one template nucleic acid molecule form a ribonucleoprotein, and wherein the ribonucleoprotein generates at least one edit in the target DNA molecule within the cell;
wherein covalent tethering of the template nucleic acid molecule to the Cas12a nuclease-HUH nuclease-guide nucleic acid complex promotes genome editing compared to the Cas 12a nuclease alone.

14. The method according to claim 13, wherein the Cas12a nuclease comprises an an amino acid sequence having at least 95% sequence identity to an amino acid sequence selected from the group consisting of SEQ ID NO: 1, SEQ ID NO: 15 and SEQ ID NO: 26.

15. The method of claim 11, wherein the at least one edit comprises a mutation.

16. The method of claim 15, wherein the mutation comprises a site-directed integration. cm 17. The method of claim 11, wherein the target DNA molecule comprises non-genic DNA.

18. The method of claim 11, wherein the target DNA molecule comprises genic DNA.

19. The method of claim 13, wherein the cell is selected from the group consisting of a corn cell, a soybean cell, a cotton cell, a canola cell, a rice cell, a wheat cell, a sorghum cell, an alfalfa cell, a sugarcane cell, a millet cell, a tomato cell, a potato cell, a cucumber cell, a barley cell, a grass cell, a Setaria cell, an Arabidopsis cell, an Escherichia coli cell, or an algal cell.

20. The method of claim 11, wherein the target DNA molecule is a genomic DNA molecule selected from the group consisting of a nuclear genome DNA molecule, a mitochondrial genome DNA molecule, and a plastid genome DNA molecule.

21. The method of claim 13, wherein the at least one edit comprises a mutation.

22. The method of claim 13 wherein the target DNA molecule comprises non-genic DNA.

23. The method of claim 13, wherein the target DNA molecule comprises genic DNA.

24. The method of claim 13, wherein the target DNA molecule is a genomic DNA molecule selected from the group consisting of a nuclear genome DNA molecule, a mitochondrial genome DNA molecule, and a plastid genome DNA molecule.

25.-72. (canceled)


SEQUENCE LISTING (28 sequences)

SEQ ID NO: 1 - protein, 1227 aa, synthetic construct
  REGION 1..1227: note = Synthetic sequence. Cys-free LbCas12a
SKLEKFTNLYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRIINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDLHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKAPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKIVELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVPLTSAYKELFNKYGINYQQGDIRALLLEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 98 aa, Faba bean necrosis yellow virus
  REGION 1..98: note = FBNYV HUH Endonuclease
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVP

SEQ ID NO: 3 - protein, 16 aa, synthetic construct
  REGION 1..16: note = Synthetic sequence. Linker
SASDTGGSSETGTLDS

SEQ ID NO: 4 - DNA, 3681 bp, synthetic construct
  misc_feature 1..3681: note = Synthetic sequence. Cys-free LbCas12a
  source 1..3681: mol_type = other DNA
agcaaactggaaaaattcaccaacctgtactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgtatcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatctttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgatctgcacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaagcgccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatcgtggaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgagcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgccgctgaccagcgcgtataaagaactgttcaacaaatacggcattaactaccagcagggcgacattcgtgcgctgctgctggaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaaacac

SEQ ID NO: 5 - DNA, 294 bp, synthetic construct
  misc_feature 1..294: note = Synthetic sequence. FBNYV HUH.
  source 1..294: mol_type = other DNA
atggcgcgccaggtgatttgctggtgctttaccctgaacaacccgctgagcccgctgagcctgcatgatagcatgaaatatctggtgtatcagaccgaacagggcgaagcgggcaacattcattttcagggctatattgaaatgaaaaaacgcaccagcctggcgggcatgaaaaaactgattccgggcgcgcattttgaaaaacgccgcggcacccagggcgaagcgcgcgcgtatagcatgaaagaagatacccgcctggaaggcccgtgggaatatggcgaatttgtgccg

SEQ ID NO: 6 - DNA, 48 bp, synthetic construct
  misc_feature 1..48: note = Synthetic sequence. Linker.
  source 1..48: mol_type = other DNA
tctgcttctgatactggtggttcttctgaaactggtactttagattct

SEQ ID NO: 7 - DNA, 4023 bp, synthetic construct
  misc_feature 1..4023: note = Synthetic sequence. Cys-free LbCas12a:linker:FBNYV
  source 1..4023: mol_type = other DNA
agcaaactggaaaaattcaccaacctgtactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgtatcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatctttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgatctgcacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaagcgccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatcgtggaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgagcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgccgctgaccagcgcgtataaagaactgttcaacaaatacggcattaactaccagcagggcgacattcgtgcgctgctgctggaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaaacactctgcttctgatactggtggttcttctgaaactggtactttagattctatggcgcgccaggtgatttgctggtgctttaccctgaacaacccgctgagcccgctgagcctgcatgatagcatgaaatatctggtgtatcagaccgaacagggcgaagcgggcaacattcattttcagggctatattgaaatgaaaaaacgcaccagcctggcgggcatgaaaaaactgattccgggcgcgcattttgaaaaacgccgcggcacccagggcgaagcgcgcgcgtatagcatgaaagaagatacccgcctggaaggcccgtgggaatatggcgaatttgtgccg

SEQ ID NO: 8 - DNA, 30 bp, Solanum tuberosum
  misc_feature 1..30: note = Nuclear localization signal
  source 1..30: mol_type = unassigned DNA
ggtagcaaaaagaggcgtatcaagcaggac

SEQ ID NO: 9 - DNA, 30 bp, Lycopersicon esculentum
  misc_feature 1..30: note = Nuclear localization signal
  source 1..30: mol_type = unassigned DNA
ggatctaagaagcgtaggatcaagcaagat

SEQ ID NO: 10 - DNA, 36 bp, synthetic construct
  misc_feature 1..36: note = Synthetic sequence. HIS tag.
  source 1..36: mol_type = other DNA
atgggcagcagccatcatcaccaccatcaccatatg

SEQ ID NO: 11 - DNA, 28 bp, Escherichia coli
  misc_feature 1..28: note = P.Ec.tac promoter
  source 1..28: mol_type = unassigned DNA
ttgacaattaatcatcggctcgtataat

SEQ ID NO: 12 - DNA, 15 bp, Porcine circovirus
  misc_feature 1..15: note = HUH ori polynucleotide
  source 1..15: mol_type = unassigned DNA
aagtattaccagaaa

SEQ ID NO: 13 - DNA, 348 bp, synthetic construct
  misc_feature 1..348: note = Synthetic sequence. PCV HUH Endonuclease (PCV)
  source 1..348: mol_type = other DNA
agcccgagcaaaaaaaacggccgcagcggcccgcagccgcataaacgctgggtgtttaccctgaacaacccgagcgaagatgaacgcaaaaaaattcgcgatctgccgattagcctgtttgattattttattgtgggcgaagaaggcaacgaagaaggccgcaccccgcatctgcagggctttgcgaactttgtgaaaaaacagacctttaacaaagtgaaatggtatctgggcgcgcgctgccatattgaaaaagcgaaaggcaccgatcagcagaacaaagaatattgcagcaaagaaggcaacctgctgatggaatgcggcgcgccgcgcagccagggccagcgc

SEQ ID NO: 14 - protein, 116 aa, Porcine circovirus
  REGION 1..116: note = PCV HUH
SPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 15 - protein, 1299 aa, unidentified
  REGION 1..1299: note = FnCas12a
  source 1..1299: note = Francisella novicida
SIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 16 - DNA, 3897 bp, synthetic construct
  misc_feature 1..3897: note = Synthetic sequence. FnCas12a
  source 1..3897: mol_type = other DNA
agtatctaccaggagttcgtgaacaaatactccctgagcaaaaccctgcgctttgaactgatcccgcagggtaaaactctggaaaacatcaaagctcgcggcctgattctggacgatgaaaaacgcgcgaaagattacaaaaaagcgaaacagatcatcgataaataccaccagttcttcatcgaagagatcctgtcttctgtgtgcatcagcgaggacctcctgcagaactatagcgatgtctacttcaaactgaaaaagagcgatgacgacaacctccagaaagatttcaaaagcgccaaagacaccattaagaagcagatctccgaatacattaaggattctgagaaatttaaaaacctcttcaaccagaacctgatcgacgcgaaaaaaggccaggagagcgatctgattctgtggctgaaacagtccaaggataacggtattgaactttttaaggctaactctgacatcactgacatcgatgaagccctggaaatcatcaaatcctttaaaggttggaccacttacttcaaaggtttccatgaaaatcgcaaaaacgtttatagctctaacgatattccgacatccatcatctaccgcatcgttgatgacaacttgccgaaattcctggaaaacaaggctaaatatgaatccctgaaagacaaagcgccggaagcaatcaattatgaacagattaaaaaagacctggctgaagaactgaccttcgacatcgactacaaaaccagcgaggttaaccagcgcgtattctctctggacgaagtcttcgagatcgctaacttcaacaactacctgaaccagtccggcatcacgaaattcaacaccattatcggcggcaaatttgtgaacggcgaaaacactaaacgtaaaggcatcaatgagtacatcaatctgtactcccagcagattaatgataaaacactgaagaaatataaaatgagcgtgctgttcaaacagatcctctctgataccgaaagcaagagcttcgttatcgataaacttgaagacgatagcgatgttgttactaccatgcagtccttctatgagcagatcgcagcttttaaaaccgttgaagaaaaatccatcaaagaaaccttatccctgctgttcgatgatctgaaagcacagaaacttgacctgtccaaaatttacttcaaaaacgacaaaagcctgaccgatttaagccagcaggtgttcgatgattatagcgtgattgggacggcagtgctggaatacatcacccaacaaatcgcgccgaaaaacctggacaacccatctaaaaaagagcaggagctgattgcgaaaaagactgaaaaagctaaatatctgtccctggagaccatcaaactggcgctggaggagtttaacaagcatcgcgatattgataagcagtgccgttttgaagaaatcctggcgaacttcgcagcgatcccaatgatttttgacgaaatcgcacagaacaaagataacctggcgcagatcagcatcaaataccagaaccagggtaagaaggacctgctgcaggcatcggccgaagatgatgttaaagcaatcaaagatctcttggatcaaaccaataacctgctgcacaaactgaaaatcttccacatttcccagtctgaagataaggcgaacattctggataaagatgaacacttctatctcgtatttgaggaatgttatttcgagctggcaaacatcgttccgctgtacaacaaaatccgtaactacatcacccagaaaccgtacagcgacgaaaaattcaagctgaatttcgaaaatagcaccttagcgaatggttgggacaaaaataaagaacctgacaacaccgcgatcctgttcatcaaagatgataaatactacctgggcgtaatgaacaaaaagaacaacaaaatctttgacgacaaagcgattaaagaaaacaaaggcgaaggctataaaaagatcgtgtataagctgctgccgggtgcgaacaagatgctgccgaaggtattcttctctgccaaaagcatcaaattctataacccgtcggaagacattctgcgcatccgtaaccactcgacacataccaaaaatggctccccccagaaaggttacgaaaaatttgagttcaacattgaagattgtcgtaaatttatcgacttttacaaacagagcatcagtaaacatccggaatggaaagattttggcttccgcttttcggatacccagcgttacaactccatcgacgagttctaccgcgaagttgaaaaccagggttacaaactgactttcgaaaacatctcggagtcttacatcgattctgttgttaaccagggtaaactgtacctgttccaaatctacaacaaggacttctcagcctacagcaaaggccgcccgaatctgcataccctgtattggaaagcgctcttcgatgaacgtaacctgcaggatgtggtttacaaactgaatggtgaagctgaactcttttatcgtaaacagtcaatcccgaagaaaatcacccacccggcaaaggaagcgattgccaacaaaaacaaggacaaccctaagaaggaaagcgtgttcgaatacgatctgattaaagacaagcgcttcacggaagacaaattctttttccactgcccgatcaccattaactttaaatctagcggggctaacaaattcaacgatgaaatcaacttacttctgaaagaaaaagctaacgacgttcatattctcagtattgaccggggcgaacgtcatctggcatactacaccctggttgacggcaaaggcaacatcattaagcaggacacattcaacatcatcggcaacgatcgtatgaaaactaattaccacgataaactggcggcgatcgagaaagatcgtgatagcgctcgtaaggactggaaaaagatcaacaacattaaagaaatgaaggaaggttacctgagccaggttgtgcatgaaatcgcaaaactggtcatcgaatataacgcgatcgtcgtctttgaagacctgaacttcggttttaaacgtggtcgcttcaaagtcgaaaaacaggtgtaccagaagctggagaaaatgctgatcgaaaaactgaactatctggtattcaaagataacgaatttgataaaaccggtggtgtcctccgtgcttaccaactgaccgctcctttcgaaacctttaagaaaatgggtaagcagaccggcattatctactatgtgccagcaggcttcacctctaaaatttgcccggtcaccggctttgtcaaccaactgtacccaaaatacgaatctgtgagcaaatcccaggaatttttcagcaaattcgataaaatttgttacaacctggataaaggctattttgaatttagctttgactacaaaaactttggcgataaagctgcaaaaggtaaatggaccatcgcgagtttcggttctcgtctgatcaacttccgcaacagcgataaaaaccataactgggacactcgtgaagtttacccgaccaaggaattagaaaaactgctcaaagattacagtatcgaatacggccacggtgaatgtatcaaagcagcaatctgcggtgagtctgataaaaaatttttcgcgaaactgacgtctgtactgaacactattctgcagatgcgtaactccaaaaccggtaccgaactggactacctgatcagcccggttgcggacgttaatggtaactttttcgacagccgtcaggcaccgaaaaacatgcctcaggatgcggacgccaacggcgcgtaccacatcggcctgaaaggcctgatgttgctgggtcgcatcaaaaataaccaggaaggcaaaaaactgaacctggtgattaaaaatgaagaatactttgagttcgtgcagaaccgtaataac

SEQ ID NO: 17 - DNA, 4023 bp, synthetic construct
  misc_feature 1..4023: note = Synthetic sequence. FBNYV:Linker:Cys-free LbCas12a
  source 1..4023: mol_type = other DNA
atggcgcgccaggtgatttgctggtgctttaccctgaacaacccgctgagcccgctgagcctgcatgatagcatgaaatatctggtgtatcagaccgaacagggcgaagcgggcaacattcattttcagggctatattgaaatgaaaaaacgcaccagcctggcgggcatgaaaaaactgattccgggcgcgcattttgaaaaacgccgcggcacccagggcgaagcgcgcgcgtatagcatgaaagaagatacccgcctggaaggcccgtgggaatatggcgaatttgtgccgtctgcttctgatactggtggttcttctgaaactggtactttagattctagcaaactggaaaaattcaccaacctgtactccctgagcaaaaccctgcgcttcaaagcgatcccggttggtaaaacccaggaaaacatcgataacaagcgcctcctggtcgaagacgagaaacgcgcagaggactacaaaggcgtcaaaaagctgctcgatcgctactacctgagcttcatcaacgatgtgttgcacagcatcaaactgaagaacctgaacaactacatcagcctgttccgcaagaaaacccgtaccgaaaaagagaacaaagaactggaaaacctggaaattaacctgcgtaaagaaatcgctaaagcgttcaaaggtaacgagggctacaaatctctgttcaaaaaggacatcatcgaaaccatcctgccggaatttctggatgacaaagatgaaatcgcgctggtgaactcgttcaacggcttcacgaccgcgttcacgggtttcttcgacaaccgcgagaacatgtttagcgaggaagcgaaaagcaccagcatcgccttccgtatcatcaacgaaaacctgacccgctacatcagcaacatggacattttcgagaaggttgacgctatctttgacaaacacgaggttcaggagatcaaggagaaaatcctgaacagcgactacgatgtggaagacttcttcgaaggcgagttcttcaacttcgttctgacccaagagggcatcgacgtttacaacgccatcattggcggcttcgtaaccgaaagcggtgaaaagatcaaagggctgaacgagtatatcaacctgtataaccagaaaaccaaacagaaactgccgaaattcaagccgctgtacaagcaggttctgtccgaccgcgagagcctgagcttctatggcgagggctacacgtccgacgaggaagtgctcgaagtcttccgcaacaccctgaacaagaacagcgagatcttctcgtccatcaaaaagctggagaaactgttcaagaacttcgacgagtactcttctgcgggcatcttcgtgaaaaacggcccggccatcagcacgatttccaaggatatctttggcgagtggaacgtgatccgcgacaaatggaacgctgaatacgacgacatccatctgaagaagaaggcggtcgttaccgaaaaatacgaagatgaccgccgcaagtcttttaaaaagatcggctcgttcagcctggagcagctgcaggaatacgcggacgctgacttgagcgtggtcgagaaactgaaagagatcatcatccagaaggtcgacgaaatctacaaagtgtacggcagtagcgaaaaactgttcgacgctgatttcgtcctggaaaagagcctgaaaaagaacgacgcggtggtggcgatcatgaaggacctgctggacagcgttaagtcgttcgaaaactacattaaagcgtttttcggggaaggcaaagaaaccaaccgcgacgaatctttttacggtgactttgtcctcgcctacgacatcctgctcaaagtcgaccacatctatgacgctatccgcaactacgtgacccagaagccgtacagcaaagacaaattcaagctgtacttccagaacccccagttcatgggcggctgggataaggacaaggaaaccgactaccgcgccaccatcctgcgctacggtagcaaatattacctggcgatcatggacaaaaaatacgccaaatctttgcagaaaatcgacaaggacgacgtgaacggtaactacgaaaaaattaactataaactgctgccgggtccgaacaaaatgctgccgaaagtgttcttcagcaaaaaatggatggcatactacaacccgtctgaagatattcagaaaatctacaaaaacggcaccttcaaaaaaggtgatatgttcaacctgaacgatctgcacaaactgattgatttcttcaaggactcgatctctcgttatccgaaatggtctaacgcgtacgacttcaacttcagcgaaaccgaaaaatacaaagatatcgcgggtttctatcgtgaagttgaagaacagggctacaaagtgtctttcgaatccgcgtccaaaaaggaagtggataaactggtcgaagaaggtaaactgtacatgttccagatctataacaaagacttcagcgataaatcccatggcaccccgaacctgcacaccatgtacttcaaactgctgttcgatgaaaacaaccacggccagatccgtctgtccggcggtgcagaactgtttatgcgccgtgcgtccctgaaaaaagaagagctggtagtacatccggcaaactctccgatcgctaacaaaaacccggacaacccgaagaaaaccaccaccctgagctatgatgtatataaagataaacgtttctccgaagatcagtacgaactgcacatcccgatcgcaattaacaaagcgccgaaaaacatcttcaaaatcaacaccgaagtgcgtgttctgctgaaacacgatgataacccgtacgttattggcatcgaccgtggcgaacgtaacctgctgtacatcgttgtggttgacggtaaaggtaacattgtggaacagtatagcctgaacgaaatcattaacaacttcaacggtatccgtatcaaaaccgattatcacagcctgctggataaaaaagaaaaagaacgttttgaagcgcgtcagaactggaccagcatcgaaaacatcaaagaactgaaagcgggctacatctcgcaggttgttcacaaaatcgtggaactggttgaaaaatacgatgcagttatcgcgctggaagatctgaacagcggtttcaaaaactcacgtgtaaaagttgaaaaacaggtttaccagaaattcgaaaaaatgctgattgataaactgaactatatggtggataaaaaatctaacccgagcgcgactggtggcgcactgaaaggctatcagatcaccaacaagttcgagagcttcaaaagcatgagcacccagaacggtttcatcttctatatcccggcctggctgacctctaaaattgacccgagcactggcttcgtgaacctgctgaaaaccaaatacactagcatcgctgacagcaaaaaattcatctcctcctttgaccgtatcatgtacgtgccggaagaagacctgttcgaatttgcactggattacaaaaacttctcccgcactgacgccgactatattaaaaaatggaaactgtactcttatggtaaccgtatccgtatcttccgtaacccgaagaaaaacaacgttttcgattgggaagaagtgccgctgaccagcgcgtataaagaactgttcaacaaatacggcattaactaccagcagggcgacattcgtgcgctgctgctggaacagtccgataaagcgttctacagctccttcatggcactgatgtccctgatgctgcagatgcgtaacagcattactggccgtaccgatgtggatttcctgatcagcccggttaaaaactctgacggcatcttttacgacagccgtaactacgaagcgcaggaaaacgcgattctgccgaaaaacgcggacgctaacggcgcatacaacatcgcacgtaaagtgctgtgggcgatcggtcagttcaaaaaagcggaagatgaaaaactggataaagtgaaaatcgcgatcagcaacaaagaatggctggaatacgcgcagaccagcgttaaacac

SEQ ID NO: 18 - DNA, 4239 bp, synthetic construct
  misc_feature 1..4239: note = Synthetic sequence. FnCas12a:Linker:FBNYV
  source 1..4239: mol_type = other DNA
agtatctaccaggagttcgtgaacaaatactccctgagcaaaaccctgcgctttgaactgatcccgcagggtaaaactctggaaaacatcaaagctcgcggcctgattctggacgatgaaaaacgcgcgaaagattacaaaaaagcgaaacagatcatcgataaataccaccagttcttcatcgaagagatcctgtcttctgtgtgcatcagcgaggacctcctgcagaactatagcgatgtctacttcaaactgaaaaagagcgatgacgacaacctccagaaagatttcaaaagcgccaaagacaccattaagaagcagatctccgaatacattaaggattctgagaaatttaaaaacctcttcaaccagaacctgatcgacgcgaaaaaaggccaggagagcgatctgattctgtggctgaaacagtccaaggataacggtattgaactttttaaggctaactctgacatcactgacatcgatgaagccctggaaatcatcaaatcctttaaaggttggaccacttacttcaaaggtttccatgaaaatcgcaaaaacgtttatagctctaacgatattccgacatccatcatctaccgcatcgttgatgacaacttgccgaaattcctggaaaacaaggctaaatatgaatccctgaaagacaaagcgccggaagcaatcaattatgaacagattaaaaaagacctggctgaagaactgaccttcgacatcgactacaaaaccagcgaggttaaccagcgcgtattctctctggacgaagtcttcgagatcgctaacttcaacaactacctgaaccagtccggcatcacgaaattcaacaccattatcggcggcaaatttgtgaacggcgaaaacactaaacgtaaaggcatcaatgagtacatcaatctgtactcccagcagattaatgataaaacactgaagaaatataaaatgagcgtgctgttcaaacagatcctctctgataccgaaagcaagagcttcgttatcgataaacttgaagacgatagcgatgttgttactaccatgcagtccttctatgagcagatcgcagcttttaaaaccgttgaagaaaaatccatcaaagaaaccttatccctgctgttcgatgatctgaaagcacagaaacttgacctgtccaaaatttacttcaaaaacgacaaaagcctgaccgatttaagccagcaggtgttcgatgattatagcgtgattgggacggcagtgctggaatacatcacccaacaaatcgcgccgaaaaacctggacaacccatctaaaaaagagcaggagctgattgcgaaaaagactgaaaaagctaaatatctgtccctggagaccatcaaactggcgctggaggagtttaacaagcatcgcgatattgataagcagtgccgttttgaagaaatcctggcgaacttcgcagcgatcccaatgatttttgacgaaatcgcacagaacaaagataacctggcgcagatcagcatcaaataccagaaccagggtaagaaggacctgctgcaggcatcggccgaagatgatgttaaagcaatcaaagatctcttggatcaaaccaataacctgctgcacaaactgaaaatcttccacatttcccagtctgaagataaggcgaacattctggataaagatgaacacttctatctcgtatttgaggaatgttatttcgagctggcaaacatcgttccgctgtacaacaaaatccgtaactacatcacccagaaaccgtacagcgacgaaaaattcaagctgaatttcgaaaatagcaccttagcgaatggttgggacaaaaataaagaacctgacaacaccgcgatcctgttcatcaaagatgataaatactacctgggcgtaatgaacaaaaagaacaacaaaatctttgacgacaaagcgattaaagaaaacaaaggcgaaggctataaaaagatcgtgtataagctgctgccgggtgcgaacaagatgctgccgaaggtattcttctctgccaaaagcatcaaattctataacccgtcggaagacattctgcgcatccgtaaccactcgacacataccaaaaatggctccccccagaaaggttacgaaaaatttgagttcaacattgaagattgtcgtaaatttatcgacttttacaaacagagcatcagtaaacatccggaatggaaagattttggcttccgcttttcggatacccagcgttacaactccatcgacgagttctaccgcgaagttgaaaaccagggttacaaactgactttcgaaaacatctcggagtcttacatcgattctgttgttaaccagggtaaactgtacctgttccaaatctacaacaaggacttctcagcctacagcaaaggccgcccgaatctgcataccctgtattggaaagcgctcttcgatgaacgtaacctgcaggatgtggtttacaaactgaatggtgaagctgaactcttttatcgtaaacagtcaatcccgaagaaaatcacccacccggcaaaggaagcgattgccaacaaaaacaaggacaaccctaagaaggaaagcgtgttcgaatacgatctgattaaagacaagcgcttcacggaagacaaattctttttccactgcccgatcaccattaactttaaatctagcggggctaacaaattcaacgatgaaatcaacttacttctgaaagaaaaagctaacgacgttcatattctcagtattgaccggggcgaacgtcatctggcatactacaccctggttgacggcaaaggcaacatcattaagcaggacacattcaacatcatcggcaacgatcgtatgaaaactaattaccacgataaactggcggcgatcgagaaagatcgtgatagcgctcgtaaggactggaaaaagatcaacaacattaaagaaatgaaggaaggttacctgagccaggttgtgcatgaaatcgcaaaactggtcatcgaatataacgcgatcgtcgtctttgaagacctgaacttcggttttaaacgtggtcgcttcaaagtcgaaaaacaggtgtaccagaagctggagaaaatgctgatcgaaaaactgaactatctggtattcaaagataacgaatttgataaaaccggtggtgtcctccgtgcttaccaactgaccgctcctttcgaaacctttaagaaaatgggtaagcagaccggcattatctactatgtgccagcaggcttcacctctaaaatttgcccggtcaccggctttgtcaaccaactgtacccaaaatacgaatctgtgagcaaatcccaggaatttttcagcaaattcgataaaatttgttacaacctggataaaggctattttgaatttagctttgactacaaaaactttggcgataaagctgcaaaaggtaaatggaccatcgcgagtttcggttctcgtctgatcaacttccgcaacagcgataaaaaccataactgggacactcgtgaagtttacccgaccaaggaattagaaaaactgctcaaagattacagtatcgaatacggccacggtgaatgtatcaaagcagcaatctgcggtgagtctgataaaaaatttttcgcgaaactgacgtctgtactgaacactattctgcagatgcgtaactccaaaaccggtaccgaactggactacctgatcagcccggttgcggacgttaatggtaactttttcgacagccgtcaggcaccgaaaaacatgcctcaggatgcggacgccaacggcgcgtaccacatcggcctgaaaggcctgatgttgctgggtcgcatcaaaaataaccaggaaggcaaaaaactgaacctggtgattaaaaatgaagaatactttgagttcgtgcagaaccgtaataactctgcttctgatactggtggttcttctgaaactggtactttagattctatggcgcgccaggtgatttgctggtgctttaccctgaacaacccgctgagcccgctgagcctgcatgatagcatgaaatatctggtgtatcagaccgaacagggcgaagcgggcaacattcattttcagggctatattgaaatgaaaaaacgcaccagcctggcgggcatgaaaaaactgattccgggcgcgcattttgaaaaacgccgcggcacccagggcgaagcgcgcgcgtatagcatgaaagaagatacccgcctggaaggcccgtgggaatatggcgaatttgtgccg

SEQ ID NO: 19 - DNA, 4242 bp, synthetic construct
  misc_feature 1..4242: note = Synthetic sequence. FBNYV:Linker:FnCas12a
  source 1..4242: mol_type = other DNA
atggcgcgccaggtgatttgctggtgctttaccctgaacaacccgctgagcccgctgagcctgcatgatagcatgaaatatctggtgtatcagaccgaacagggcgaagcgggcaacattcattttcagggctatattgaaatgaaaaaacgcaccagcctggcgggcatgaaaaaactgattccgggcgcgcattttgaaaaacgccgcggcacccagggcgaagcgcgcgcgtatagcatgaaagaagatacccgcctggaaggcccgtgggaatatggcgaatttgtgccgtctgcttctgatactggtggttcttctgaaactggtactttagattctagtatctaccaggagttcgtgaacaaatactccctgagcaaaaccctgcgctttgaactgatcccgcagggtaaaactctggaaaacatcaaagctcgcggcctgattctggacgatgaaaaacgcgcgaaagattacaaaaaagcgaaacagatcatcgataaataccaccagttcttcatcgaagagatcctgtcttctgtgtgcatcagcgaggacctcctgcagaactatagcgatgtctacttcaaactgaaaaagagcgatgacgacaacctccagaaagatttcaaaagcgccaaagacaccattaagaagcagatctccgaatacattaaggattctgagaaatttaaaaacctcttcaaccagaacctgatcgacgcgaaaaaaggccaggagagcgatctgattctgtggctgaaacagtccaaggataacggtattgaactttttaaggctaactctgacatcactgacatcgatgaagccctggaaatcatcaaatcctttaaaggttggaccacttacttcaaaggtttccatgaaaatcgcaaaaacgtttatagctctaacgatattccgacatccatcatctaccgcatcgttgatgacaacttgccgaaattcctggaaaacaaggctaaatatgaatccctgaaagacaaagcgccggaagcaatcaattatgaacagattaaaaaagacctggctgaagaactgaccttcgacatcgactacaaaaccagcgaggttaaccagcgcgtattctctctggacgaagtcttcgagatcgctaacttcaacaactacctgaaccagtccggcatcacgaaattcaacaccattatcggcggcaaatttgtgaacggcgaaaacactaaacgtaaaggcatcaatgagtacatcaatctgtactcccagcagattaatgataaaacactgaagaaatataaaatgagcgtgctgttcaaacagatcctctctgataccgaaagcaagagcttcgttatcgataaacttgaagacgatagcgatgttgttactaccatgcagtccttctatgagcagatcgcagcttttaaaaccgttgaagaaaaatccatcaaagaaaccttatccctgctgttcgatgatctgaaagcacagaaacttgacctgtccaaaatttacttcaaaaacgacaaaagcctgaccgatttaagccagcaggtgttcgatgattatagcgtgattgggacggcagtgctggaatacatcacccaacaaatcgcgccgaaaaacctggacaacccatctaaaaaagagcaggagctgattgcgaaaaagactgaaaaagctaaatatctgtccctggagaccatcaaactggcgctggaggagtttaacaagcatcgcgatattgataagcagtgccgttttgaagaaatcctggcgaacttcgcagcgatcccaatgatttttgacgaaatcgcacagaacaaagataacctggcgcagatcagcatcaaataccagaaccagggtaagaaggacctgctgcaggcatcggccgaagatgatgttaaagcaatcaaagatctcttggatcaaaccaataacctgctgcacaaactgaaaatcttccacatttcccagtctgaagataaggcgaacattctggataaagatgaacacttctatctcgtatttgaggaatgttatttcgagctggcaaacatcgttccgctgtacaacaaaatccgtaactacatcacccagaaaccgtacagcgacgaaaaattcaagctgaatttcgaaaatagcaccttagcgaatggttgggacaaaaataaagaacctgacaacaccgcgatcctgttcatcaaagatgataaatactacctgggcgtaatgaacaaaaagaacaacaaaatctttgacgacaaagcgattaaagaaaacaaaggcgaaggctataaaaagatcgtgtataagctgctgccgggtgcgaacaagatgctgccgaaggtattcttctctgccaaaagcatcaaattctataacccgtcggaagacattctgcgcatccgtaaccactcgacacataccaaaaatggctccccccagaaaggttacgaaaaatttgagttcaacattgaagattgtcgtaaatttatcgacttttacaaacagagcatcagtaaacatccggaatggaaagattttggcttccgcttttcggatacccagcgttacaactccatcgacgagttctaccgcgaagttgaaaaccagggttacaaactgactttcgaaaacatctcggagtcttacatcgattctgttgttaaccagggtaaactgtacctgttccaaatctacaacaaggacttctcagcctacagcaaaggccgcccgaatctgcataccctgtattggaaagcgctcttcgatgaacgtaacctgcaggatgtggtttacaaactgaatggtgaagctgaactcttttatcgtaaacagtcaatcccgaagaaaatcacccacccggcaaaggaagcgattgccaacaaaaacaaggacaaccctaagaaggaaagcgtgttcgaatacgatctgattaaagacaagcgcttcacggaagacaaattctttttccactgcccgatcaccattaactttaaatctagcggggctaacaaattcaacgatgaaatcaacttacttctgaaagaaaaagctaacgacgttcatattctcagtattgaccggggcgaacgtcatctggcatactacaccctggttgacggcaaaggcaacatcattaagcaggacacattcaacatcatcggcaacgatcgtatgaaaactaattaccacgataaactggcggcgatcgagaaagatcgtgatagcgctcgtaaggactggaaaaagatcaacaacattaaagaaatgaaggaaggttacctgagccaggttgtgcatgaaatcgcaaaactggtcatcgaatataacgcgatcgtcgtctttgaagacctgaacttcggttttaaacgtggtcgcttcaaagtcgaaaaacaggtgtaccagaagctggagaaaatgctgatcgaaaaactgaactatctggtattcaaagataacgaatttgataaaaccggtggtgtcctccgtgcttaccaactgaccgctcctttcgaaacctttaagaaaatgggtaagcagaccggcattatctactatgtgccagcaggcttcacctctaaaatttgcccggtcaccggctttgtcaaccaactgtacccaaaatacgaatctgtgagcaaatcccaggaatttttcagcaaattcgataaaatttgttacaacctggataaaggctattttgaatttagctttgactacaaaaactttggcgataaagctgcaaaaggtaaatggaccatcgcgagtttcggttctcgtctgatcaacttccgcaacagcgataaaaaccataactgggacactcgtgaagtttacccgaccaaggaattagaaaaactgctcaaagattacagtatcgaatacggccacggtgaatgtatcaaagcagcaatctgcggtgagtctgataaaaaatttttcgcgaaactgacgtctgtactgaacactattctgcagatgcgtaactccaaaaccggtaccgaactggactacctgatcagcccggttgcggacgttaatggtaactttttcgacagccgtcaggcaccgaaaaacatgcctcaggatgcggacgccaacggcgcgtaccacatcggcctgaaaggcctgatgttgctgggtcgcatcaaaaataaccaggaaggcaaaaaactgaacctggtgattaaaaatgaagaatactttgagttcgtgcagaaccgtaataactga

SEQ ID NO: 20 - protein, 1341 aa, synthetic construct
  REGION 1..1341: note = Synthetic sequence. Cys-free LbCas12a:Linker:FBNYV
SKLEKFTNLYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRIINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDLHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKAPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKIVELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVPLTSAYKELFNKYGINYQQGDIRALLLEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSASDTGGSSETGTLDSMARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVP

SEQ ID NO: 21 - protein, 1341 aa, synthetic construct
  REGION 1..1341: note = Synthetic sequence. FBNYV:Linker:Cys-free LbCas12a
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVPSASDTGGSSETGTLDSSKLEKFTNLYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRIINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDLHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKAPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKIVELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVPLTSAYKELFNKYGINYQQGDIRALLLEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 22 - protein, 1413 aa, synthetic construct
  REGION 1..1413: note = Synthetic sequence. FnCas12a:Linker:FBNYV
SIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNSASDTGGSSETGTLDSMARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVP

SEQ ID NO: 23 - protein, 1413 aa, synthetic construct
  REGION 1..1413: note = Synthetic sequence. FBNYV:Linker:FnCas12a
MARQVICWCFTLNNPLSPLSLHDSMKYLVYQTEQGEAGNIHFQGYIEMKKRTSLAGMKKLIPGAHFEKRRGTQGEARAYSMKEDTRLEGPWEYGEFVPSASDTGGSSETGTLDSSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 24 - DNA, 4293 bp, synthetic construct
  misc_feature 1..4293: note = Synthetic sequence. FnCas12a:Linker:PCV
  source 1..4293: mol_type = other DNA
agtatctaccaggagttcgtgaacaaatactccctgagcaaaaccctgcgctttgaactgatcccgcagggtaaaactctggaaaacatcaaagctcgcggcctgattctggacgatgaaaaacgcgcgaaagattacaaaaaagcgaaacagatcatcgataaataccaccagttcttcatcgaagagatcctgtcttctgtgtgcatcagcgaggacctcctgcagaactatagcgatgtctacttcaaactgaaaaagagcgatgacgacaacctccagaaagatttcaaaagcgccaaagacaccattaagaagcagatctccgaatacattaaggattctgagaaatttaaaaacctcttcaaccagaacctgatcgacgcgaaaaaaggccaggagagcgatctgattctgtggctgaaacagtccaaggataacggtattgaactttttaaggctaactctgacatcactgacatcgatgaagccctggaaatcatcaaatcctttaaaggttggaccacttacttcaaaggtttccatgaaaatcgcaaaaacgtttatagctctaacgatattccgacatccatcatctaccgcatcgttgatgacaacttgccgaaattcctggaaaacaaggctaaatatgaatccctgaaagacaaagcgccggaagcaatcaattatgaacagattaaaaaagacctggctgaagaactgaccttcgacatcgactacaaaaccagcgaggttaaccagcgcgtattctctctggacgaagtcttcgagatcgctaacttcaacaactacctgaaccagtccggcatcacgaaattcaacaccattatcggcggcaaatttgtgaacggcgaaaacactaaacgtaaaggcatcaatgagtacatcaatctgtactcccagcagattaatgataaaacactgaagaaatataaaatgagcgtgctgttcaaacagatcctctctgataccgaaagcaagagcttcgttatcgataaacttgaagacgatagcgatgttgttactaccatgcagtccttctatgagcagatcgcagcttttaaaaccgttgaagaaaaatccatcaaagaaaccttatccctgctgttcgatgatctgaaagcacagaaacttgacctgtccaaaatttacttcaaaaacgacaaaagcctgaccgatttaagccagcaggtgttcgatgattatagcgtgattgggacggcagtgctggaatacatcacccaacaaatcgcgccgaaaaacctggacaacccatctaaaaaagagcaggagctgattgcgaaaaagactgaaaaagctaaatatctgtccctggagaccatcaaactggcgctggaggagtttaacaagcatcgcgatattgataagcagtgccgttttgaagaaatcctggcgaacttcgcagcgatcccaatgatttttgacgaaatcgcacagaacaaagataacctggcgcagatcagcatcaaataccagaaccagggtaagaaggacctgctgcaggcatcggccgaagatgatgttaaagcaatcaaagatctcttggatcaaaccaataacctgctgcacaaactgaaaatcttccacatttcccagtctgaagataaggcgaacattctggataaagatgaacacttctatctcgtatttgaggaatgttatttcgagctggcaaacatcgttccgctgtacaacaaaatccgtaactacatcacccagaaaccgtacagcgacgaaaaattcaagctgaatttcgaaaatagcaccttagcgaatggttgggacaaaaataaagaacctgacaacaccgcgatcctgttcatcaaagatgataaatactacctgggcgtaatgaacaaaaagaacaacaaaatctttgacgacaaagcgattaaagaaaacaaaggcgaaggctataaaaagatcgtgtataagctgctgccgggtgcgaacaagatgctgccgaaggtattcttctctgccaaaagcatcaaattctataacccgtcggaagacattctgcgcatccgtaaccactcgacacataccaaaaatggctccccccagaaaggttacgaaaaatttgagttcaacattgaagattgtcgtaaatttatcgacttttacaaacagagcatcagtaaacatccggaatggaaagattttggcttccgcttttcggatacccagcgttacaactccatcgacgagttctaccgcgaagttgaaaaccagggttacaaactgactttcgaaaacatctcggagtcttacatcgattctgttgttaaccagggtaaactgtacctgttccaaatctacaacaaggacttctcagcctacagcaaaggccgcccgaatctgcataccctgtattggaaagcgctcttcgatgaacgtaacctgcaggatgtggtttacaaactgaatggtgaagctgaactcttttatcgtaaacagtcaatcccgaagaaaatcacccacccggcaaaggaagcgattgccaacaaaaacaaggacaaccctaagaaggaaagcgtgttcgaatacgatctgattaaagacaagcgcttcacggaagacaaattctttttccactgcccgatcaccattaactttaaatctagcggggctaacaaattcaacgatgaaatcaacttacttctgaaagaaaaagctaacgacgttcatattctcagtattgaccggggcgaacgtcatctggcatactacaccctggttgacggcaaaggcaacatcattaagcaggacacattcaacatcatcggcaacgatcgtatgaaaactaattaccacgataaactggcggcgatcgagaaagatcgtgatagcgctcgtaaggactggaaaaagatcaacaacattaaagaaatgaaggaaggttacctgagccaggttgtgcatgaaatcgcaaaactggtcatcgaatataacgcgatcgtcgtctttgaagacctgaacttcggttttaaacgtggtcgcttcaaagtcgaaaaacaggtgtaccagaagctggagaaaatgctgatcgaaaaactgaactatctggtattcaaagataacgaatttgataaaaccggtggtgtcctccgtgcttaccaactgaccgctcctttcgaaacctttaagaaaatgggtaagcagaccggcattatctactatgtgccagcaggcttcacctctaaaatttgcccggtcaccggctttgtcaaccaactgtacccaaaatacgaatctgtgagcaaatcccaggaatttttcagcaaattcgataaaatttgttacaacctggataaaggctattttgaatttagctttgactacaaaaactttggcgataaagctgcaaaaggtaaatggaccatcgcgagtttcggttctcgtctgatcaacttccgcaacagcgataaaaaccataactgggacactcgtgaagtttacccgaccaaggaattagaaaaactgctcaaagattacagtatcgaatacggccacggtgaatgtatcaaagcagcaatctgcggtgagtctgataaaaaatttttcgcgaaactgacgtctgtactgaacactattctgcagatgcgtaactccaaaaccggtaccgaactggactacctgatcagcccggttgcggacgttaatggtaactttttcgacagccgtcaggcaccgaaaaacatgcctcaggatgcggacgccaacggcgcgtaccacatcggcctgaaaggcctgatgttgctgggtcgcatcaaaaataaccaggaaggcaaaaaactgaacctggtgattaaaaatgaagaatactttgagttcgtgcagaaccgtaataactctgcttctgatactggtggttcttctgaaactggtactttagattctagcccgagcaaaaaaaacggccgcagcggcccgcagccgcataaacgctgggtgtttaccctgaacaacccgagcgaagatgaacgcaaaaaaattcgcgatctgccgattagcctgtttgattattttattgtgggcgaagaaggcaacgaagaaggccgcaccccgcatctgcagggctttgcgaactttgtgaaaaaacagacctttaacaaagtgaaatggtatctgggcgcgcgctgccatattgaaaaagcgaaaggcaccgatcagcagaacaaagaatattgcagcaaagaaggcaacctgctgatggaatgcggcgcgccgcgcagccagggccagcgc

SEQ ID NO: 25 - protein, 1431 aa, synthetic construct
  REGION 1..1431: note = Synthetic sequence. FnCas12a:Linker:PCV
SIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNNSASDTGGSSETGTLDSSPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQR

SEQ ID NO: 26 - protein, 1228 aa, unidentified
  REGION 1..1228: note = LbCas12a
  source 1..1228: note = Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 27 - DNA, 12 bp, Faba bean necrotic yellow virus
  misc_feature 1..12: note = FBNYV HUH ori polynucleotide
  source 1..12: mol_type = unassigned DNA
tagtattacccc

SEQ ID NO: 28 - DNA, 22 bp, Escherichia coli
  misc_feature 1..22: note = Tra1 HUH Ori polynucleotide
  source 1..22: mol_type = unassigned DNA
tttgcgtggggtgtggtgcttt